(19) United States
(12) Patent Application Publication
(10) Pub. No.: US 2025/0188298 A1
(43) Pub. Date: Jun. 12, 2025

(54) THREE-DIMENSIONAL CELL-LADEN BIOINK SCAFFOLDS AND METHODS OF MAKING THE SAME UNDER CRYOGENIC CONDITIONS FOR TISSUE ENGINEERING

(71) Applicant: University of Maryland, Baltimore, Baltimore, MD (US)

(72) Inventors: Michal Zalzman, Towson, MD (US); Alok Kumar, Boston, MA (US)

(21) Appl. No.: 18/569,156

(22) PCT Filed: Jun. 13, 2022
(86) PCT No.: PCT/US2022/033220

§ 371 Date: Dec. 11, 2023

Related U.S. Application Data
(60) Provisional application No. 63/209,511, filed on Jun. 11, 2021.

(51) Int. Cl. C09D 11/14; A61L 27/22; A61L 27/24; A61L 27/26; A61L 27/38; B29C 39/00; B29C 39/38; B29C 64/106; B29C 64/30; B29K 105/00; B29K 105/16; B29L 31/00; B33Y 40/00; B33Y 70/10; B33Y 80/00; C09D 11/03; C09D 11/04; C09D 11/106

(52) U.S. Cl. CPC C09D 11/14; A61L 27/222; A61L 27/24; A61L 27/26; A61L 27/3834; B29C 39/003; B29C 39/38; B29C 64/106; B29C 64/30; B33Y 40/00; B33Y 70/10; B33Y 80/00; C09D 11/03; C09D 11/04; C09D 11/106; A61L 2430/02; B29K 2005/00; B29K 2089/00; B29K 2105/0035; B29K 2105/0044; B29K 2105/0088; B29K 2105/16; B29K 2829/04; B29K 2995/0046; B29K 2995/0056; B29K 2995/0077; B29K 2995/0096; B29L 2031/7532

(57) ABSTRACT

The present invention provides three-dimensional, cell-laden bioink scaffolds, methods of making and using the same.

### CROSS-REFERENCE TO RELATED APPLICATIONS

This application is the U.S. National Stage Application under 35 U.S.C. 371 of International Application No.: PCT/US2022/033220, filed on Jun. 13, 2022, which claims the benefit of U.S. Provisional Application No. 63/209,511, filed Jun. 11, 2021.

### STATEMENT OF FEDERALLY SPONSORED RESEARCH AND DEVELOPMENT

This invention was made with government support under Grant Number AR070819 awarded by The National Institutes of Health. The government has certain rights in the invention.

### FIELD OF THE INVENTION

The field of the invention relates to biomaterials and medicine, in particular engraftment of cells and tissue engineering.

### BACKGROUND OF THE INVENTION

Tissue engineering utilizes biological science and materials science & engineering methods to create functional tissue and organ-like devices by combining cells, synthetic or natural biomaterials, and growth factors (Wang et al., The Journal of Bone & Joint Surgery, (2006), 88:1053-1065; Kumar et al., Materials Science & Engineering R: Reports, (2016), 103:1-39). Tissue engineered scaffolds are often used as a shape template and as materials for cell attachment and proliferation (Kumar et al., Polymers for Advanced Technologies, (2019), 30:1189-1197; Nazeer et al., Polymer, (2019), 168:86-94). However, current tissue engineering approaches still lack the ability to provide the conditions to generate thick tissue-like structures. As an important part of the tissue engineering approach, structures are required to create an environment similar to natural tissue. In this context, 3D structures such as organoids and spheroids provide a tunable and tailored environment to the cells. However, these 3D structures are too small (only fraction of a millimeter in size) to mimic the actual tissue properties which hinders their clinical applications. Therefore, methods are still required to create tissue analog large-scale structures.

Additive manufacturing methods have been found useful to create tissue-like structures with architectural complexity (Huh et al., Elsevier, (2020), 1391-1415; Ji et al., Acta Biomaterialia, (2019), 95:214-224; Maiullari et al., Scientific Reports, (2018), 8:13532). As an essential component of these methods, the bioink should mimic the extracellular matrix environment to support the viability, attachment, proliferation, and differentiation of cells (Chimene et al., ACS applied materials & interfaces, (2018), 10:9957-9968; Zheng et al., Advanced healthcare materials, (2018), 7:1701026). Additionally, the physical and mechanical properties of the bioink are important parameters to design scaffolds with high shape fidelity and with no interlayer mixing. Therefore, bioink with high storage modulus is found appropriate in 3D structure fabrication (Gao et al., Biofabrication, (2018), 10:034106). However, in large-scale scaffolds, structural deformation is inevitable due to the weight of successive layers (Ashammakhi et al., Materials Today Bio, (2019), 1:100008; Gillispie et al., Biofabrication, (2020), 12:022003.

The large size of human tissues requires means for rapid fabrication, as slow speed and extended fabrication time can significantly reduce cell viability and stem cell differentiation potential (Sun et al., Biofabrication, (2020), 12:022002). Unfortunately, current methods for faster bioprinting can only be achieved at the expense of the quality of the designed structures (Kumar et al., Journal of biomaterials applications, (2016), 30:1168-1181). A bioink with low viscosity allows a faster printing at the expense of mechanical stability and resolution, while a viscous bioink requires a high extrusion pressure, which imposes high shear stress on the cells during printing, leading to increased cell death (Gao et al., Biofabrication, (2018), 10:034106; Kyle et al., Advanced healthcare materials, (2017), 6:1700264).

Prefabricated tissue-like three dimensional (3D) scaffolds/structures may serve as means to mitigate tissue deterioration and provide a viable solution for tissue repair and their faster recovery. A combined efforts of biology, materials science, and engineering methods are required to create functional tissue and organ-like biomedical devices by combining cells, growth factors, synthetic or natural biomaterials, and external cues (Griffith et al., Science, (2002), 295:1009-14). The 3D structures can be biofabricated either by template casting or by automated fabrication techniques, such as 3D bioprinting. 3D bioprinting allows the use of multiple print heads to create a complex tissue-mimicking structure of various bioinks (biomaterials) and cell types (Kang et al., Nat Biotechnol, (2016), 34:312-9). The ultimate goal of both methods is to create tissue-analogs with structural precision and architectural complexity (Huh et al.,, Elsevier, 1391-1415; Ji et al., Acta Biomaterialia, (2019), 95:214-224; Maiullari et al., Scientific Reports, (2018), 8:13532). Although some advances have been made in the fabrication of 3D structures, it is still not possible to create tissue-mimetic structures accurately using current technology, due to mixing of printed layers, deviation of created structures from the computer aided design (CAD), and slow bioprinting speed (Murphy et al., Nat Biotechnol, (2014), 32:773-85). The successful 3D tissue analogs are limited to only few millimeters in size with minimum structural complexity. Furthermore, crosslinking is needed to improve the strength of the created structure, which involves an exposure of cells-loaded structure to a chemical or radiation for an extended time in non-physiological conditions. This leads to declined cell viability and reduced cellular functions. Therefore, methods are still required to create tissue-analogs with clinically relevant size and structure without causing cell death or anomaly in cellular functions.

Structural deformation, interlayer mixing, and poor shape fidelity are more apparent in larger-sized structures created by conventional bioprinting methods (Ashammakhi et al., Materials Today Bio, (2019), 1:100008; Gillispie et al., Biofabrication, (2020), 12:022003). The structures lager than a few millimeters with high structural fidelity can be achieved by using a bioink of high storage modulus (increases with increase in viscosity in general) (Gao et al., Biofabrication, (2018), 10:034106). However, the high viscosity leads to shear-induced excessive cell death and also decreased printing speed (Gao et al., Biofabrication, (2018), 10:034106).

Therefore, highly organized and accurate tissue structure without interlayer mixing and excessive cell death have yet to be achieved. In this context, a bioink with low viscosity can be used to minimize the shear stress on cells and for the faster printing; however, this comes at the expense of structural stability and resolution (Kyle et al., Advanced healthcare materials, (2017), 6:1700264). The structural stability of the low viscosity bioink can be improved by the crosslinking of each printed layer before the deposition of second layer. However, this slows down the printing process which again leads to reduced cell viability.

Therefore, there is a great need for scientific advances in biofabrication technologies to create cell-loaded multilayered complex 3D structures with high accuracy, viability, and reproducibility.

### SUMMARY OF THE INVENTION

It is to be understood that both the foregoing general description of the invention and the following detailed description are exemplary, and thus do not restrict the scope of the invention.

Described herein is a novel bioink for rapid fabrication of cell-loaded 3D constructs at subzero temperatures. The novel bioink allowed the fabrication of multilayered structures at low extrusion pressure without interlayer mixing and causing cell death. Fabricated at low temperature, frozen structures can be stored in cryogenic conditions for a long time and can be thawed before application. It is shown herein that stem cell-loaded 3D printed structures support the survival, attachment, and differentiation of adult stem cells in the designed scaffolds after prolonged periods of freezing. To demonstrate the feasibility of this novel biofabrication method, a 3D construct using an alginate-collagen-based bioink loaded with mesenchymal stem cells (MSCs) was created. The results herein show that the cells survive the cryogenic printing and long-term storage and can efficiently differentiate into osteoblasts following thawing. The uniquely designed bioink-based structure can provide an off-the-shelf, ready-to-use solution in clinical applications. Furthermore, this method may be tremendously useful to create tissues structures for tissue reconstruction and drug testing.

In one aspect, the invention provides a method of making a frozen, three-dimensional, cell-laden bioink scaffold, comprising:
i) providing an aqueous solution comprising an effective amount of a first biocompatible polymer;
ii) adding an effective amount of a cryoprotectant to the aqueous solution;
iii) optionally adding an effective amount of an agent to the aqueous solution or subjecting the aqueous solution to a condition that promotes crosslinking of the first biocompatible polymer;
iv) adding cells to the aqueous solution to make a cell-laden bioink aqueous solution;
v) casting the cell-laden bioink aqueous solution in a three-dimensional mold at a subzero temperature, bioprinting the cell-laden bioink aqueous solution at a subzero temperature, or infusing the cell-laden bioink aqueous solution into a solid scaffold at a subzero temperature to produce a frozen, three-dimensional, cell-laden bioink scaffold.

In some embodiments, the method further comprises adding to the aqueous solution an effective amount of a second biocompatible polymer, or combining the aqueous solution comprising an effective amount of a first biocompatible polymer with a second aqueous solution comprising an effective amount of a second biocompatible polymer.

In some embodiments, the first biocompatible polymer comprises an alginate.

In some embodiments, the second biocompatible polymer comprises gelatin.

In some embodiments, the second biocompatible polymer comprises collagen.

In some embodiments, the weight ratio of the first biocompatible polymer to the second biocompatible polymer is from about 1:10 to about 10:1.

In some embodiments, the weight ratio of the first biocompatible polymer to the second biocompatible polymer is from about 1:2 to about 2:1.

In some embodiments, the alginate is present in the cell-laden bioink solution in an amount of from about 0.1% by weight to about 3% by weight.

In some embodiments, the alginate is present in the cell-laden bioink solution in an amount of about 0.1% by weight.

In some embodiments, the gelatin is present in the cell-laden bioink solution in an amount of about 0.1% by weight to about 3% by weight.

In some embodiments, the gelatin is present in the cell-laden bioink solution in an amount of about 0.1% by weight.

In some embodiments, the collagen is present in the cell-laden bioink solution in an amount of about 0.15% by weight to about 3% by weight.

In some embodiments, the collagen is present in the cell-laden bioink solution in an amount of about 0.15% by weight.

In some embodiments, the second aqueous solution comprises collagen and is prepared by digesting collagen with pepsin in the second solution.

In some embodiments, the cryoprotectant is selected from DMSO, polyvinylpyrrolidone (PVP), sucrose, glycerol, polyethylene glycol (PEG), ethylene glycol (EG), Ficoll, polyvinyl alcohol, polyglycerol and combinations thereof.

In some embodiments, the cryoprotectant is DMSO and is present in a range of about 2% to about 20% in the cell-laden bioink aqueous solution.

In some embodiments, the cryoprotectant is provided in a carrier solution selected from fetal bovine serum (FBS), human serum, BSA solution (5-10%), and human albumin solution (5-10%).

In some embodiments, the agent that promotes crosslinking is a combination of N-(3-dimethylaminopropyl)-n′-ethylcarbodiimide hydrochloride (EDC) and N-hydroxysuccinimide (NHS), wherein EDC and NHS are added to the aqueous solution and the solution is incubated for a period of time to effect crosslinking.

In some embodiments, the ECD is added to the aqueous solution followed by an incubation step, followed by addition of NHS to the aqueous solution and a further incubation step.

In some embodiments, the aqueous solution is incubated for a period of about 5-60 minutes after EDC is added, and for a period of about 5-60 minutes after NHS is added.

In some embodiments, the second biocompatible polymer is added to the aqueous solution, or the second aqueous solution comprising an effective amount of the second biocompatible polymer is added to the aqueous solution after the first biocompatible polymer has been crosslinked.

In some embodiments, an effective amount of a cryoprotectant is added to the second aqueous solution comprising an effective amount of the second biocompatible polymer before it is combined with the aqueous solution comprising the first biocompatible polymer.

In some embodiments, the three-dimensional mold is a water-soluble polyvinyl alcohol (PVA) mold.

In some embodiments, the subzero temperature is maintained by placing the mold on dry ice or bioprinting on a substrate that is placed on dry ice.

In some embodiments, the cells comprise a heterogeneous population of cells.

In some embodiments, the cells comprise a homogeneous population of cells.

In some embodiments, the cells are selected from genetically engineered cells, differentiated cells, tissue specific stem cells, muscle stem cells, gut stem cells, intestinal stem cells, multipotent stem cells, embryonic stem cells, hematopoietic stem cells, cancer cells, progenitor cells, precursor cells, keratinocytes, melanocytes, neuronal cells, hepatic cells, epithelial cells, cardiomyocytes, cardiac progenitor cells, cardiac stem cells, muscle cells, fibroblasts, osteoblasts, endothelial cells, mesenchymal stem cells, induced pluripotent stem cells, and combinations thereof.

In some embodiments, the cell-laden bioink solution further comprises one or more bioactive molecules.

In some embodiments, the one or more bioactive molecules are capable of being released from the scaffold in a controlled manner.

In some embodiments, the bioactive molecule is selected from a growth factor, cytokine, hormone, drug, immunosuppressant, antibiotic, biologic, antibody, chemotherapeutic agent, and combinations thereof.

In some embodiments, the cell-laden bioink solution further comprises one or more additional components.

In some embodiments, the one or more additional components comprise structures of polymer, or metals, or ceramics, or glass, or composites.

In some embodiments, the one or more additional components comprise extracellular matrix component selected from laminin, collagen, poly D (or L)-lysine, fibronectin, elastin, vitronectins, and combinations thereof.

In some embodiments, the frozen, cell-laden bioink scaffold is capable of being cryopreserved and stored at −80° C. for an extended period of time without significant damage to the scaffold or the cells within the scaffold.

In some embodiments, the method further comprises thawing the frozen, three-dimensional, cell-laden bioink scaffold.

In some embodiments, the frozen, three-dimensional, cell-laden bioink scaffold is thawed at about 37° C. in a culture media.

In some embodiments, the frozen, three-dimensional, cell-laden bioink scaffold is thawed in the presence of an effective amount of a crosslinker. In some embodiments, the crosslinker can be a chemical (such as CaCl₂)), or radiation, or heat.

In some embodiments, the three-dimensional, cell-laden bioink scaffold maintains structural integrity after thawing.

In some embodiments, the three-dimensional, cell-laden bioink scaffold exhibits little or no interlayer mixing during casting or bioprinting and after thawing.

In some embodiments, the thawed, three-dimensional, cell-laden bioink scaffold has a compressive strength of between about 2-3 kPa at 37° C. in submersion conditions.

In some embodiments, the thawed, three-dimensional, cell-laden bioink scaffold has a yield strength of about 1.5-2.0 kPa.

In some embodiments, the thawed, three-dimensional, cell-laden bioink scaffold has a bulk elastic modulus of about 0.05-0.09 kPa.

In some embodiments, the thawed, cell-laden bioink scaffold is capable of supporting cellular proliferation, cellular differentiation, cellular migration, and/or tissue organization.

In another aspect, the invention provides a three-dimensional, cell-laden bioink scaffold produced according to the methods herein.

In another aspect, the invention provides a method of treating a disease or condition in a subject, comprising engrafting the three-dimensional, cell-laden bioink scaffold as described herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Pictorial presentation of scaffold fabricated by casting at subzero temperature and comparison of our novel method scaffold fabrication with the conventional method. We can see the presence of surface irregularities and deformed structure in the case of scaffold prepared by conventional method (top right image). In contrast, fabrication at subzero temperature using our novel bioink leads to a well-defined cylindrical scaffold without any sign of surface anomalies.

FIG. 2. Pictorial presentation of structures printed at subzero temperature and comparison of our novel method of fabrication with the conventional method. The spiral structure created by the conventional method (printing at room temperature) leads to deformation in the structure. While structure created at subzero temperature using our bioink resulted in a well-defined structure with no structural deformation after crosslinking.

FIG. 3 CAD design of a mesh structure (left) and 3D bioprinted mesh structure at subzero temperature at 10 mm/s printing speed (right). The bioprinted structure is similar to the CAD design, which confirms the printability of our novel bioink and efficacy to create complex structures with well-defined porosity.

FIG. 4. Schematic of three layers structure (left) showing the bottom (first), middle (second), and top (third) layers created by hand printing. Results showed the effect of the conventional printing method and printing at subzero temperature using our novel bioink in interlayer mixing and creating structures with distinct features. The structures created by the conventional method (printing at room temperature) lead to extensive interlayer mixing. However, the structure created at subzero temperature using the same bioink resulted in well-defined structure with no interlayer mixing.

FIG. 5. Live/dead assay results of samples after 24 h of storage at −80° C., created using conventional bioink and our novel bioink (with cryoprotectant). Live and dead cells are visible in green and red, respectively. (A, B) The fluorescence microscopy confirmed the extensive cell death in the case of scaffolds created using conventional bioink due to the absence of a cryoprotectant. (C, D) In contrast, due to the presence of cryoprotectant in our novel bioink, we have noted improved function with high cell viability and minimal cell death after printing and storage at subzero temperature.

FIG. 6. Schematic of double layers structure (left) showing the first (red) and second (green) layers, hand-printed using TU167 WT cells prestained in red and green using Green CMFDA and Red CMTPX, respectively. Fluorescence microscopy images show no mixing of cells at the interface of layers. The printing at subzero temperature helps us to create a well-defined cell-loaded multilayered structure without interlayer mixing.

FIG. 7. FT-IR of alginate, collagen, and freeze-dried bioink (crosslinked for 10 min with 0.05 M CaCl₂)). Results confirmed the presence of alginate and collagen in the bioink in its pure form. Also, the triple helix structure of the collagen was intact during the collagen gel preparation and bioink designing, confirmed by comparing the intensity of absorption peaks at 1235 cm⁻¹ and 1450 cm⁻¹.

FIG. 8. Our novel bioink is characterized by low viscosity and yield strength as confirmed by rheology data. (A) The amplitude sweep confirmed the viscoelastic nature of bioink with a higher storage modulus than the loss modulus. (B) Shear stress vs. viscosity curve showed the yielding behavior of viscosity with an increase in the shear stress, visible as a steep decrease in the viscosity at ˜5 Pa. (C) The measurement of viscosity with variation in strain rate confirmed the shear-thinning property of our bioink, which is important for the easy extrusion of bioink through a narrow-gauge needle.

FIG. 9. Stress-strain curve showed an increase in the stress value during the compression test. The results showed an elastic region and yielding before fracture. An increase in the stress after fracture is related to the closure of pores due to compression loading.

FIG. 10. Curve showing the change in swelling ratio (marked with a solid circle) and swelling rate (marked with a solid triangle) of the freeze-dried scaffolds of bioink. An excessive swelling was noted in the first 10 min with an increased swelling rate. After 10 min, an equilibrium was achieved with no further increase in the swelling.

FIG. 11. (A) The dissolution curve showing the decrease in the weight of scaffolds increase with time in long-term dissolution study carried out in 1×PBS (with Ca, Mg) and thus, confirming the degradability of the designed bioink. Results confirmed no abrupt increase in the dissolution. (B) Photographs of scaffolds taken after completion of dissolution study confirmed the integrity of scaffolds with no visible sign of swelling.

FIG. 12. Optical microscopy images of cell-loaded scaffolds after 13 days of culture, kept in culture media (A, B) and osteogenic media (A, B). B and D are the high magnification images of A and D, respectively. Results showed a tissue-like structure with homogenous distribution of cells in bioink matrix. Inset pictures of (A) and (C) are the photographs of scaffolds in culture media and osteogenic media.

FIG. 13. Scanning electron microscope images of samples after 70 days of study. B and D are the high magnification images of A and C, respectively.

FIG. 14. (A) schematic showing the sample and sample holder used in the microCT data collection. B, C, and D are side views (with inset as top view) of the non-treated (no cells) scaffold, scaffold in culture media, and scaffold in osteogenic media. The CaPO₄ crystals (bone mineral) are visible as white in the gray background (scaffold matrix).

FIG. 15. The von Kossa staining of calcium deposited by osteogenically differentiated cells in culture media (A) and osteogenic media (B). As compared to culture media, the higher magnification images showed a higher density of bone mineral in osteogenic media.

FIG. 16. Osteogenic differentiation of MSCs. a. Alizarin red S staining demonstrates extracellular calcium deposits in osteoblasts formed in osteogenic differentiation media (ODM). b. Undifferentiated MSCs grown in complete media (CM) stain negative. c. Real time RT-qPCR analysis validates the differentiation of MSCs in osteogenic media and shows a significant increase in osteoblast markers and d. osteocyte markers compared to control. Data shown as mean±S.E.M. observed in triplicate as demonstrated in at least three donors in independent experiments. Asterisks indicate ** p<0.01, *** p<0.001

FIG. 17. MSCs attach to 3D scaffolds and differentiate into osteoblasts in static conditions. a. Our 3D printed PLA scaffold seeded with MSC derived osteoblasts prior to histochemistry staining. Arrows (yellow) indicate osteoblasts covering the scaffold and filling the pores. Scale bar indicates 30 mm. b. The same scaffold heavily stained with Alizarin red (red), which indicates osteoblast bone cells demonstrating extracellular calcium deposits.

FIG. 18. Representative pictures of samples showing: a. seeding of MSCs using an axial channel scaffold, b. Alizarin red (red) staining indicates bone cells presenting with extracellular calcium deposits. c. the same Alizarin red stained scaffold cut in half to show the distribution of osteoblasts d. A representative microscope image (4×) indicating alizarin red stained cells are filling the pores. These results were reproduced in triplicate in multiple independent experiments.

FIG. 19. Individual Z-stack images, taken at depth intervals of 50 microns from left to right, confirmed the presence of cells and their homogenous distribution in the three-dimensional (3D) scaffold. A. A high number of cells are present and evenly distributed in each layer of the cryogenically stored scaffold, which is similar to the B. control sample, which contains isogenic cells that were not previously frozen and were grown in culture medium.

FIG. 20. A. The 3D printed scaffold loaded with CryoBioInk. B. The 6 cm long and 1.5 cm diameter wide 3D printed bioreactor chamber with a similar 3D printed, CryoBioInk loaded scaffold. The designed scaffold combines PLA components and a polystyrene growth chamber. The bioreactor is designed to allow the secure filling of the fresh media in the chamber and removal of old media using injection or an automated system. The self-healing seal is used to inject and remove culture medium in a closed system and allows the addition of a connector for automatic medium change. The built-in air plug allows the removal of trapped air from the chamber.

FIG. 21. Generation of complex human size 3 D tissues with high accuracy. Tissues and cells can be directly harvested from patients or from donors, processed and stem cells can be isolated and expanded or be used to generate induced pluripotent stem cells (iPSC). At this point, the cells can be either cryopreserved, or taken to generate multilayered 3D structures by mixing into our Cryobioink to be 3D printed, or injected in to scaffolds to generate the desired tissue thickness and shape Then, the tissue-like structures can be either kept for long-term storage until needed, or thawed to allow cells to be differentiated to generate the desired tissue. Finally, the huma size compatible device and be transplanted or taken for in vitro purposes/studies.

FIG. 22. Casting and CryoBioprinting of complex structure with high accuracy. A. Alginate-collagen bioink casted into a water soluble PVA mold at room temperatures. Scaffolds (n=6) were prepared by using conventional bioink methods and were incubated in crosslinking solution for 10 minutes (Top right), while others half (n=6) were casted with Cryobioink and kept at low cryogenic temperatures (−80 C) overnight. Then, frozen scaffolds were thawed by incubation in crosslinking solution at room temperature for 10 minutes. Representative images show surface irregularities and deformed structure in the scaffolds prepared by conventional method (top right). In contrast, fabrication at cryogenic temperature using our novel bioink leads to a well-defined cylindrical scaffold without surface anomalies (bottom right image). B. Human-sized ear created by templet casting of cryobioink in a PVA mold of actual-sized human ear at room temperature (top right) and cryogenic temperature (bottom right). Top scaffolds were crosslinked immediately for 30 min in 0.1M CaCl2), leading as expected to a deformation in the structure. While structure created at cryogenic temperature using our cryobioink resulted in a well-defined structure after crosslinking (30 min in 0.1M CaCl2). C. CAD design of a 2 mm thick structure with diamond-like porosity (left) and 3D bioprinted structures in standard (right; room temperature) and cryogenic conditions using cryobioink (left) at 10 mm/s printing speed. The low panel show a side-by-side representative image of the final print after crosslink D. Representative images of a high-resolution vascular network (top) and a slice of human heart (bottom; showing the right and left ventricular) was 3D printed at cryogenic conditions using cryobioink from a 3D model (CAD) at a speed of 10 mm/s.

FIG. 23. Schematic of double layers structure (left) showing the first (red) and second (green) layers, hand-printed using CryoBioink with cells prestained in red and green using Green CMFDA and Red CMTPX, respectively. Fluorescence microscopy images show no mixing of cells at the interface of layers. The printing at subzero temperature helps us to create a well-defined cell-loaded multilayered structure without interlayer mixing.

FIG. 24. A. Individual Z-stack images, taken at depth intervals of 500 microns from left to right, confirmed the presence of cells and their homogenous distribution in the three-dimensional (3D) scaffold. A. A high number of cells are present and evenly distributed in each layer of the cryogenically stored scaffold, which is similar to the B. control sample, which contains isogenic cells that were not previously frozen and were grown in culture medium. C. Live/dead assay results of samples after 24 h of storage at −80° C., created using conventional bioink and our novel bioink (with cryoprotectant). Live and dead cells are visible in green and red, respectively. The fluorescence microscopy confirmed the extensive cell death in the case of scaffolds created using conventional bioink due to the absence of a cryoprotectant. In contrast, due to the presence of cryoprotectant in our novel bioink, we have noted improved function with high cell viability and minimal cell death after printing and storage at subzero temperature.

FIG. 25. A. FT-IR of alginate, collagen, and freeze dried bioink (crosslinked with 0.05 M CaCl2 for 10 min) Results confirmed the presence of alginate and collagen in the bioink in its pure form. Also, the triple helical structure of the collagen was intact during the collagen gel preparation and bioink synthesis, confirmed by comparing the intensity of absorption peaks at 1235 cm⁻¹ and 1450 cm⁻¹. B. The dissolution curve showing the increase in the weight loss of the scaffolds with time in long-term dissolution study carried out in 1×PBS (with Ca, Mg) and thus, confirming the degradability of the designed bioink. Results confirmed no abrupt increase in the dissolution. (B) Photographs of scaffolds taken after completion of dissolution study confirmed the structural integrity of scaffolds with no visible sign of swelling. C. Cryo-SEM pictures of scaffolds without cells, showing the porous, blood capillaries like structure, can be useful in transporting oxygen and nutrients to the cells growing inside the pores and thus helping tissue like structure formation.

FIG. 26. Representative microscope images (×4) of cell-loaded scaffolds after 13 days of culture, kept in culture media (A, B) and osteogenic media (A, B). B and D are the high magnification images of A and D, respectively. Results showed a tissue-like structure with homogenous distribution of cells in bioink matrix. Inset pictures of (A) and (C) are the photographs of scaffolds in culture media and osteogenic media.

FIG. 27. H&E staining shows the tissue like structure Cytoplasm of cells is visible as pink and nuclei purple The von Kossa staining confirmed the higher deposition of bone like mineral (visible as dark brown color) by osteogenically differentiated cells in osteogenic media as compared to culture media.

FIG. 28. (A) Cubical structure of a 30×30×30 cm by casting in PVA mold in cryogenic conditions followed by crosslinking in culture medium for 30 minutes. (B) Printing at room temperature where 3 layers quickly diffuse at room temperature, and further diffuse during crosslinking, leading to a resultant structure that is deformed, without clear separation of layers. (C) Printing at high cryogenic temperatures (−80 C), where each layer freezes during printing, which allows the layers to be placed side by side without interlayer mixing.

### DETAILED DESCRIPTION OF THE INVENTION

The present invention provides novel methods and materials which can be used to 3D print or cast cells to mimic tissue structure and their microenvironment. Novel compositions are described (“CryoBioInk”) that provide the cells with 3D attachment support and allows freezing at sub-zero temperatures as cells are printed or infused into a mechanically stronger 3D printed porous scaffold. In certain aspects, cells such as stem cells can be incorporated into the CryoBioInk to generate a tissue reconstruction/repair device. Further described herein are compositions comprising mesenchymal stem cells and generated bone-like structures.

The compositions and methods herein have tremendous benefits for individuals with bone pathology, whether from disease, tumor, or trauma. The designed bone grafts provide a viable solution for repair and faster healing of bone defects. 3D printed structures with stem cells-loaded (CryoBioInk) can ensure survival and support cell attachment and differentiation of the adult stem cell-derived bone tissues after prolonged periods of freezing. Importantly, the frozen CryoBioInk tissue replacements can be easily transported without significant damage to the materials or the embedded cells.

The use of the described compositions are not limited to one specific tissue. Multiple cell lines were tested and showed their viability for prolonged periods of time. The CryoBioInk can also act as a substrate for cells to attach, grow, and differentiate to form differentiated tissue and therefore provides an efficient means to generate tissue replacement that will survive in long term storage. Therefore, the present invention enables an off-the shelf tissue replacement solution.

In some embodiments, free-form fabrication methods can be used for the precise placement of layers of cells incorporated in hydrogel and cryopreservation agent and which can be used to generate a tissue structure layer by layer if needed, in freezing conditions. In this method the cell containing BioInk freezes while it is fabricated, therefore, it allows for faster 3D printing compared to existing methods that require a long waiting time for each layer to cure/crosslink. Furthermore, cell viability upon thawing the cells is excellent, as the cells spend far less time while being printed.

The cryogenic conditions during 3D printing allow the generation of very precise structures and layers to form and immediately freeze, preventing the mixing of the layers. The compositions comprising the CryoBioInk provide the cells with 3D attachment support and protect the cells during flash freezing throughout the manufacturing procedure of the tissue. The disclosed CryoBioInk can be 3D printed, casted into a mold or infused into solid scaffolds. The cells in the CryoBioInk can then generate a reconstruction/repair device or be used as a 3D tissue model in precision medicine and drug design.

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

It is contemplated that any method or composition described herein can be implemented with respect to any other method or composition described herein.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

As used herein, the term “about” means plus or minus 10% of the numerical value of the number with which it is being used.

As used herein, a “recipient” is a patient that receives a transplant, such as a transplant containing a scaffold as described herein. The transplanted cells administered to a recipient may be, e.g., autologous, syngeneic, or allogeneic cells.

As used herein, a “donor” is a human or animal from which one or more cells are isolated prior to administration of the cells, or progeny thereof, into a recipient. The one or more cells may be, e.g., a population of cells or stem cells to be engineered, expanded, enriched, or maintained according to the methods of the invention prior to administration of the cells or the progeny thereof into a recipient.

“Expansion” in the context of cells refers to increase in the number of a characteristic cell type, or cell types, from an initial cell population of cells, which may or may not be identical. The initial cells used for expansion may not be the same as the cells generated from expansion.

“Cell population” refers to eukaryotic mammalian, preferably human, cells isolated from biological sources, for example, blood product or tissues and derived from more than one cell.

As used herein, the term “administering,” refers to the placement of a compound, cell, or population of cells as disclosed herein into a subject by a method or route which results in at least partial delivery of the agent at a desired site. Pharmaceutical compositions comprising the compounds or cells disclosed herein can be administered by any appropriate route which results in an effective treatment in the subject.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

As used herein, the terms “subject” and “patient” are used interchangeably and refer to an animal, including mammals such as non-primates (e.g., cows, pigs, horses, cats, dogs, rats etc.) and primates (e.g., monkey and human).

An “effective amount” or “therapeutically effective amount” refers to that amount of a composition described herein which, when administered to a subject (e.g., human), is sufficient to aid in treating a disease. The amount of a composition that constitutes a “therapeutically effective amount” will vary depending on the cell preparations, the condition and its severity, the manner of administration, and the age of the subject to be treated, but can be determined routinely by one of ordinary skill in the art having regard to his own knowledge and to this disclosure. When referring to an individual active ingredient or composition, administered alone, a therapeutically effective dose refers to that ingredient or composition alone. When referring to a combination, a therapeutically effective dose refers to combined amounts of the active ingredients, compositions or both that result in the therapeutic effect, whether administered serially, concurrently or simultaneously.

An “effective amount” is also intended as an amount that is necessary or useful to achieve a certain desired objective or effect. For example, an “effective amount” of a crosslinking agent is an amount that is effective to achieve the desired crosslinking effect.

In some embodiments, the source of the cells used herein are primary cells, and by “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO—S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; and Molt 4 cells.

The term “scaffold” refers to an engineered material platform that in some embodiments can be formed in the shape of tissue that needs to be replaced. The scaffold can be biologically derived or a synthesized material. The scaffold material must be biologically compatible for human implantation. In some embodiments, the scaffold is typically impregnated (seeded) with a patient's cells before implantation. In some embodiments, the scaffold is designed to “degrade” as the cells grow on the scaffold. Typically, in a few days to several months, the scaffold has disappeared and has been replaced by new tissue.

A scaffold is therefore a 3D construct that can serve as temporary support for isolated cells to grow into new tissue either in vitro, i.e., before transplantation back to the host or in vivo, i.e., once implanted.

The design of the scaffold determines the functionality of the construct to a high extent. Although the final requirements depend on the specific purpose of the scaffold, several general characteristics and requirements need to be considered for all designs.

The scaffold should be biocompatible; the scaffold should provoke an appropriate biological response in a specific application and prevent any adverse response of the surrounding tissue. The scaffold materials in some embodiments should degrade in tandem with tissue regeneration and remodeling of the extracellular matrix (ECM) into smaller non-toxic substances without interfering with the function of the surrounding tissue. In some embodiments, the scaffold promotes cell attachment, spreading and proliferation; vital for the regulation of cell growth and differentiation. In some embodiments, the scaffold should have suitable mechanical strength; its strength should be comparable to in vivo tissue at the site of implantation as evidently, a scaffold requires more flexibility or rigidity depending on the application in, e.g., cardiovascular versus bone prostheses. In some embodiments, the scaffold has good transport properties; to ensure sufficient nutrient transport towards the cells and removal of waste products the scaffold should be highly porous with good pore connectivity, however, it should maintain sufficient mechanical strength implying optimization of porosity. The scaffold should ideally be easy to connect to the vascularization system of the host; to ensure good nutrient supply throughout the scaffold post-implantation, the scaffold should be connected to the natural nutrient supplying system suitable surface characteristics; apart from optimal physiochemical properties, research suggests that the introduction of, e.g., surface topography into the scaffold improves tissue organization leading to increased tissue function.

In one embodiment, the invention provides a method of making a frozen, three-dimensional, cell-laden bioink scaffold, comprising:
i) providing an aqueous solution comprising an effective amount of a first biocompatible polymer;
ii) adding an effective amount of a cryoprotectant to the aqueous solution;
iii) optionally adding an effective amount of an agent to the aqueous solution or subjecting the aqueous solution to a condition that promotes crosslinking of the first biocompatible polymer;
iv) adding cells to the aqueous solution to make a cell-laden bioink aqueous solution;
v) casting the cell-laden bioink aqueous solution in a three-dimensional mold at a subzero temperature, bioprinting the cell-laden bioink aqueous solution at a subzero temperature, or infusing the cell-laden bioink aqueous solution into a solid scaffold at a subzero temperature to produce a frozen, three-dimensional, cell-laden bioink scaffold.

Preferably, the solid scaffold has a porous structure.

Methods for 3D printing structures are described in Bhattacharjee et al. Science Advances, e1500655: 1-6 (2015); Bhattacharjee et al. ACS Biomat. Sci. & Eng., 2; 1787-1795 (2016); and O'Bryan et al. Science Advances, 3: e1602800 (2017), which are incorporated by reference for these teachings.

In some embodiments, the method further comprises adding to the aqueous solution an effective amount of a second biocompatible polymer, or combining the aqueous solution comprising an effective amount of a first biocompatible polymer with a second aqueous solution comprising an effective amount of a second biocompatible polymer.

The aqueous solutions comprising an effective amount of a first biocompatible polymer or second biocompatible polymer are not necessarily limiting.

In some embodiments, the first and/or second biocompatible polymer is a hydrophilic or anionic polymer that is capable of swelling in aqueous solution, being crosslinked and forming a hydrogel. In some embodiments, the first or second biocompatible polymer is an alginate. In some embodiments, the first or second biocompatible polymer is selected form collagen, gelatin, elastin, hyaluronate, cellulose, fibrinogen, poly(lactic-co-glycolic acid) (PLGA), poly(glycolic acid) (PGA), poly(lactic acid) (PLA), poly(caprolactone), poly(butylene succinate), poly(trimethylene carbonate), poly(p-dioxanone), and poly(butylene terephthalate), a polyester amide, a polyurethane, poly [(carboxyphenoxy) propane-sebacic acid], poly [bis(hydroxyethyl) terephthalate-ethyl orthophosphorylate/terephthaloyl chloride], a poly(ortho ester), a poly(alkyl cyanoacrylate), poly(ethylene glycol), a microbial polyester, poly((3-hydroxyalkanoate), and a tyrosine derived polycarbonate.

In some embodiments, the first and second biocompatible polymers are dissolved, together or in separate solutions to make aqueous solutions. In some embodiments, the first and/or second biocompatible polymer is dissolved in deionized water. In some embodiments, the first and/or second biocompatible polymer is dissolved in a cell growth medium (e.g., DMEM), that can optionally include serum. In some embodiments, the aqueous solution can include medium with any serum replacement, such as Gibco KnockOut™ Serum Replacement, for example.

The concentrations of the biocompatible polymers in the cell-laden bioink aqueous solution are not necessarily limiting. In some embodiments, the first and/or second biocompatible polymer is present in an amount of from about 0.1% (or less) by weight to about 10% or more by weight. In some embodiments, the first and/or second biocompatible polymer is present in an amount of from about 0.1% (or less) by weight to about 3% by weight. In some embodiments, the first and/or second biocompatible polymer is present in the cell-laden bioink solution in an amount of about 0.1% to about 1.5% by weight by weight. In some embodiments, the first and/or second biocompatible polymer is present in the cell-laden bioink solution in an amount of about 0.1%, 0.15%, 0.2%, 0.3%, 0.4%, 0.5%, 0.6%, 0.7%, 0.8%, 0.9%, 1.0%, 1.1%, 1.2%, 1.3%, 1.4% or 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0% by weight.

In some embodiments, the first biocompatible polymer comprises alginate. The particular alginate that can be used is not limiting.

In some embodiments, the second biocompatible polymer comprises gelatin. The particular gelatin that can be used in not limiting.

In some embodiments, the second biocompatible polymer comprises collagen. The collagen type and source it has been obtained from that can be used is not limiting. In some embodiments, the collagen is digested with an enzyme, such as pepsin. The method of dissolving the collagen not limited. In some embodiments, the second aqueous solution comprises collagen and is prepared by digesting collagen with pepsin in the second solution.

In some embodiments, the cell-laden bioink aqueous solution can include more than two different polymers. In some embodiments, the cell-laden bioink aqueous solution comprises 3, 4, 5, 6, 7, 8, 9 or 10 different polymers.

If more than one polymer is used, the weight ratios of the various polymers is not necessarily limiting. In some embodiments, two polymers are used and the weight ratio of the first biocompatible polymer to the second biocompatible polymer is from about 1:10 to about 10:1. In some embodiments, the weight ratio of the first biocompatible polymer to the second biocompatible polymer is from about 1:2 to about 2:1. In some embodiments, the weight ratio of the first biocompatible polymer to the second biocompatible polymer is from about 1:1.5 to about 1.5:1. In some embodiments, the weight ratio of the first biocompatible polymer to the second biocompatible polymer is about 1:1.

In some embodiments, the alginate is present in the cell-laden bioink solution in an amount of about 0.1% to about 3% by weight. In some embodiments, the alginate is present in the cell-laden bioink solution in an amount of about 0.5% by weight.

In some embodiments, the gelatin is present in the cell-laden bioink solution in an amount of about 0.1% to about 3% by weight. In some embodiments, the gelatin is present in the cell-laden bioink solution in an amount of about 0.5% by weight.

In some embodiments, the collagen is present in the cell-laden bioink solution in an amount of about 0.1% to about 3% by weight. In some embodiments, the collagen is present in the cell-laden bioink solution in an amount of about 0.75% by weight.

In some embodiments, the cryoprotectant is selected from DMSO, polyvinylpyrrolidone (PVP), sucrose, glycerol, polyethylene glycol (PEG), ethylene glycol (EG), Ficoll, polyvinyl alcohol, polyglycerol and combinations thereof. In some embodiments, the cryoprotectant is in a carrier solution that is added to the aqueous solution comprising the biocompatible polymer(s), wherein the carrier solution comprises fetal bovine serum (FBS), cell culture media, human serum, BSA solution (5-10%), and/or human albumin solution (5-10%), or Viaspan®, which is also known as University of Wisconsin solution.

In some embodiments, the cryoprotectant is DMSO and is present in a final concentration in the cell-laden bioink aqueous solution at a range of about 2% to about 20%. In some embodiments, the final concentration of DMSO is about 5%.

In some embodiments, the cell-laden bioink aqueous solution comprises serum, cell culture media or some other complex media, such as Viaspan®, which is also known as University of Wisconsin solution. In some embodiments, the cell-laden bioink aqueous solution comprises serum in a final concentration of from about 1% to about 20%. In some embodiments, the final concentration of serum is from about 5% to about 10%. In some embodiments, the serum concentration can be dependent on the cells. For example, for some cells it is appropriate to use as little as 1% serum while for others, 20% serum is appropriate.

The agent to promote crosslinking of the biocompatible polymers is not limiting. In some embodiments, the agent that promotes crosslinking is a combination of N-(3-dimethylaminopropyl)-n′-ethylcarbodiimide hydrochloride (EDC) and N-hydroxysuccinimide (NHS), wherein EDC and NHS are added to the aqueous solution and the solution is incubated for a period of time to effect crosslinking. In some embodiments, the ECD is added to the aqueous solution followed by an incubation step, followed by addition of NHS to the aqueous solution and a further incubation step. In some embodiments, the aqueous solution is incubated for a period of about 5-60 minutes after EDC is added, and for a period of about 5-60 minutes after NHS is added. In some embodiments, the ECD is added and incubated for about 15 minutes, and the NHS is added and incubated for about 15 minutes.

In some embodiments, the aqueous solution is subjected to a condition that promotes crosslinking, such as chemical, radiation or heat.

In some embodiments, the second biocompatible polymer is added to the aqueous solution, or the second aqueous solution comprising an effective amount of the second biocompatible polymer is added to the aqueous solution comprising the first biocompatible polymer. The second biocompatible polymer or solution comprising the same can be added either before or after the crosslinker has been added. In some embodiments, the crosslinker can be added to the second solution comprising the second biocompatible polymer instead of to the solution comprising the first biocompatible polymer.

The cryoprotectant can be added before or after the crosslinker is added. The cryoprotectant can also be added to the second aqueous solution comprising an effective amount of the second biocompatible polymer before it is combined with the aqueous solution comprising the first biocompatible polymer.

In some embodiments, the cell-laden bioink aqueous solution is cast into a three-dimensional mold. The three-dimensional mold is not limiting. The mold can be water soluble, degradable or permanent. In some embodiments, the mold is a water-soluble polyvinyl alcohol (PVA) mold. In some embodiments, the PVA mold dissolves when it is incubated with a crosslinking solution, or any other aqueous solution. In some embodiments, the mold is made of poly lactic acid (PLA). In some embodiments, the mold is made of titanium. In some embodiments, the mold is made of ceramic. In some embodiments, the mold is made of metal, glass, plastic, or composites.

In some embodiments, the cell-laden bioink aqueous solution is infused into a solid scaffold. In some embodiments, the solid scaffold comprises one or more polymers, such as PLA. In some embodiments, the solid scaffold is prepared by additive manufacturing methods such as 3D printing and 3D bioprinting or my powder metallurgy route such as compression molding with or without sintering.

In some embodiments, the subzero temperature is obtained by placing the mold or solid scaffold, or bioprinting on a substrate, in proximity to a cold substance such as, for example, dry ice or liquid nitrogen. A refrigerated printer can also be used to obtain the subzero temperature. In some embodiments, the subzero conditions enable the shape of the produced scaffold to be stable, and minimizes interlayer mixing, and enables the bioink to have low viscosity which can prevent cell damage and increases their viability.

The cells that can be used are not limiting. The cells can be autologous or allogenic. In some embodiments, the cells comprise a heterogeneous population of cells. In some embodiments, the cells comprise a homogeneous population of cells. In some embodiments, the cells are selected from genetically engineered cells, differentiated cells, tissue specific stem cells, muscle stem cells, gut stem cells, intestinal stem cells, multipotent stem cells, embryonic stem cells, hematopoietic stem cells, cancer cells, progenitor cells, precursor cells, keratinocytes, melanocytes, neuronal cells, hepatic cells, epithelial cells, cardiomyocytes, muscle cells, fibroblasts, osteoblasts, endothelial cells, mesenchymal stem cells, induced pluripotent stem cells, and combinations thereof.

In some embodiments, the cells are primary cells. In some embodiments, the cells are from cell lines.

The cells can be healthy cells, or disease causing cells. In some embodiments, the disease causing cells are cancer cells. In some embodiments, the cancer cells are from a cancer cell line or isolated from a solid tumor.

The type of cancer cells are not limiting, but are preferably cancer cells capable of forming a tumor. As used herein, “cancer” refers to a pathophysiological condition whereby cells are characterized by dysregulated and/or proliferative cellular growth and the ability to induce said growth, which includes but is not limited to, carcinomas and sarcomas, such as, for example, acute lymphoblastic leukemia, acute myeloid leukemia, adrenocortical cancer, AIDS-related cancers, AIDS-related lymphoma, anal cancer, astrocytoma (including, for example, cerebellar and cerebral), basal cell carcinoma, bile duct cancer, bladder cancer, bone cancer, brain stem glioma, brain tumor (including, for example, ependymoma, medulloblastoma, supratentorial primitive neuroectodermal, visual pathway and hypothalamic glioma), cerebral astrocytoma/malignant glioma, breast cancer, bronchial adenomas/carcinoids, Burkitt's lymphoma, carcinoid tumor (including, for example, gastrointestinal), carcinoma of unknown primary site, central nervous system lymphoma, cervical cancer, chronic lymphocytic leukemia, chronic myelogenous leukemia, chronic myeloproliferative disorders, colon cancer, colorectal cancer, cutaneous T-Cell lymphoma, endometrial cancer, ependymoma, esophageal cancer, Ewing's Family of tumors, extrahepatic bile duct cancer, eye cancer (including, for example, intraocular melanoma, retinoblastoma, gallbladder cancer, gastric cancer, gastrointestinal carcinoid tumor, gastrointestinal stromal tumor (GIST), germ cell tumor (including, for example, extracranial, extragonadal, ovarian), gestational trophoblastic tumor, glioma, hairy cell leukemia, head and neck cancer, squamous cell head and neck cancer, hepatocellular cancer, Hodgkin's lymphoma, hypopharyngeal cancer, islet cell carcinoma (including, for example, endocrine pancreas), Kaposi's sarcoma, laryngeal cancer, leukemia, lip cancer, liver cancer, lung cancer (including, for example, non-small cell), lymphoma, macroglobulinemia, malignant fibrous histiocytoma of bone/osteosarcoma, medulloblastoma, melanoma, Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplasia syndromes, myelodysplastic/myeloproliferative diseases, myeloma, nasal cavity and paranasal sinus cancer, nasopharyngeal cancer, neuroblastoma, non-Hodgkin's lymphoma, oral cancer, osteosarcoma, oropharyngeal cancer, ovarian cancer (including, for example, ovarian epithelial cancer, germ cell tumor), ovarian low malignant potential tumor, pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, pregnancy and breast cancer, primary central nervous system lymphoma, prostate cancer, rectal cancer, retinoblastoma, rhabdomyosarcoma, salivary gland cancer, soft tissue sarcoma, uterine sarcoma, Sezary syndrome, skin cancer (including, for example, non-melanoma or melanoma), small intestine cancer, supratentorial primitive neuroectodermal tumors, T-Cell lymphoma, testicular cancer, throat cancer, thymoma, thymoma and thymic carcinoma, thyroid cancer, transitional cell cancer of the renal pelvis and ureter, trophoblastic tumor (including, for example, gestational), unusual cancers of childhood and adulthood, urethral cancer, endometrial uterine cancer, uterine sarcoma, vaginal cancer, viral induced cancers (including, for example, HPV induced cancer), vulvar cancer, Waldenstrom's macroglobulinemia, Wilms' Tumor, and women's cancers.

In some embodiments, the cell-laden bioink solution further comprises one or more bioactive molecules, which are not limiting. In some embodiments, the bioactive molecule is selected from a growth factor, nucleic acids, cytokine, hormone, drug, immunosuppressant, antibiotic, biologic, antibody, chemotherapeutic agent, and combinations thereof. In some embodiments, the bioactive molecule can be a small organic molecule, a nucleic acid, or a polypeptide that can stimulate or promote one or more of cellular invasion, cellular growth, angiogenesis, vascularization, nerve regeneration, or cellular differentiation. The bioactive molecule can be, for example, a growth factor. In one example, the bioactive molecule is one or more growth factors selected from the group consisting of nerve growth factor (NGF), vascular endothelial growth factor (VEGF), platelet derived growth factor (PDGF), neurotrophin-3 (NT-3), brain derived growth factor (BDNF), acidic and basic fibroblast growth factor (FGF), pigment epithelium-derived factor (PEDF), glial derived growth factor (GDNF), angiopoietin, erythropoietin (EPO) and combinations thereof. In another example, the bioactive molecule is a nucleic acid, such as antisense siRNA molecule.

In some embodiments, the one or more bioactive molecules are capable of being released from the scaffold in a delayed or controlled manner. In some embodiments, the bioactive molecule is immediately released from the scaffold.

In some embodiments, the cell-laden bioink solution further comprises one or more additional components. In some embodiments, the one or more additional components comprise polylactic acid (PLA). In some embodiments, the one or more additional components comprise extracellular matrix component selected from laminin, collagen, poly D (or L)-lysine, fibronectin, elastin, vitronectins, and combinations thereof.

In some embodiments, the frozen, cell-laden bioink scaffold is capable of being cryopreserved and stored at −80° C. or colder (such as in liquid nitrogen) for an extended period of time without significant damage to the scaffold or the cells within the scaffold.

In some embodiments, the method further comprises thawing the frozen, three-dimensional, cell-laden bioink scaffold. In some embodiments, the frozen, three-dimensional, cell-laden bioink scaffold is thawed at about 37° C. in a culture media. In some embodiments, the culture media can comprise one or more agents that promote the growth and/or differentiation of the cells in the scaffold.

In some embodiments, the frozen, three-dimensional, cell-laden bioink scaffold is thawed in the presence of an effective amount of a crosslinker. In some embodiments, the crosslinker is CaCl₂).

In some embodiments, the three-dimensional, cell-laden bioink scaffold maintains structural integrity after thawing.

In some embodiments, the three-dimensional, cell-laden bioink scaffold exhibits little or no interlayer mixing during casting or bioprinting and after thawing.

The strength of scaffolds can be increased or decreased by changing the composition of bioink and crosslinking agents concentration and also by adjusting crosslinking time.

In some embodiments, the thawed, three-dimensional, cell-laden bioink scaffold has a compressive strength of between about 2-3 kPa at 37° C. in submersion conditions.

In some embodiments, the thawed, three-dimensional, cell-laden bioink scaffold has a yield strength of about 1.5-2.0 kPa.

In some embodiments, the thawed, three-dimensional, cell-laden bioink scaffold has a bulk elastic modulus of about 0.05-0.09 kPa.

In some embodiments, the thawed, cell-laden bioink scaffold is capable of supporting cellular proliferation, cellular differentiation, cellular migration, and/or tissue organization when cultured in vivo or when engrafted into a subject in vivo.

In some embodiments, the methods can also comprise depositing into the mold or scaffold a vascular structure capable of supporting blood flow. This can be done before, after, or simultaneously with the deposit of the cell-laden bioink scaffold or after thawing it. The vascular structure can have a proximal and distal end in fluid communication. Therefore, in some embodiments, the distal end of the vascular structure is positioned in a location suitable to provide oxygen to the scaffold if blood is pumped into the proximal end of the vascular structure. In some embodiments, the vascular structure comprises an arterial or venous segment from a subject. In other embodiments, the vascular structure is a tissue engineered vessel. For example, the vessel can be produced using autologous or allogenic endothelial cells, progenitor cells, or stem cells.

In another embodiment, the invention provides a three-dimensional, cell-laden bioink scaffold produced according to the methods herein.

In another embodiment, the invention provides a method of treating a disease or condition in a subject, comprising engrafting the three-dimensional, cell-laden bioink scaffold as described herein.

Further disclosed herein are methods to promote wound healing or tissue regeneration in a subject in need thereof, by applying the three-dimensional, cell-laden bioink scaffold as disclosed herein to a wound or tissue of the subject. The three-dimensional, cell-laden bioink scaffold can be applied, for example, to any area of the subject in which tissue regeneration is desired, such as application to an open wound or during the course of a surgical procedure.

In some embodiments, the three-dimensional, cell-laden bioink scaffold are applied to areas of the body with exposed bone or necrotic tissue or diseased/damaged organ.

The tissue or organ is not limiting and can include bone, skin, heart, muscle, liver, kidney, brain, stomach, intestine, pancreas, ovary, etc.

In some embodiments, the three-dimensional, cell-laden bioink scaffold is applied to bone tissue, and can include, for example, mesenchymal stem cells, osteoblasts, precursor cells, and/or iPS cells. In some embodiments, where the tissue is bone, the cells can comprise bone marrow cells, or marrow-like cells. In some embodiments, the cells comprise mesenchymal stem cells (MSCs), hematopoietic stem cells (HSCs), osteoblasts, osteocytes, angioblasts, or any combination thereof. In some embodiments, the scaffold further comprises growth factors that can promote angiogenesis, osteogenesis, and/or chondrogenesis, such as a vascular endothelial growth factor (VEGF), a fibroblast growth factor (FGF), an epidermal growth factor (EGF), an insulin-like growth factor (IGF), a bone morphogenetic protein 2 (BMP), or any combination thereof.

In some embodiments, the three-dimensional, cell-laden bioink scaffold is applied to cardiac tissue. In some embodiments, the subject has suffered a myocardial infarction or complications associated with myocardial infarction, or has or is at risk of developing heart failure or arrythmias. In some embodiments, the scaffold is seeded with cardiomyocytes, stem cells, precursor cells, and/or iPS cells. In some embodiments, the scaffold comprises one or more extracellular matrix components. See, e.g., Kato et al., Journal of Materials Science: Materials in Medicine (2021) 32:54; Kupfer et al., Circulation Research. 2020; 127:207-224; Alonzo et al., Translational Research, 2019; 211:64-83, which are incorporated by reference herein. In some embodiments, the compositions can be used in a patient with curretage or for transmural infarct treatment, preferably in the treatment of myocardial infraction, and more preferably wherein the treatment comprises cardiac tissue regeneration. In some embodiments, the invention relates to a method for treating a patient with myocardial infraction which comprises the implantation of a cardiac patch, that is made from a three-dimensional, cell-laden bioink scaffold as defined above, in the heart pericardium, preferably in the surface of the heart pericardium by suture of the support or through the heart pericardium and myocardium by suture of the support.

The three-dimensional, cell-laden bioink scaffolds disclosed herein can be removed or remain in place. The biocompatible polymer can be biodegradable and in such cases will gradually dissolve, leaving behind a new network of cells and vasculature formed from the cells.

In another embodiment, the invention provides a method of screening for effective therapeutic agents, comprising contacting the three-dimensional, cell-laden bioink scaffold as described herein with an amount of an agent to be tested, and determining whether there is a therapeutic effect on the cells of the three-dimensional, cell-laden bioink scaffold. The screening assay can be done in vivo, in vitro, or ex vivo.

In some embodiments, the screening methods are performed on three-dimensional, cell-laden bioink scaffolds that comprise cancer cells and the therapeutic agents tested are potential anti-cancer therapeutics. In some embodiments, the cells of the three-dimensional, cell-laden bioink scaffold are assayed for cell cycle arrest, apoptosis, differentiation, or cell death.

All publications herein are incorporated by reference to the same extent as if each individual publication or patent application was specifically and individually indicated to be incorporated by reference.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLES

### Example 1. A Novel Cell-Laden Bioink for Rapid Cryogenic 3D-Biofabrication and Storage of Living Tissues

3D-printing is a promising tool for regenerative medicine to address the urgent demand of tissues and organs suitable for transplantation. Although the conditions to generate 3D-structures such as organoid and spheroids, have been established for many cell types, these structures are too small (only fraction of a millimeter in size) to have applications in tissue repair or organ replacement. Advanced biofabrication techniques, such as 3D bioprinting, can be used in the design of clinically-relevant size scaffolds. However, poor shape fidelity, extensive interlayer mixing of cells, and slow printing, prevent creating complex-shaped large tissues using currently available bioprinting techniques. Furthermore, current crosslinking methods and the long time the cells spend in non-physiological conditions during printing, hinder cell viability and lead to massive cell death. To address these issues, we have developed a novel and rapid procedure to generate physiologically-relevant sized cells-loaded 3D structures by casting or by 3D bioprinting at subzero temperatures. Our results show that the tissue replacements created from our designed bioink can then be cryopreserved and stored (or transported) at −80° C. for an extended period of time. The cells can then be revived, after prolonged storage in cryogenic conditions, without significant damage to the scaffolds or the embedded cells within the scaffold. Our approach paves the way for generating off-the-shelf tissue-engineered medical devices for regenerative medicine and may have tremendous benefits for individuals with tissue pathology, whether from disease, tumor, or trauma.

### Materials and Methods

Sodium alginate (cat. no. ICN218295, MP Biomedicals), calcium chloride anhydrous (cat. no. C1016, Sigma Aldrich), gelatin type B (cat. no. G9391, Sigma Aldrich), Collagen (cat. no. C9879, Sigma Aldrich), pepsin (cat. no. P7012, Sigma Aldrich), N-(3-dimethylaminopropyl)-n′-ethylcarbodiimide Hydrochloride (EDC, cat. no. 03449, Sigma Aldrich), N-hydroxysuccinimide (NHS, cat. no. 130672, Sigma Aldrich), Dimethyl sulfoxide (DMSO, cat. no. D2650, Sigma Aldrich), 1×PBS (with Ca, Mg, cat. no. 14040133, Gibco), fetal bovine serum (FBS, cat. no. 631106, Takara Bio), and high glucose Dulbecco's modified Eagle's medium (DMEM, cat. no. 11965118, Gibco) were used. Ultimaker S5 3D Printer was used to design water-soluble polyvinyl alcohol (PVA) molds. The PAV (cat. no. UMFPV0000) filament of 2.85 diameter was procured from the Ultimaker.

### Synthesis of Novel Bioink for the Fabrication at Subzero Temperature

We have designed two novel bioinks, 50 (1 wt. % alginate)-50 (1 wt. % gelatin) (50Alg-50Gel) and 50 (1 wt. % alginate)-50 (1.5 wt. % collagen) (50Alg-50Col), supplemented with freezing media (containing 10% DMSO). For the bioink, 2 wt. % alginate was prepared by dissolving 0.2 g of alginate in 10 ml filtered deionized (DI) water at room temperature overnight at slow stirring condition. The 2 wt. % gelatin was prepared by dissolving 0.2 g of gelatin in 10 ml DI water at 37° C. temperature. The 3 wt. % collagen was prepared in DI water by digesting collagen using pepsin in acidic conditions at 37° C. For this, first 10 mg pepsin was dissolved in 10 ml DI water (pH 2, adjusted using conc. HCl) at 37° C., followed by dissolving 300 mg collagen in the solution of pepsin at 37° C. for 3 h. After preparation, the pH of the collagen gel was adjusted to 7.4 using 10M NaOH. The solution of alginate, gelatin, and collagen was stored at 6° C.

Next, 2 wt. % alginate, 2 wt. % gelatin, and 3 wt. % collagen was mixed in the equal amount of freezing media (10% DMSO in FBS (v/v)) for 10 min, to make 1 wt. % alginate, 1 wt. % gelatin, and 1.5 wt. % collagen, respectively. To prepare 10 ml of 50Alg-50Gel bioink, 12.5 mg EDC was mixed in 5 ml 1 wt. % alginate for 15 min at room temperature and slow stirring condition, followed by adding 7.5 mg NHS and mixing for another 15 min. Thereafter, 1 wt. % gelatin was added and mixed for 2 min to make 50Alg-50Gel bioink. Moreover, to prepare 10 ml of 50Alg-50Col bioink, 12.5 mg EDC was mixed in 5 ml 1 wt. % alginate for 15 min at room temperature and slow stirring condition, followed by adding 7.5 mg NHS and mixing for another 15 min. Then, 1.5 wt. % collagen was added and mixed for 2 min to make 50Alg-50Col bioink. The cells (suspended in 100 μl freezing media) were added in the last step of bioink preparation to make a cell-loaded bioink. The excess 100 μl was adjusted by mixing 2 wt. % gelatin (or 3 wt. % collagen) in less amount of freeing media when preparation 1 wt. % gelatin (or 1.5 wt. % collagen).

The designed bioinks were kept at room temperature, and scaffolds were created by casting in a PVA mold or bioprinting at subzero temperature. The designed scaffolds were stored at −80° C. until use.

### Fabrication of Three-Dimensional Scaffolds

Three-dimensional (3D) structures of 50Alg-50Gel and 50Alg-50Col were fabricated either by casting of bioink in water-soluble polyvinyl alcohol (PVA) mold or by 3D bioprinting at subzero temperature. In this study, dry ice (−78.5° C.) was used to maintain the subzero temperature. A positive displacement-based syringe extruder with ½″ blunt stainless-steel needle of 0.5 mm inner diameter was used for the dispensing of cell-loaded bioink in PVA mold kept on dry ice. To optimize biofabrication parameters, 50Alg-50Gel was extruded manually in a well plate kept on dry ice to create multilayered layers structures without cells. Bioprinting in this condition allows flash freezing of printed structures. The validate the efficacy of our novel bioink in creating scaffolds using an automated fabrication system and printability, 3D mesh structure 50Alg-50Gel was created at a printing speed of 10 mm/s and at subzero temperature. The designed structures were stored at −80° C. After 24 h, frozen scaffolds were directly thawed at 37° C. in a culture media supplemented with a crosslinker (0.05 M CaCl₂)). For the comparison, similar structures were created at ˜37° C., followed by crosslinking for 10 min in 0.05 M CaCl₂).

Storage of 3D Scaffolds at −80° C. and Recovery of Scaffolds with Cells after Thawing

The created frozen scaffolds of our bioink (with DMSO) were stored at −80° C. To see the effect of DMSO in protecting the cells during freezing, a 50Alg-50Gel without DMSO was also prepared and created scaffolds were stored at −80° C. To study the cell viability, after 24 h, frozen scaffolds were thawed for 10 min at 37° C. in a culture media containing 0.05 M CaCl₂) as a crosslinker. After crosslinking, samples were washed twice with culture media and then incubated in a fresh culture media in a CO₂ incubator for 2 h to dissolve the PVA mold completely. No change in the pH of culture media was noted due to the dissolution and presence of PVA. Culture media was replaced with fresh culture media every 3.5 days thereafter.

### Cell Viability Assay

Samples were thawed and incubated in culture medium for 2 days. Then wholemount staining of the 3D structures was performed using live/dead kit (LIVE/DEAD™ cell imaging kit, Cat. No. R37601, Invitrogen) according to the manufacturer protocol in order to visualize the live (green) and dead cells (red).

Investigation of the Microstructure of 3D Scaffolds and the Phases Present within it

Fourier transform-infrared spectroscopy (FT-IR) was used to know the functional groups and, therefore, to determine the composition of the designed bioink. The dried and grounded bioink samples were used to record the FT-IR data from 4000 to 600 cm⁻¹. The obtained data were compared with as purchased alginate and collagen.

To determine the microstructure and pore morphology of the designed bioink as well as cells within the scaffolds, the scaffolds (without and without cells) were fixed for 16 h in 10% formaldehyde at 4° C. samples. Thereafter, a thin section of the scaffolds was adhered to a metallic stub using cryoembedding solution, followed by flash freezing in liquid nitrogen. Prior to observation, the surface of frozen samples was fractured and sublimated to expose the inner microstructure. Thereafter, samples were gold coated (required to minimize the image distortion caused by the charging effect) and observed under a scanning electron microscope (SEM) in secondary electron mode, operated at an accelerating voltage of 5 kV and a working distance of 6 mm. A low temperature of −130° C. was maintained inside the SEM throughout the imaging process.

### Rheological Properties of Designed Bioink

The determination of rheological properties helps us to tailor the physical properties of the bioink, required for the rapid bioprinting of 3D structures without causing the death of cells. A dynamic stress rheometer with a cone and plate geometry (Cone angle: 1 degree and truncation gap: 26 μM, model: HR30, TA Discovery, USA) was used to measure storage and loss modulus at room temperature. A bioink volume of 300 μL was used in this study. First, amplitude sweep (Oscillation strain sweep: 10⁻² to 10²) at a fixed value of frequency 1 Hz was carried out to know the value of storage modulus and loss modulus. The data was used to know the viscoelastic properties of bioink and recovery of bioink after the transition from a more solid-like phase to a more liquid-like phase during extrusion. Furthermore, shear rate vs. shear stress and shear stress vs. viscosity were measured to determine the properties of bioink under the effect of stress.

### 1.6. Shape Fidelity and Interlayer Mixing During 3D Scaffold Fabrication

To study the shape fidelity and interlayer mixing, multilayered structures were created by extruding bioink at subzero temperature, followed by storing the printed structures at −80° C. for 24 h. Next day, frozen structures were thawed in 0.05 M CaCl₂) at room temperature.

To further investigate the intermixing, TU167 WT cells were prestained in red (Red CMTPX, Cat. No. C34552, Invitrogen) and green (Green CMFDA, Cat. No. C7025, Invitrogen). Briefly, pre-stained cells were added in the bioink and printed manually to create multilayered structures at subzero temperature. After 24 h of storage at −80° C., these frozen structures were thawed in culture media containing 0.05 M CaCl₂). On the 2^{nd} day of incubation in culture media, structures were observed under the fluorescence microscope to know the interlayer mixing.

### Mechanical Properties

In order to measure the compressive strength of the scaffolds of bioink, cylindrical shaped scaffolds of dimension 9 mm×6 mm (diameter×length). At least five samples were tested to obtain a statistically relevant data. Based on ISO 604:2002 inequality criterion for maximum nominal compressive strain was used to determine the scaffolds dimension.

$${ε}_{c} \leq 0 \cdot 4 \frac{{diameter}^{2}}{{length}^{2}}$$

Note that the theoretical strain values lie between 0 to 1. Or in percentage 0 to 100%. Scaffolds were fabricated by casting in PVA mold, followed by storage if the scaffolds at −80° C. The next day, scaffolds were thawed in 0.05M CaCl₂), followed by washing twice with 1×PBS (with Ca, Mg). Furthermore, 1×PBS was changed after 30 min, 2 h and 24 to ensure complete removal of PVA mold. The next day, these scaffolds were used to measure the compressive strength and to estimate the elastic modulus. A dynamic mechanical analyzer (DMA, model: DMA850, TA Discovery) was used to measure the mechanical properties and testing was carried out at 37° C. in submersion condition in 1×PBS (with Ca, Mg). Testing was performed at rate control-strain ramp mode at a strain rate of 1%/min and 0 kN preload.

### Scaffold Stability During the Incubation of 3D Scaffolds in Aqueous Media

To determine the stability and integrity of the designed scaffolds, dissolution study in 1×PBS (with Ca, Mg) was conducted for 7, 14, 21, and 28 days. The crosslinked cylindrical-shaped scaffolds (of dimension 9 mm×6 mm) were washed with 1×PBS to ensure the removal of excess CaCl₂) and PVA mold. At least three samples were used in each category. After taking initial weight, scaffolds were kept in a CO₂ incubator during the dissolution study. After 7, 14, 21, and 28 days of incubation, the solution from each well was carefully removed, and the final weight was measured.

Furthermore, swelling behavior of scaffolds after thawing was studied in 1×PBS (with Ca, Mg) at 37° C. For this, crosslinked samples were freeze-dried, and weight was measured (Wd). Afterward, samples were immersed in culture media for 30 min. Swollen samples were removed from the culture media, followed by careful removal of surface liquid using tissue paper before measuring the weight (Wt). The process was repeated every 5 min for the first 30 min and then every 30 min until a swelling equilibrium was reached. Degree of swelling (swelling ratio), swelling rate, equilibrium swelling ratio, and percentage equilibrium liquid content were measured using the following equations:

$$Swelling ratio = \frac{{W}_{t} - {W}_{d}}{{W}_{d}} = degree of swelling \qquad ( 1 )$$

$$Swelling rate = weight change per unit time = \frac{{W}_{t + Δ t} - {W}_{t}}{Δ t} \qquad ( 2 )$$

$$Equilibrium swelling ratio = \frac{{W}_{equ} - {W}_{d}}{{W}_{d}} \qquad ( 3 )$$

$$Percentage equilibrium water content = \frac{{W}_{equ} - {W}_{d}}{{W}_{equ}} \times 100 \qquad ( 4 )$$

Where, Wₜ, W_{d}, W_{t+Δt}, and W_{equ} are the weight of swollen samples at time t, the weight of dried samples, weight of the swollen samples at time interval Δt, and weight of the swollen samples at equilibrium state, respectively

### Osteogenic Differentiation of MSCs Grown Inside the 3D Scaffolds

To demonstrate the efficacy of the bioink to not only support cell viability during and after printing, but also supporting stem-cell differentiation, we assessed our MSC bone differentiation in a long-term in vitro study. Alginate-Gelatin or a Alginate-Collagen bioinks were loaded with mesenchymal stem cells (MSCs) and casted in a cylindrical PVA mold with (10 mm diameter×5 mm thick) at density of 10⁶ cells/per sample. The scaffolds were then stored at −80° C. for at least 24 hours. Thawing and crosslinking was performed by incubation for 10 minutes in a culture media containing 0.05 M CaCl₂) at 37° C. Next, crosslinking media was changed with culture media and scaffolds were incubated for 30 minutes. The media was changed again after 2 hours and 24 hours. On the third day, the culture media of 12 scaffolds were changed with osteogenic differentiation media, while the rest remained in complete culture medium to be served as controls. Media was changed twice a week throughout the study and samples were kept at 37° C. in a 5% CO₂ incubator at 95% humidity. Upon completion of the study, scaffolds were gently washed twice with PBS, and half of the scaffolds were directly stored at −80° C. for RNA extraction to determine the expression of osteoblast and osteocytes markers. The remaining scaffolds were fixed with 10% formaldehyde at 4° C. for 16 h. Fixed scaffolds were washed twice and stored in PBS containing Ca and Mg at 6° C. until use for micro-CT and Cryo-SEM.

### Micro-CT (Micro Computed Tomography)

For the micro-CT, bioink loaded with MSCs after incubation in osteogenic medium and controls were kept in a polystyrene holder submerged in 1×PBS (with Ca and Mg), and data was collected at 55 kV and 188 μA using 0.5 mm aluminum filter and 1° step size.

### Cryo-SEM (Cryo-Scanning Electron Microscope)

For the Cryo-SEM, a thin cross section of each scaffold was flash-frozen in liquid nitrogen and then observed in a low vacuum mode after sublimating the area of interest.

Von Kossa staining scaffolds were cryoembedded and sliced into 14 mm thick cryosections produced using cryomicrotome. The slides were stored at −80° C. until use. Before staining, frozen sections were baked at 40° C. for 20 min, followed by cooling to room temperature for 5 min and then slides were washed in PBS for 2 min.

### Results and Discussion

Alginate is a linear copolymer of β-(1-4)-linked D-mannuronic acid and β-(1-4)-linked L-guluronic acid units, is an anionic polysaccharide. As alginate shows strong chelation in the presence of divalent cations such as Ca²⁺ we selected alginate as a base material in our bioink preparation. Although alginate is a biocompatible material, it is bioinert in nature and therefore does not enable cell adhesion. Therefore, to promote cell adhesion we assessed a combination of either alginate-gelatin, or alginate-collagen bioinks, while crosslinking to alginate was mediated by carbodiimide (EDC/NHS). The EDC was used as a primary crosslinker, which links the carboxyl-acid group of alginate, with the primary amines of gelatin/collagen. Since EDC-mediated crosslinking is only effective at acidic pH, NHS was used to improve the efficiency of crosslinking at pH 7.4. After scaffolds casting or printing, to enhance structural stability, CaCl₂) was used as a secondary crosslinker, to facilitates linkage of α-L-guluronic acid (G-blocks) of one alginate chain with the G-blocks of adjacent chain through Ca²⁺.

First, to optimize the biofabrication parameters at subzero temperatures and its efficacy in creating high-fidelity structures without interlayer mixing and cell death, we used Alginate-gelatin bioink. Thereafter, we used Alginate-collagen bioink for the study of biological properties, considering that collagen is a natural component of tissue, whereas gelatin is produced by denaturing collagen.

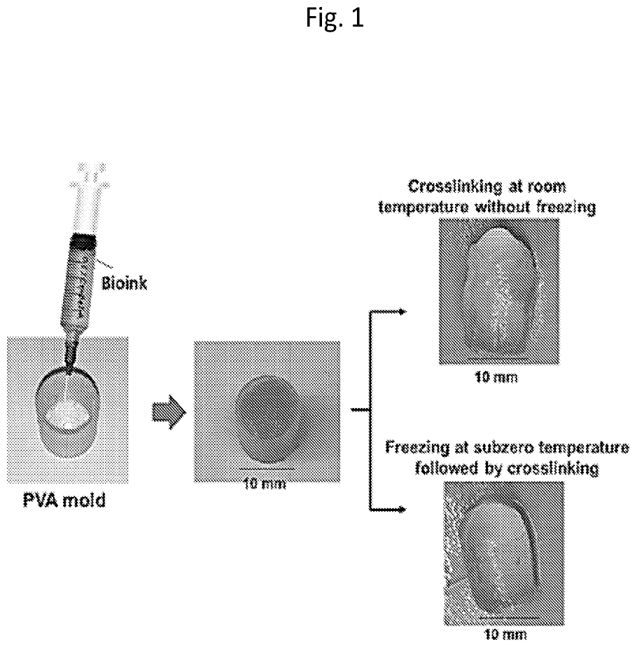

Our novel method of designing scaffolds at subzero allowed the fabrication of well-defined structures using casting as well as extrusion-based bioprinting methods. While our bioink with low viscosity and low storage modulus is good for rapid bioprinting at subzero temperature, it is difficult to create a 3D structure by casting in a mold and removal of structure without damaging it. Therefore, bioink was cast in a 3D printed PVA mold. The use of PVA allowed us to create bigger size scaffolds without structural deformation during the thawing of frozen scaffolds and chemical crosslinking (FIG. 1). As compared to control samples (prepared by crosslinking after casting; no freezing involves), scaffolds prepared by first freezing at −80° C. for 24 h before crosslinking resulted in a 3D structure with dimensional accuracy (FIG. 1).

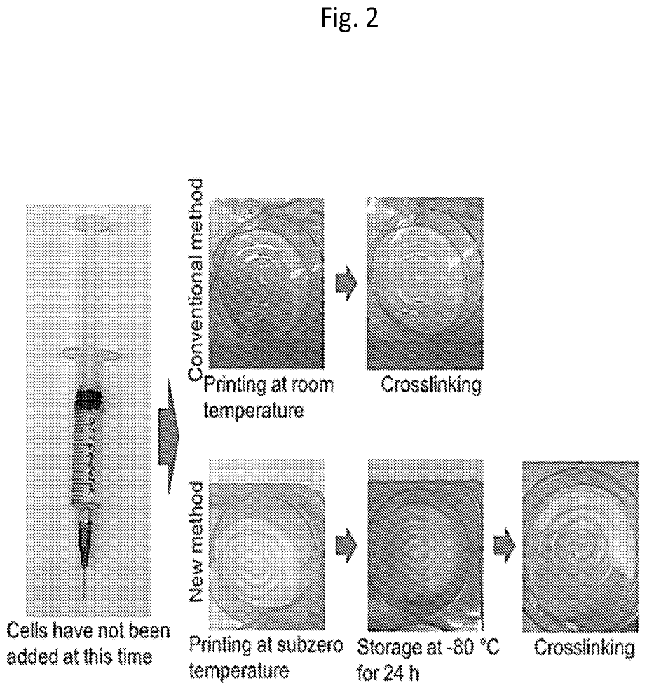

To further validate the efficacy of the designed bioink in creating complex structure, a spiral scaffold was created by manually extruding bioink at subzero temperature, followed by freezing and then crosslinking (FIG. 2).

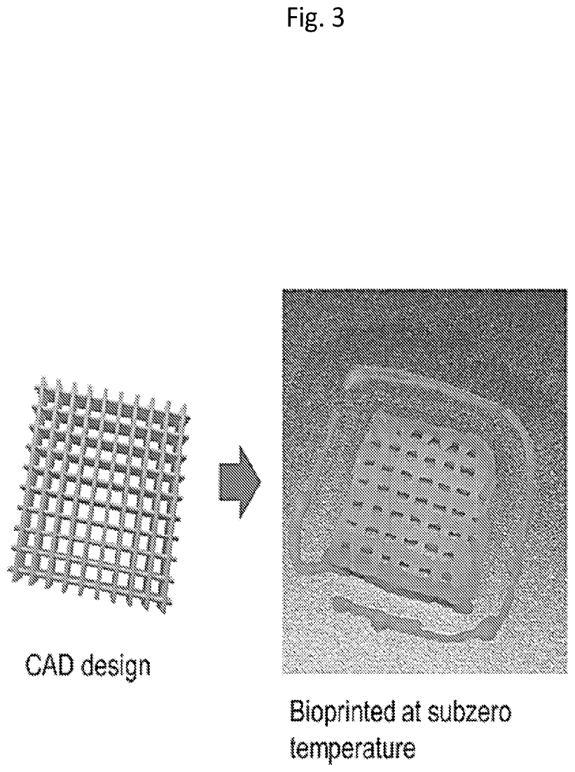

Results showed a well-defined spiral structure after crosslinking. However, designing of similar structure using the conventional method, which involves creating structure at room temperature and crosslinking, leads to the structural deformation in the structure, which can be related to stress created due to crosslinking. The less structural deformation in the structure fabricated at subzero temperature can be related to the crosslinking in its frozen state. Furthermore, a mesh-shaped scaffold based on computer-aided design (CAD) was fabricated at subzero temperature using a bioprinter (FIG. 3).

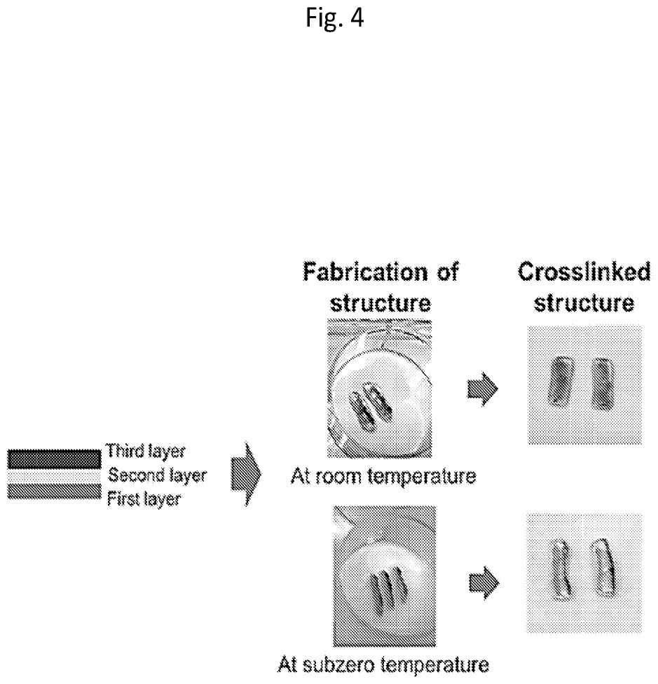

The results confirmed that our novel bioink could be used at subzero temperature to create complex 3D scaffolds based on CAD file. To test the bioink in creating multilayered scaffolds without interlayer mixing, three different 50Alg-50Gel bioinks were prepared-first bioink was without any treatment and therefore visible transparent; the second bioink was prepared by adding red color in it; blue color was added to the transparent colored bioink to make the third bioink. Now, a three-layered structure (without cells) was extruding bioink manually at subzero temperature and at room temperature (conventional method) (FIG. 4). After crosslinking, results showed distinct layers in the scaffolds created at subzero temperature, confirmed the no interlayer mixing. However, excessive interlayer mixing has noted the scaffolds created by the conventional method at room temperature.

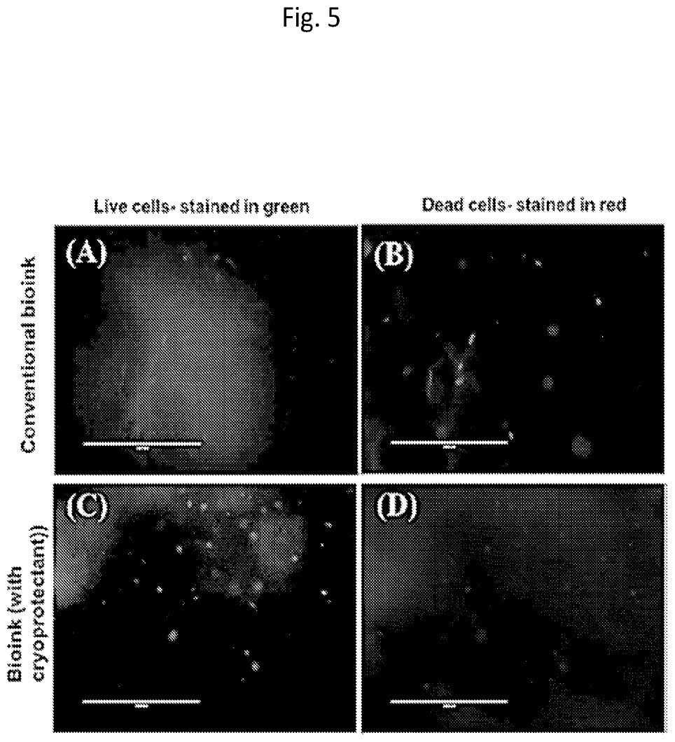

After validating the designed bioink in creating well-defined 3D structures without interlayer mixing, we studied the viability of cells in the scaffold after freezing and storing at −80° C. For this, our novel bioink (with freezing media/cryoprotectant) was compared with conventional bioink (no freezing media or cryoprotectant). The mesenchymal cells (MSCs)-loaded bioinks were cast in a PVA mold and stored at −80° C. for 24 h. After 24 h, scaffolds were thawed in the culture media containing 0.05 CaCl₂). After 2 days of culture, live/dead staining showed the presence of mostly dead cells in the scaffold of conventional bioink (FIG. 5). However, minimal cell death was noted in the case of the scaffold of our novel bioink.

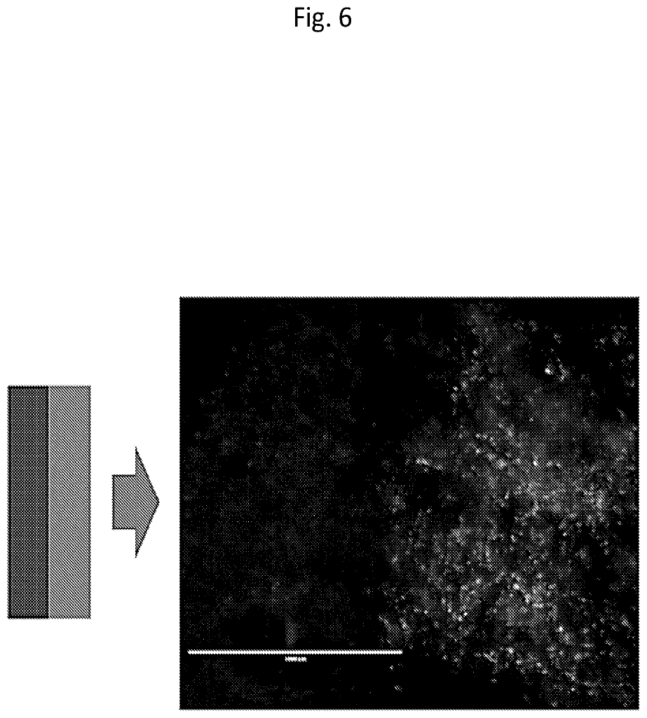

After validating our bioink with cells, the designed bioink was further tested for interlayer mixing with cells by creating 3D structure loaded with cells. As shown in FIG. 6 (with a scale bar of 1000 μm), the designed bioink can be used to fabricate cell-loaded distinct layered with accuracy and without interlayer mixing. It is important to mention that the tissues are characterized by a layered structure of cells with no mixing of layers at the interface. Therefore, we believe that our novel bioink can be used to create such structures and, therefore, open up an opportunity to create complex structures and therefore, to create tissue-mimicking structures.

After confirming the bioink properties in creating 3D structures at subzero temperature without causing cell death and interlayer mixing, we replaced the gelatin in the bioink with collagen to provide a viable substrate for cell adhesion and proliferation. The prepared scaffolds were tested for its swelling behavior, degradability, compressive strength. Furthermore, cell-loaded scaffolds were studied in long-term cell culture conditions in culture media and osteogenic media to study the biocompatibility and to test the ability of the designed bioink in supporting cell differentiation, a key feature required in creating tissue-like structures. Since the composition and mechanical properties of the substrate (bioink/scaffold) can significantly affect the cell-materials interaction and, therefore, the outcomes of the biocompatibility study, therefore, compositional analysis and mechanical properties measurement were carried out using FT-IR and dynamic mechanical analyzer. Because the shear stress created during the extrusion process can cause cell death, therefore, the rheological properties of 50Alg-50Col bioink were also studied using a rheometer.

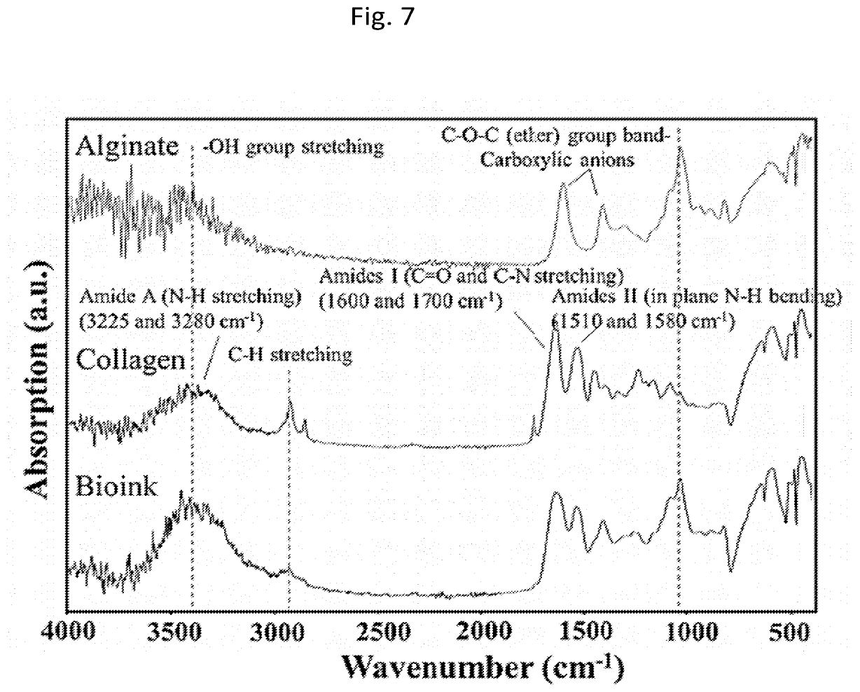

FT-IR was used for the compositional analysis of the 50Alg-50Col bioink. The results were compared with pure alginate and collagen powder (FIG. 7). The absorption peaks at 3450 cm⁻¹, 1618 cm⁻¹, 1440 cm⁻¹, and 1050 cm⁻¹ corresponding to —OH group, —COO—, —COO—, and C—O, respectively, confirmed the presence of alginate. The presence of absorption peaks corresponding to Amide A (N—H stretching, 3225 cm⁻¹, and 3280 cm⁻¹), Amide I (C═O and C—N stretching, 1600 cm⁻¹ and 1700 cm⁻¹), and Amides II (in-plane N—H bending, 1510 cm⁻¹ and 1580 cm⁻¹) confirmed the presence of collagen. The intensity ratio of absorption peaks at 1235 cm⁻¹ and 1450 cm⁻¹ close to 1 is an indicator of the integrity of the triple-helical structure of collagen. We found this ratio equals 1.015 and 1.146 in collagen prepared by the pepsin digestion at acidic pH and collagen in the prepared bioink, respectively.

There are many challenges in the designing of tissue analog structures using conventional bioinks; these are but not limited to slow bioprinting due to high viscous bioink (required to improve the storage modulus) and requirement of crosslinking of individual layer, excessive shear stress-induced on the cells during extrusion, poor shape fidelity, and difficult to make complex structures due to interlayer mixing. The shape fidelity of the printed structure is determined by the storage modulus (G′) and loss modulus (G″), components of the dynamic modulus of a bioink. A higher G′ denotes more solid-like property, while a higher G″ denoted liquid-like property. The storage modulus is simply a measure of the energy required to distort a hydrogel structure. Also, higher storage modulus improves the mechanical strength of the printed structure by enhancing the ability of the hydrogel to store deformation energy and recover in an elastic manner. A higher storage modulus can be achieved by increasing the viscosity of bioink but at the cost of excessive shear stress.

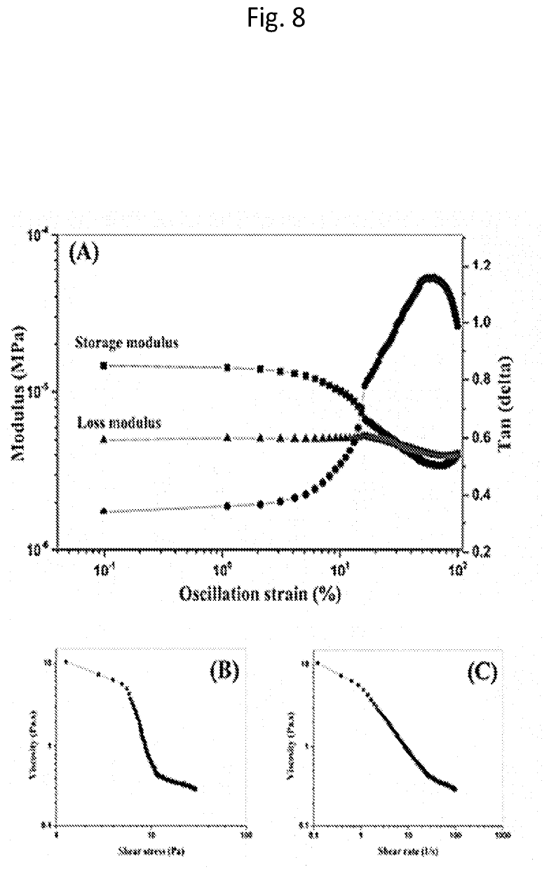

The effect of shear stress can be minimizing by the shear-thinning behavior of bioink under the influence of shear stress. Therefore, to address these challenges, we have designed a novel bioink is a unique combination of biopolymer, cryoprotectant, and growth factors. This bioink allowed us to create the cell-loaded 3D structures at subzero temperature. This new method of fabrication enabled us to create mechanically stable (with high shape fidelity) scaffolds without interlayer mixing by directly freezing the printed layers upon contact with the build plate, maintained at subzero temperature. Importantly, the presence of cryoprotectant in the bioink prevents cell damage during rapid freezing, which was confirmed by live/dead assay. The amplitude sweep at contact frequency showed a significantly low value of storage and loss modulus (FIG. 8a). The amplitude sweep provides information about the viscoelastic behavior of bioink, the value to strain at which bioink change from a more solid-like phase to more liquid-like phase. Although high viscous bioink with high storage modulus is not required in the current method of bioprinting (bioprinting at subzero temperature), it is important to know about the flow initiation and yield stress which give an insight about the printability of the designed bioink. A shear stress ramp is used to determine the yield stress of bioink. The plot between shear stress and viscosity provides the value of stress at which bioink will start flowing during extrusion (FIG. 8b). We observed a decrease in the viscosity of bioink with the increase in the shear stress. At ˜1 Pa shear stress, the viscosity of bioink was 10 Pa·s, which decreases linearly until ˜5 Pa, and after that, a steep decrease in the viscosity was noted due to the yielding of bioink, which confirmed the printability of the designed bioink. It is important to mention that our bioink demonstrated a significantly lower value of yield stress (˜5 Pa) than the existing bioinks (70-200 Pa) (Paxton et al., Biofabrication, (2017), 9:044107). Our bioink also showed a shear-thinning behavior, characterized by a decrease in viscosity with an increase in the shear rate (FIG. 8c). A low viscosity bioink with a lower yield point and shear thinning behavior minimizes the possibility of shear stress-induced cell death. Also, it allows rapid bioprinting due to flow initiation at low shear stress.

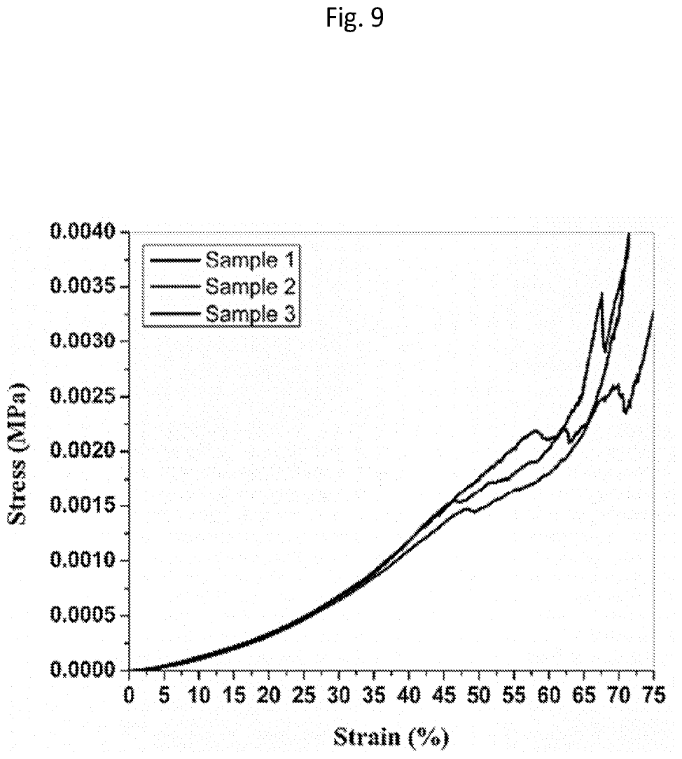

Structural resilience is required for the safe handling of scaffolds and for the implantation. Compression testing at 37° C. in submersion condition (in 1×PBS) showed a compressive strength of 2.520±0.235 kPa. The stress-strain curve showed an increase in the stress with a yielding before reaching to a fracture point (FIG. 9). After the fracture, an increase in the stress was noted as a result of densification can be related to the closure of pores at high compressive loading, a feature of polymeric materials under compressive loading. The calculated value of yield strength and bulk elastic modulus of scaffolds were 1.720±0.143 kPa and 0.069±0.015 kPa, respectively. At the yield point, the linear (elastic) region of the graph terminates, and after this, the material behaves plastically, and elastic recovery is not possible after this point. Important to mention that the composition and scaffolds in this study have been designed to only show the efficacy of this new method of biofabrication by freezing the printed layers at subzero temperature to get a structure of high fidelity without cell death and interlayer mixing. Based on future applications, the strength of scaffolds can easily be increased by increasing the weight percent of alginate and collagen in the bioink and/or changing the concentration of CaCl₂) as well by increasing the time of crosslinking. Furthermore, a stronger scaffold for load-bearing tissue engineering applications can be designed by constructing a hybrid structure. To create a hybrid structure, bioink filled in a mechanically strong porous scaffold, followed by crosslinking (Kumar, et al., Journal of Tissue Engineering and Regenerative Medicine, (2018), 12:1133-1144). The ease of alteration in the mechanical and physical properties makes the designed bioink suitable for various tissue engineering applications ranging from neural, cardiac, skin, and muscle. However, a detailed study is required to validate the use of the designed bioink for various tissue engineering applications.

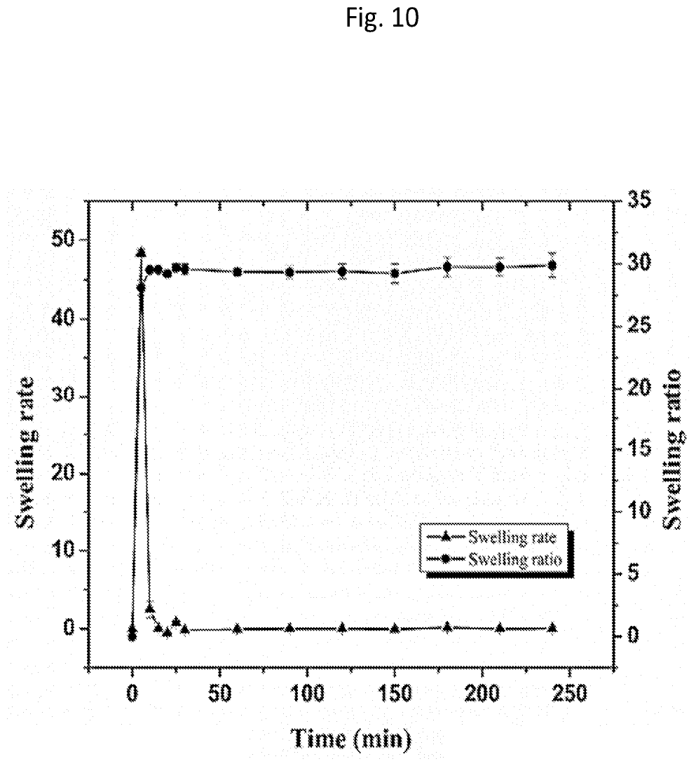

The dimensional tolerance is an important factor in biomedical applications, which can be affected by the swelling of scaffolds during application. We noted a significantly high swelling rate (˜48 mg/min) in the first 5 minutes, and then it decreased to ˜3 mg/min in the next 5 min when a freeze-dried cylindrical-shaped scaffold (diameter×length: 10 mm×5 mm) submerged in 1×PBS (with Ca, Mg) at 37° C. (FIG. 10). After 10 min, we did not notice any change in the swelling rate. The measured value of equilibrium swelling ratio and percentage equilibrium water content were 29.531±0.481 and 96.723±0.052, respectively.

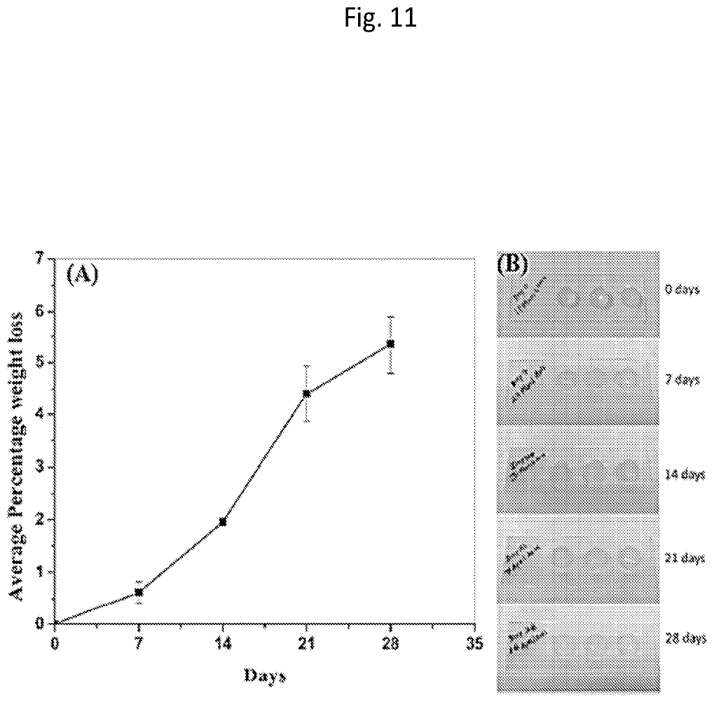

The dissolution study conducted for 28 days in 1×PBS (with Ca, Mg) at 37° C., 5% CO₂, and 95% relative humidity showed a time-dependent loss in weight of scaffolds, which confirms the biodegradability of the designed bioink (FIG. 11a). Furthermore, no change in pH was noted during the course of the study. During the dissolution study, no dimensional change in the scaffolds due to swelling was observed, which confirms the structural stability of the scaffolds (FIG. 11b). The designed bioink can be used for loading and delivery of drugs and growth factors at the defect site for the faster regeneration of damaged tissues. In this context, dissolution behavior is a critical parameter required for the controlled delivery of drugs of growth factors. FIG. 11a showed a steady dissolution profile with no abrupt increase in the dissolution of scaffolds. Such a controlled dissolution of the scaffolds prevents the burst release of drugs and thus can prevent the pharmacologically dangerous effects (Costa et al., European journal of pharmaceutical sciences, (2001), 13:123-133).

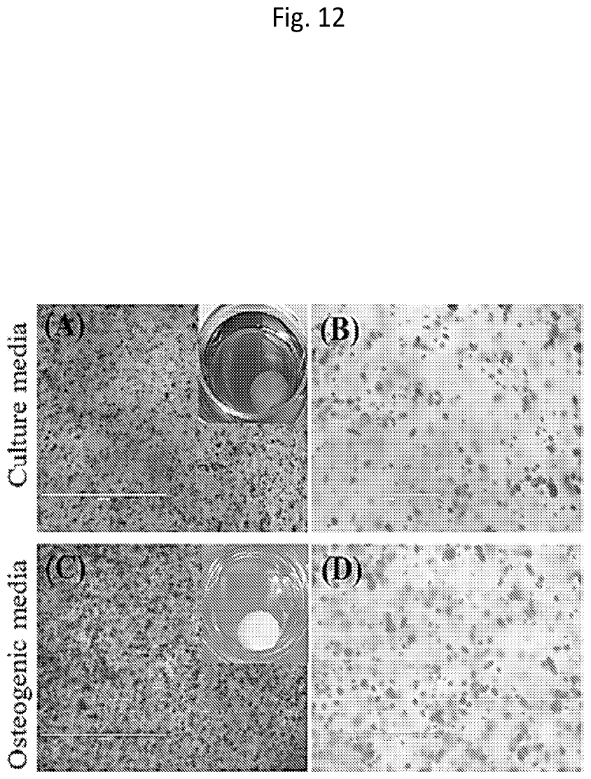

Scaffolds used in the differentiation study are characterized by 3D cylindrical-shaped structures with uniform cell density. The light microscopy of the scaffolds kept in culture and differentiation media revealed the tissue-like structure with cell in high density of cell in three-dimensional space (FIG. 12).

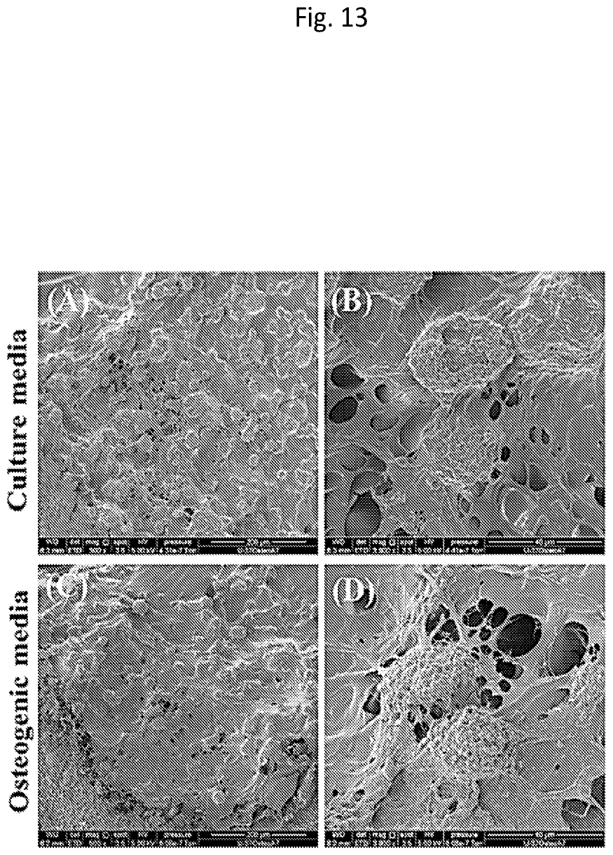

After 70 days of culture, fixed scaffolds were analyzed to see the presence of Ca—PO₄ crystals (bone mineral) deposited by the osteogenically differentiated MSCs. CryoSEM showed the presence of cells attached to the bioink matrix, and no dead cells were visible (FIG. 13a,b). Careful observation revealed a difference in the size of cells in culture media and differentiation media. Cells in differentiation media were relatively of bigger size when compared with cells in culture media (FIG. 13c,d).

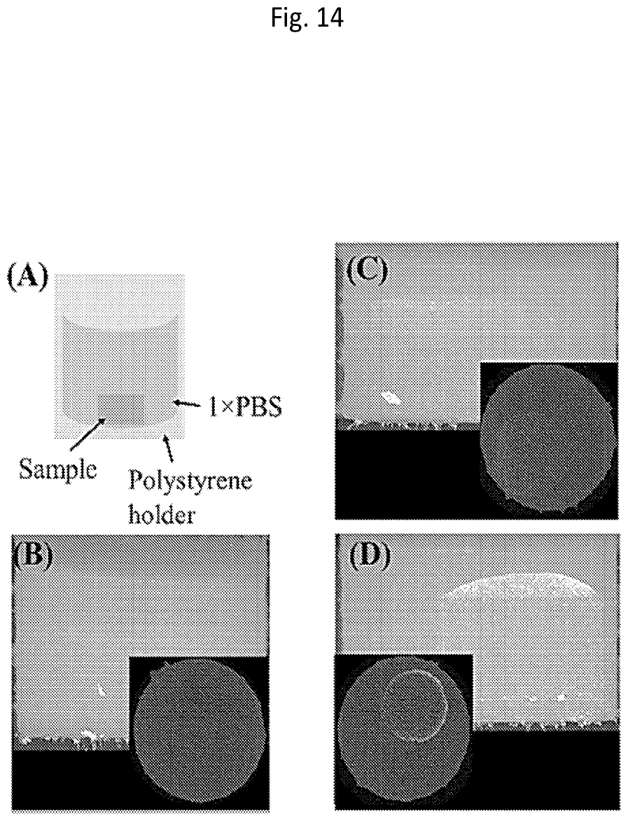

Furthermore, the microCT confirmed the presence of Ca—PO₄ crystals (white in color) in both scaffolds kept in cell culture media and osteogenic differentiation media (FIG. 14c,d). However, as compared to culture media, the density of Ca—PO₄ was higher in osteogenic media. Importantly, as shown in the inset images, deposition of the bone mineral was higher on circumference than the center. This may be due to higher metabolic activity of cells or osteogenic differentiation of cells due to efficient transport of nutrients near the surface (circumference) as compared to the cells growing inside the scaffold.

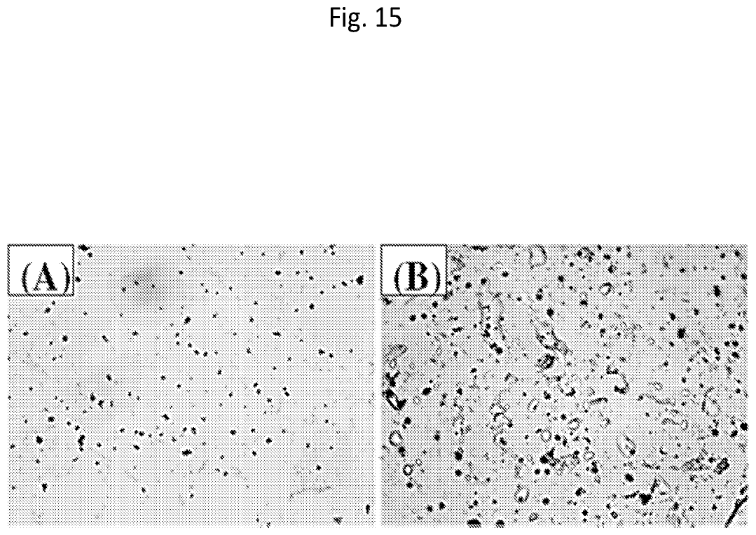

The von Kossa staining of cryosectioned samples confirmed mineralization of both scaffolds kept in culture media and osteogenic media (FIG. 15a,b). However, the density and size of the deposited mineral were higher in the case of osteogenic media than culture media (FIG. 15c,d). Overall, this study disclosed the effectiveness of our novel bioink in creating complex cell-loaded structures without interlayer mixing and cell death. Also, the designed bioink can be used as a substrate to grow the tissue-like structure, supported by cell viability and differentiation assay data.

### Conclusion

The new alginate-based bioink with cryoprotectant can be used to create multilayered structures of different cells to mimic the tissue structure and function. The designed high-fidelity structures at a subzero temperature can be stored at −80° C. without causing cell death and be revived easily without losing cell functionality. The designed bioink can be used to create 3D structures either by casting or by bioprinting. The new bioink and method of scaffold fabrication present a major advancement in creating of tissue-mimicking structures. The future development of this technology may allow the generation of off-the-shelf clinical-size living grafts for reconstructive surgeries.

### Example 2—Development of Three-Dimensional Cell-Laden Bioink Scaffolds for Treating Bone Loss

More than two million patients per year undergo repair of large bony defects with costs higher than $2.5 billion. Long-term space missions pose additional concerns as microgravity-induced bone loss increases the risk of bone fractures and premature onset of osteoporosis in astronauts. This may impose a significant challenge to manned missions to Mars and future deep space travel plans, affecting astronaut quality of life during and after space missions. Prefabricated bioengineered bone may serve as means to mitigate bone deterioration and provide a more viable solution for bone repair and faster recovery. Human mesenchymal stem cells (MSCs) hold great potential for bone regenerative therapies, and bioengineering applications. However, MSCs have been shown to have reduced potential to form bone in microgravity. Furthermore, methods are still required to create appropriate three dimensional (3D) structures that properly mimic bone structure. To overcome the effects of microgravity, we developed in our laboratory a new approach for 3D bioprinting, which uses solid free-form fabrication for the precise placement of layers of stem cells with biological growth factors in freezing conditions using a specialized BioInk (CryoBioInk), mimicking the complex architecture of the bone tissue. Our preliminary data show that we can efficiently generate mineralized bone cells in our 3D printed scaffolds. This is a critical barrier in bone regenerative therapies, as methods to increase the differentiation of MSCs are still needed. To develop a bone replacement solution for long term missions to Mars, we propose to study the effect of long-term cryogenic storage and the ability of our stem cells to generate correct bone architecture in microgravity.
1: Define the effect of our CryoBioInk on MSC survival and bone differentiation after long-term cryogenic storage. Our data indicate that MSCs in our CryoBioInk efficiently survive the −80° C. cryogenic storage and proliferate upon thawing. We will assess MSC survival and bone differentiation in our conventional 3D-printed, bioengineered bone scaffolds following long-term cryogenic storage in liquid nitrogen and after long-term deep cryogenic storage in −80° C. by the following: a. Determine the effect of our CryoBioInk on cell viability and proliferative capacity. b. Define the efficiency of bone differentiation in our innovative scaffolds. c. Assess cell migration and distribution throughout the scaffold. d. Assess the bioengineered bone quality, mechanical strength and biodegradation. We expect our CryoBioInk will support the differentiation potential of MSCs and allow the transport and long-term storage of the resultant tissues on the scale required for bone regenerative medicine and for the repair of bony defects in reconstructive surgery.
2: Determine the effect of our CryoBioInk on gene expression, survival and bone differentiation potential of MSCs in microgravity. Devices will be launched and grown onboard the ISS national laboratory after cryogenic storage. To study the effect of microgravity on long-term potency and the ability to form bone, we will: a. Determine the effect of our CryoBioInk in 3D scaffolds on undifferentiated MSCs viability and proliferative capacity following cryogenic storage and growth in microgravity. b. Assess the effect of our CryoBioInk following cryogenic storage on osteogenic differentiation of MSCs in microgravity. c. Define the effect of CryoBioInk on osteogenic gene regulation in microgravity compared to those grown in our laboratory on Earth. The data obtained in these analyses will be an instrumental step in optimizing the procedures for bioengineering of patient-specific off-the-shelf bone replacement devices.

### Project Description

During a 6-month space mission, about 1-1.5% of bone density loss per month is observed in astronauts, exposing them to increased risk for fractures. It is unclear if this decline in bone mass will progress or accelerate in longer space missions or in lower gravity environments such as those on Mars or other planetary bodies, potentially creating significant bone-health related issues. The limited medical capabilities and lack of medical facilities to repair a fracture in a crew member away from Earth may lead to more medical complications with higher morbidity, loss of life and may jeopardize mission objectives. The microgravity environment may impair bone healing, elevating the risk for bone fractures, non-union of the fractured bone, and earlier onset of osteoporosis. Therefore, a successful long-term space travel mission must include means to mitigate bone deterioration and provide the most efficient solution for bone repair and expedient recovery.

Musculoskeletal-related injuries or diseases are a common and costly problem. Bone injuries become more acute in the ever-growing elderly population, due to delayed bone healing and osteoporosis-related complications, with more than two million patients per year undergoing repair of large bony defects with costs higher than $2.5 billion (8). Specifically, in older patients, the healing of bone defects is difficult due to decreased bone density. There are two primary options currently available for bone reconstruction to restore lost bone and its complex structure: I) bone harvested from the patient by additional surgeries, called autografts, and II) bone from cadaver donors, called allografts. However, both options have major limitations and drawbacks. Autologous bone grafts typically require a second operative site to harvest bone from the same patient, which is costly and risks significant morbidity to the patient. Microvascular free tissue bone grafts also require a second surgical site with consequent donor site morbidity and are often contraindicated in patients with vascular disease and diabetes. Furthermore, the amount of bone that can be harvested may be limited. Allografts from cadavers are expensive, carry the risk of infectious disease, and require freezing, resulting in decreases in mechanical strength, osteoinduction and osteogenesis. Thus, bone bioengineering offers an attractive alternative to existing methods and may have tremendous benefits for adult individuals with bone pathologies.

Completion of this application will generate next-generation, off-the-shelf 3D-bone grafts. These clinical devices can be kept in stasis and in cryogenic conditions for use in bone reconstructive surgeries. Our mesenchymal stem cells are non-genetically manipulated and the implant fabrication and culture conditions will set foundations for the procedures required for manufacturing clinical-grade implants.

Bone bioengineering relies on three main components: stem cells, scaffolds, and growth factors. The growth and differentiation of stem cells onto a 3D scaffold in the presence of growth factors promote the subsequent formation of bone in vitro. Adult mesenchymal stem cells (MSCs) have become a promising stem cell source for bone tissue engineering due to socio-ethical acceptance and the ability to obtain autologous stem cells from patients. MSCs can be isolated from multiple tissues, such as bone marrow adipose tissue, liver, umbilical cord blood, and tonsils.

We have developed a novel procedure and material to 3D print scaffolds mixed with MSCs to mimic bone structure and the tissue microenvironment. We further developed a CryoBioInk that provides the cells with 3D attachment support and allows flash freezing as cells are printed or infused into the more solid 3D printed scaffolds. We will use MSCs incorporated into the CryoBioInk to generate a bone reconstruction/repair device. Our approach may have tremendous benefits for individuals with bone pathology, whether from disease, tumor, or trauma. The designed bone grafts will provide a viable solution for repair and faster healing of bone defects. We hypothesize that our 3D printed structures with stem cells-loaded (CryoBioInk) will ensure survival, support cell attachment and differentiation of the adult stem cell-derived bone tissues after prolonged periods of freezing. Importantly, the frozen CryoBioInk structures can be easily transported to space without significant damage to the materials or the embedded cells. We expect that the CryoBioInk will act as a substrate for cells to attach, grow, and differentiate to form bone in microgravity and therefore will provide an efficient means to generate bone replacement that will survive in long term storage and the conditions in space travel.

A 3D clinical-scale ex-vivo bone model to study the effects of microgravity and drug design. Bone density is reduced at a rate of about 1-1.5% per month in astronauts during a 4-6-month space mission, exposing them to increased risk for fractures. This decline in bone mass may progress further in longer space missions, and it is still unknown if a partial gravity environment such as on Mars or other planetary bodies will create additional bone-health related issues. The microgravity environment may impair bone healing, elevating the risk for bone fractures, non-union of fractured bone, and earlier onset of osteoporosis. The outcomes of this study will also yield a 3D clinical-scale bone model which can be used as an ex-vivo model for the study of the effect of microgravity on bone pathology and can be a useful tool for drug design to prevent or decrease bone density loss in astronauts and other bone related diseases. Our study may provide a tissue engineering platform to study the impact of space travel and microgravity on the decline in osteogenic potential as well as a testing platform for potential treatments for osteoporosis.

A unique bioreactor and bioreactor chamber design. The experiments in our laboratory will include the use of our unique bioreactor design. We will compare scaffold-grown cells in our dynamic bioreactor to cell growth in the same bioreactor chambers in static conditions. We used a Cole-Parmer Masterflex peristaltic pump which we chose for two primary reasons: first, the pump generates a flow of 3 ml/min which is comparable to flow rates in previous studies utilizing perfusion bioreactors. Second, because the pump is peristaltic, the flow generated is pulsatile rather than continuous. This is important as pulsatile flow has been shown to better augment bone formation compared to steady flow. The growth chamber of our bioreactor consists of platinum-cured silicone tubing to improve the strength of the tubes, and provide intrinsic antimicrobial properties. The bioreactor chamber is made of polystyrene, the same material used for tissue culture dishes and allows for visibility of the scaffold. Moreover, our bioreactor is designed to allow the growth of the scaffolds in suspension conditions and to be fed with medium from the inside and outside. This represents another major innovation as it prevents the scaffold from pushing against the side of the growth chamber and allows maximal media exposure on all sides of the scaffold.

Scaffold design based on functional anatomy and MSC growth and differentiation considerations. Current scaffold designs still require means to promote cell migration throughout the scaffolds. The limited supply of nutrients and oxygen within the scaffold further leads to the migration of cells toward higher nutrient areas resulting in cells coating periphery of the scaffolds, but limiting cell growth inside and throughout the device. Based on our combined expertise in stem cell research, biomaterial and bioengineering engineering, and bone biology, we designed novel cannulated scaffolds to mimic the architectural complexity of bone, allowing for the flow of nutrients within the device and promoting more homogenous stem cell growth. Our unique cannulated scaffold design allows us to inject cells inside the scaffold during seeding. It also allows connection of the scaffold to a tube outside the bioreactor growth chamber so that the scaffold is nourished through the main channel. Our design thus allows the scaffold to be immersed in medium from the outside and to be nourished with medium from the inside.

### Research Plan

We recently reported a novel approach that allows us to harvest millions of adult human stem cells from a small tonsillar biopsy of less than 0.6 grams of tissue in an outpatient procedure, without the need for hospitalization or general anesthetics. Importantly, 1 gram of tonsillar tissue yields at least 10⁴ fold more viable MSCs than adipose or bone marrow sources (Table 1), and can be further expanded in tissue culture. We further developed the conditions to seed our cells onto 3D printed scaffolds and efficiently differentiate the cells into osteoblasts and osteocytes (bone cells). These cells are easy to harvest in high yield and may provide a translational approach to harvest stem cells pre-flight from astronauts to generate personalized, autologous grafts. For translational purposes and to allow the development of patient-specific medical devices, which can be generated in preparation for long-term space missions, we will compare these cells to bone marrow MSCs.

TABLE 1
Tonsillar biopsies yields multiple orders of magnitude
more MSCs compared to other tissue sources.

| | Yield | |
|---|---|---|
| Tissue Source | Stem cells/gr tissue | Stem Cell Quality |
| Bone marrow | 1500-3000 | High |
| Adipose tissue | 1000 | Low |
| Dental pulp/baby teeth | <100 | High |
| Umbilical cord | 2.5 × 10⁵ | High |
| Tonsil (with our protocol) | 3 × 10⁷ | High |

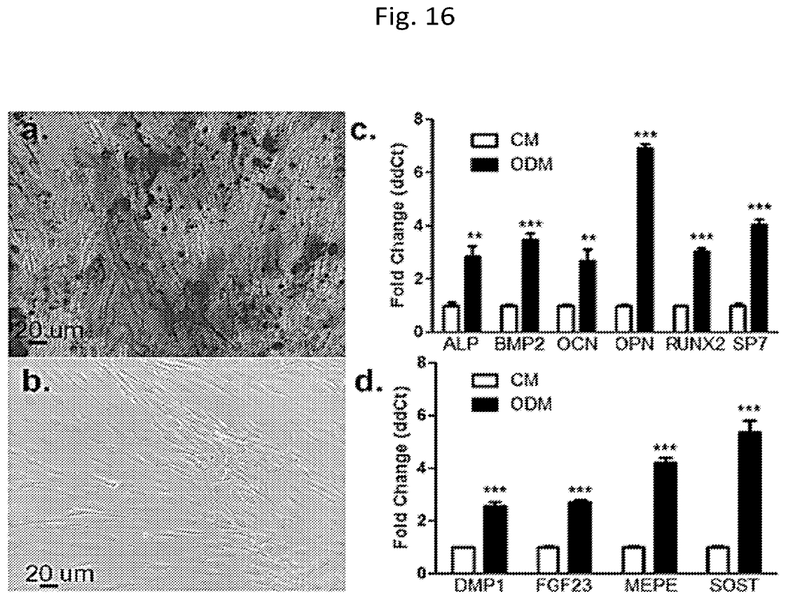

Osteogenic differentiation of MSCs. To define the osteogenic differentiation potency of our tonsillar biopsy MSCs, cells were grown in osteogenic differentiation medium for 21 days. Following osteogenic induction, the morphology of our MSCs dramatically changed from a fibroblastic phenotype to the expected, more flattened morphology with multiple cell extrusions. Our results demonstrate that differentiated cells are positively stained for Alizarin Red (FIG. 16a, b), suggesting osteogenic differentiation and extracellular calcium accumulation. Control MSCs grown in complete medium were negative (FIG. 16). Furthermore, our RT-qPCR results validate osteogenic differentiation, as we observed a significant upregulation in the osteoblast markers: Alkaline phosphatase (ALP), Bone morphogenetic protein 2 (BMP2), Osteocalcin (OCN), Osteopontin (OPN), Runt-related transcription factor 2 (RUNX2), and Osterix (SP7) (FIG. 16c). Remarkably, following osteogenic differentiation, our results also indicate a significant upregulation of more mature osteocyte markers such as Dentin Matrix Protein 1 (DMP1), fibroblast Growth Factor 23 (FGF23), Matrix Extracellular Phospho-glycoprotein (MEPE), and Sclerostin (SOST) (FIG. 16d). Our data suggest that MSCs may form osteoblasts and further suggest they progress to form mature osteocytes in culture. Our data demonstrate our ability to maintain MSCs and to induce their differentiation to the bone lineage.

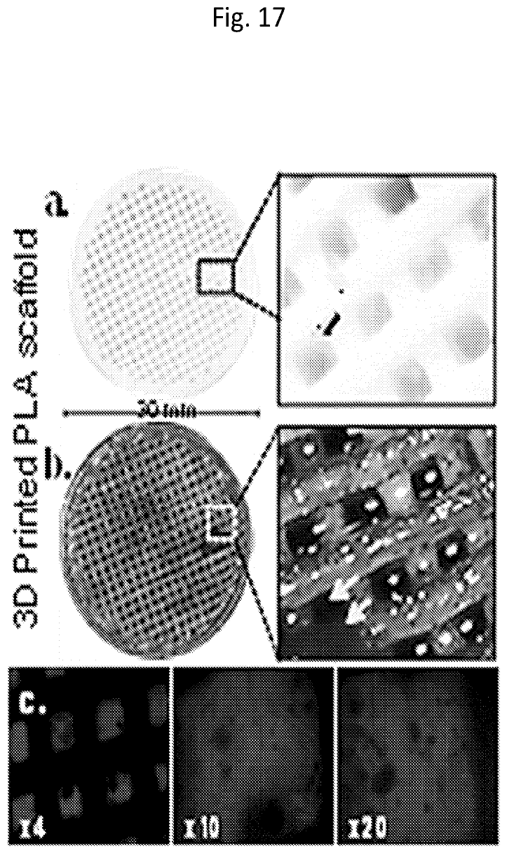

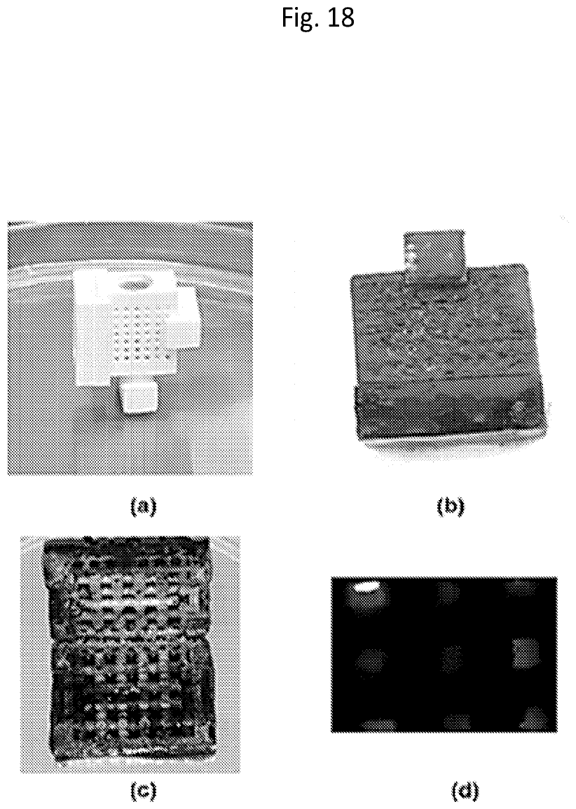

MSC derived bone cells in 3D scaffolds. Next, we designed and tested a 3D-scaffold of polylactic acid (PLA) using a fused deposition modeling (FDM)-based 3D printer. We selected PLA for several reasons: (i) PLA is an FDA approved material for many existing applications including bone fillers and arterial scaffolds. (ii) It allows the design of biocompatible scaffolds with a compressive strength to provide the structural support necessary during the reconstruction of bone defects, and (iii) PLA is slowly bioabsorbable, which provides functional and mechanical support when implanted, yet, over 4 years, the scaffold is replaced by native bone tissue⁴. Multiple studies have evaluated different pore sizes and have shown that a mean pore size of around 300-500 μm displays enhances cell proliferation and differentiation throughout the scaffold due to enhanced transport of oxygen and nutrients. Our 3D printed PLA scaffolds were seeded with MSCs and cells were differentiated into osteoblasts in static conditions. Our data show that MSCs can efficiently differentiate to osteoblasts in our 3D scaffolds (FIG. 17, FIG. 18).

MSC-culture on the 3D printed scaffolds and in vitro bone formation. In preparation for this application, we printed a scaffold of clinically relevant dimensions: length: 18.5 mm, width: 13.5 mm, height: 10 mm. The central channel with interconnected pores of different sizes was found to promote the seeding of high density (˜5×10⁶ cells/per scaffold) MSCs in the scaffold (FIG. 18a). The results showed a homogenous cell distribution in the scaffold (FIG. 18b, c). We noted the migration of MSCs to the pores of scaffold and their survival and proliferation in long term static culture. The 35-day culture of MSCs on the scaffolds in osteogenic media showed a high yield of differentiated MSCs and deposition of extracellular calcium by these cells to form bone on the 3D printed PLA scaffolds (FIG. 18 b-d).

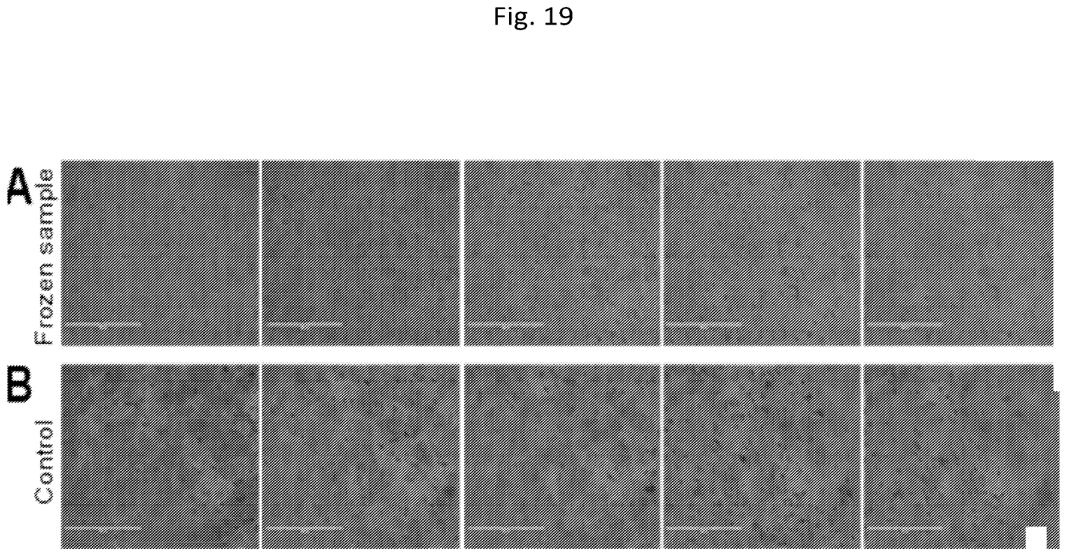

Our CryoBioInk. We have successfully designed a CryoBioInk for the construction of MSC-loaded scaffolds that can be directly printed in cryogenic conditions. These novel scaffolds can be stored at −80° C. for an extended period and can be directly thawed in culture media without significant loss of cell viability. The frozen scaffolds can then be incubated in an osteogenic media for the induction of MSC bone differentiation. We synthesized our bioactive CryoBioInk by mixing alginate and gelatin in deionized water at room temperature using carbodiimide-induced crosslinking. For preliminary studies, the scaffolds were designed by casting CryoBioInk loaded with MSCs in a Polyvinyl alcohol (PVA) water soluble mold, followed by crosslinking. The MSCs were pre-stained fluorescently red using Cell Tracker Red CMTPX which allows us to visualize the cells within the scaffolds and track cell viability. The cells were mixed into freezing medium and the hydrogel, and scaffolds were flash frozen and stored at −80° C. for the desired period. For cell viability evaluation, the frozen scaffolds were immersed in culture media at 37° C., washed after 10 minutes and incubated in media in a CO₂ incubator. The PVA mold was dissolved and the media was changed after 2 hours and again after 24 hours. For cell maintenance, the medium was replaced every three days. For the control samples, scaffolds were directly stored in culture media after the crosslinking, in the CO₂ incubator at 37° C. After 8 days, viable cells were visualized under a fluorescence microscope. Our data show a homogenous distribution of viable MSCs throughout the CryoBioInk matrix (FIG. 19). The images were captured at different depths and their vertical stacking confirmed the presence of cells and their homogenous distribution in three-dimensional (3D) space. Interestingly, the morphology of cells in the frozen samples was similar to the control samples. The red fluorescence of the CMTPX as well as the cell morphology in both frozen and control samples confirmed cell viability, attachment and proliferation in the designed CryoBioInk scaffold.

Our data indicate that CryoBioInk preserves the viability of the cells in 3 dimensions in cryogenic storage. This will further allow the secure transportation of the tissue engineered grafts in frozen conditions from Earth to the ISS National Laboratory. Successful completion of this project will yield bone replacement devices that can be kept frozen in future long-term space flights as means to repair non-union bone fractures. These devices and procedures will have additional significant applications in tissue 3D printing and reconstruction.

### Experimental Design and Methods

1: Define the effect of our CryoBioInk on MSC survival and bone differentiation after long-term cryogenic storage. Our data indicate that MSCs in our CryoBioInk efficiently survive the −80° C. cryogenic storage and proliferate upon thawing. We will assess MSC survival and bone differentiation in our conventional 3D-printed, bioengineered bone scaffolds following long-term cryogenic storage in liquid nitrogen and after long-term deep cryogenic storage in −80° C. by the following sub-aims: a. Determine the effect of our CryoBioInk on cell viability and proliferative capacity. b. Define the efficiency of bone differentiation in our innovative scaffolds. c. Assess cell migration and distribution throughout the scaffold. d. Assess the bioengineered bone quality, mechanical strength and biodegradation. We expect our CryoBioInk will support the differentiation potential of MSCs and allow the transport and long-term storage of the tissues on the scale required for bone regenerative medicine and for the repair of bony defects in reconstructive surgery.

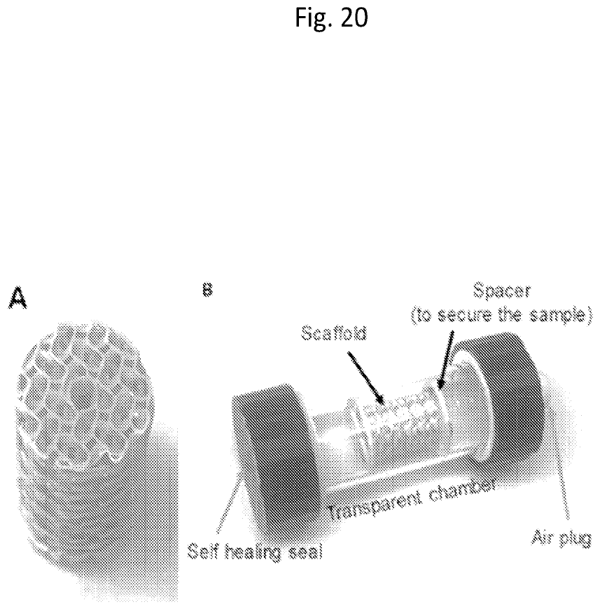

3D biodegradable scaffolds of biocompatible materials with interconnected pores aptly mimic the extracellular matrix properties of bone. This is important because the 3D printed scaffolds with predefined architectural complexity can provide the required mechanical properties for the stability of resultant bone with room for vascularization and tissue ingrowth. Based on our joint expertise in stem cell research, bioengineering, and material engineering, we have designed an innovative bioreactor chamber (FIG. 20). We further developed an effective scaffold structure and composition to promote stem cell growth, homogenous distribution and differentiation to bone. 3D printed scaffolds with predefined shape, porosity, and mechanical properties, along with our CryoBioInk allow for the flash-freezing and storage of the 3D devices and facilitate cell viability, migration and incorporation throughout the scaffold, promoting cell survival and bone differentiation. This will allow us to generate off the shelf clinical-size living bone grafts for bone reconstructive surgeries and storage in long-term space missions.
1a: Determine the effect of our CryoBioInk on cell viability and proliferative capacity following cryogenic storage. MSCs from at least 3 donors will be cultured in growth media in humidified air with 5% CO₂ at 37° C. Cells will be pre-stained with a long-term cell viability and proliferation tracker “Qtracker 625” stable for weeks in the daughter cells. PLA 3D printed scaffolds will be placed into a PVA mold and loaded with MSCs in CryoBioInk, flash frozen and transferred to −80° C. overnight. The next day, scaffolds will be transferred and connected to the growth chamber of the bioreactor (FIG. 20) and will be stored in cryogenic conditions at increasing time intervals from one week, one month and up to one year in −80 C and compared to equivalent scaffolds in liquid nitrogen. For thawing, briefly, the scaffolds will be placed in cell-thawing media for 10 minutes. The medium will be replaced with culture medium to remove the PVA mold and the medium will be replaced after two hours and fresh medium will be added for 24 hours to attain full hydration. Then, osteogenic medium to promote bone growth will be used and replaced every three days. As controls, we will use isogenic cells grown in the same conditions in growth medium without osteogenic factors. Additionally, upon thawing, we will repeat these experiments to compare cells grown in the bioreactor chamber in static conditions to samples grown in a dynamic environment in a bioreactor using a peristaltic pump. Media will be pumped in the bioreactor and will be replaced once a week for up to 21 days and cells seeded on scaffolds in osteogenic medium in static conditions. We will determine the effect on cell viability of prolonged 3D cultures in our bioreactor by following Qtracker 625 and compare our results to MTT and LDH assays, and by cell counting and calculating population doublings. Additional scaffolds will be taken for histological analyses. We expect that cells grown in the dynamic environment in the bioreactor will show higher viability and better distribution throughout the scaffold compared to scaffolds populated with cells in static conditions.
1b: Define the efficiency of bone differentiation in our innovative scaffolds. We will determine cell differentiation potential into osteoblasts by monitoring alkaline phosphatase activity and through histochemical analysis staining by Alizarin red, a marker for bone mineralization. ALP is an enzyme that is found in a high concentration in bone forming cells and is an established method to evaluate osteogenic differentiation. RNA will be taken to RT-qPCR analysis to analyze expression of osteogenic markers, such as Col1a1, Runx2, Osterix/Sp7, Osteocalcin, Alkaline phosphatase, BMP2, TGFB2, osteopontin (OPN) and osteoahderin. Further differentiation to osteocytes will be assessed by the osteocyte markers: FGF23, DMP1, SOST and MEPE. To demonstrate our findings via RT-qPCR are conserved on the protein level, data will be validated by immunoblot and immunostaining. For positive controls, we will use osteosarcoma cell lines which are positive for these markers. We predict that cells grown in the bioreactor conditions will demonstrate increased bone differentiation capacity throughout the scaffold and therefore will show increased expression of bone markers. These analyses will determine the effect of our scaffold on the efficiency of osteogenic induction and cell differentiation.
1c: Assess cell migration throughout the scaffold. To assess cell migration in the scaffold, we will seed cells in two phases. On day one, MSCs will be permanently marked by a green fluorescence long term cell tracker dye (Qtracker 525) and seeded in CryoBioInk on one side of the scaffold in static conditions. The next day, the scaffold will be thoroughly washed, a-cellular CryoBioInk will be casted and the other opposite of the scaffold seeded with cells marked with long-term red fluorescence cell tracking dye (Qtracker 625), and then incubated for an additional 24 hours. The scaffold will then be washed and assessed on both sides to ensure the separation of the Green and Red cell populations. Next, the scaffold will be inserted into the bioreactor. Cells will be allowed to grow for up to 3 weeks. As a control we will use cells without osteogenic medium in the bioreactor and cells grown in the osteogenic medium in static conditions. We will assess cell migration inside the scaffold by fluorescence confocal microscopy. Additional scaffolds will be taken for X-ray imaging and by micro-CT analyses (Skyscan 1172 micro-CT, Bruker) to assess calcium deposition homogeneity and bone density throughout the scaffold, compared to scaffolds grown in static conditions. This technique will also allow us to determine and calculate the fraction and composition of the newly formed bone out of the total volume of the scaffold. Prior to cell seeding, we will pre-scan our acellular scaffolds by microCT (45 kV, with 10-micron voxel size). Then, following the completion of the experiment, we will rescan the scaffold and quantify the change in mineral accumulation and scaffold porosity. Additional engineered bones will be fixed in paraformaldehyde and paraffin embedded for processing. 5 μm sections will be made and taken for staining procedures, including H&E and Masson trichrome staining to assess the growth and differentiation of bone cells throughout the scaffold. The histological analysis will allow us to determine cell phenotype, as well as quantify viability, fibrosis, apoptosis and necrosis. These experiments will demonstrate the ability of cells to migrate and grow throughout the scaffold.
1 d. Assess the bioengineered bone quality, mechanical strength and biodegradation First, since surface topography (microstructural features) can affect cell attachment, polarization and proliferation, triplicate samples of acellular scaffolds, scaffolds with undifferentiated cells and scaffolds following procedures as described in sub-aim 1a, will be gold coated (to minimize charging of sample) and then observed under a scanning electron microscope (SEM), operated at an accelerating voltage of 10 kV. Scaffold surfaces will also be studied using SEM and energy dispersive spectroscopy (EDS) to observe CaPO₄ crystals deposited by osteoblasts. The scaffold will be further analyzed using a liquid (ethanol) displacement method.

Next, mechanical properties, structural stability, and performance of implants (scaffolds) under loading will be determined by measuring the compressive strength and elastic modulus of the scaffolds. Prepared scaffolds will be tested using a universal testing machine to know the compressive strength and elastic modulus at a speed of 1.3 mm/min using ASTM D695-15 test standards. For the testing, a rectangular-shaped scaffold whose length is twice its principal width will be used. Data will be collected at 50% deformation of the initial height of scaffolds. These experiments will measure the density, percentage porosity and mechanical properties of our bioengineered bone.
2: Determine the effect of our CryoBioInk on gene expression, survival and bone differentiation potential of MSCs in microgravity. Devices will be launched and grown on board the ISS national laboratory after cryogenic storage. To study the effect of microgravity on long-term potency and the ability to form bone, we will: a. Determine the effect of our CryoBioInk in 3D scaffolds on the viability and proliferation of undifferentiated MSCs following cryogenic storage and growth in microgravity. b. Assess the effect of our CryoBioInk following cryogenic storage on osteogenic differentiation of MSCs in microgravity. c. Define the effect of CryoBioInk on osteogenic gene regulation in microgravity compared to those grown in our laboratory on Earth. The data obtained in these analyses will be an instrumental step in generating procedures for bioengineering of patient-specific off-the-shelf bone replacement devices.

MSCs show reduced potency to differentiate into bone-forming osteoblasts in microgravity and exhibit altered gene expression. However, the data obtained to date is limited to 2D cultures or cells in suspension in microgravity or simulated microgravity. Our preliminary data suggest that CryoBioInk supports a homogenous distribution of MSC in 3D scaffolds and cells can efficiently enhance osteogenesis in MSCs. To test the embedded cells in our CryoBioInk structures and assess their potential to promote bone generation in microgravity, we teamed with TechnoShot LLC as our project implementation partner.

The three-dimensional morphology adopted by cells in the absence of gravity may significantly affect cellular function. The undifferentiated MSCs will be added in CryoBioInk and will be flash frozen at −80° C., followed by securing the frozen scaffolds in a specialized bioreactor for the launch to the ISSNL. Our project will require a flight segment to the ISS National Laboratory and the return of fixed or frozen tissues back to earth. In coordination with TechnoShot, 18 ready-to use bioreactor chambers containing frozen MSC-loaded CryoBioInk scaffolds will be made by the Zalzman laboratory and will be transported to the International Space Station. The shipment will include all medium and reagents needed for 3 weeks of experiments in two segments in-orbit. Upon completion of the experiments, half of the samples will be frozen and kept in −80° C. for collection of RNA, and the rest will be fixed in 10% phosphate buffered formalin to be shipped for analyses in our laboratory.

| | | Osteogenic |
|---|---|---|
| 3D PLA Scaffold | Undifferentiated | medium |
| +CryobioInk +MSCs | n = 3 | n = 3 |
| +Cells in freezing medium | n = 3 | n = 3 |
| +Pre-attached MSCs in | n = 3 | n = 3 |
| freezing medium | | |

2a: Determine the effect of our CryoBioInk in 3D scaffolds on undifferentiated MSCs following cryogenic storage and growth in microgravity. In this sub-aim we will evaluate the potential of CryoBioInk-based medical devices in preserving cryogenic MSCs with maximal viability of MSCs after thawing. Also, we will investigate the role of such medical devices as substrates for MSC attachment and proliferation. We will assess cell viability and proliferation capacity in microgravity in undifferentiated conditions. Isogenic MSCs and three additional donors will be prepared for testing in our laboratory. Cells will be pre-stained with a long-term cell viability and proliferation tracker “Qtracker 625” which is a stable traceable label for weeks in daughter cells. Scaffolds in the bioreactor chambers will be thawed in thawing medium and will be replaced with culture media every three days for 3 weeks. To assess cell proliferation 5 micron tissue sections will be made and immunostaining of the S-G2 marker Ki67 will be performed. To assess for mitosis frequency, we will use the G2M marker phospho-H3 serine 10. Furthermore, histological sections will be used to study the distribution of MSCs in the 3D BioInk matrix to visualize cell-to-cell contact as well as extracellular deposition by the cells.

Assess the effects of CryoBioInk on undifferentiated MSC marker expression in microgravity. To define the effect of CryoBioInk on the phenotype of MSCs, scaffolds grown in undifferentiated conditions from the above described experiment in microgravity will sectioned and analyzed by immunostaining. With following undifferentiated MSC marker antibodies: anti-CD44-fluorescein isothiocyanate (FITC), anti-CD45-FITC, anti-CD105-Allophycocyanin (APC), anti-CD90-phycoerythrin (PE) and anti-CD73-PE, Anti-CD31-Alexa564. As controls, the isogenic MSCs, grown in identical conditions in our laboratory, will be analyzed. These analyses will allow us to compare the impact of microgravity on the undifferentiated/naïve phenotype of MSCs in microgravity.
2b: Assess the effect of our CryoBioInk following cryogenic storage on bone differentiation in microgravity. In this sub-aim we will test the hypothesis our CryoBioInk will protect the osteogenic potential of MSCs. Undifferentiated MSCs will be seeded in scaffolds and will be kept in cryogenic conditions at −80° C. in preparation for launch to the ISSNL. Isogenic cells from three donors will be prepared for testing in the Our laboratory. Scaffolds in the bioreactor chambers will be thawed in thawing medium and after 24 hours will be replenished with osteogenic differentiation medium. The medium will then be replaced every three days for 3 weeks. The ability to form bone will be examined by Alizarin red as a marker for bone mineralization and RNA will be taken to RT-qPCR analysis to demonstrate the expression of osteogenic markers. We will perform an RT-qPCR for activation of osteoblast markers such as COL1a1, RUNX2, Osterix/SP7, Osteocalcin (OCN), Alkaline phosphatase (ALP), and osteopontin (OPN). Data will be further validated by immunoblot. For positive controls, we will use the U2OS cell line and human bone marrow MSC lines grown in similar conditions. The data obtained in these analyses will be an instrumental step in generating the optimal protocols for generation of a medical device for patient specific bone grafting that will include MSCs.
2c: Define the effect of CryoBioInk on osteogenic gene expression in microgravity compared to those grown in our terrestrial laboratory. To uncover the effect of our CryoBioInk on osteogenic differentiation in microgravity as well as on Earth, we will perform RNA sequencing (RNA-seq) analyses from RNA samples treated as described above. All experiments will be performed in biologically independent triplicate. RNA-seq libraries will be performed according to Illumina's protocol. Library construction and sequencing will be performed at the Institute for Genome Sciences of the University of Maryland, School of Medicine (IGS).

RNA-seq data analysis: Raw sequencer data will be processed using Illumina's RTA and the Illumina cassava pipeline software for base calling and sequence quality scoring. Sequenced reads are aligned to the human genome using TopHat for sequence assessment and quality control. Gene clusters will be tested for enrichment for specific signaling pathways using pathway databases such as KEGG, Reactome and SPIKE. We have extensive experience in this technique as we previously reported.

Validation of bone gene regulation and affected pathways: We will prioritize targets identified by Next generation RNA-sequencing (RNA-seq) for validation based on (a) fold difference; (b) p-value for differential expression; (c) anticipated function based on a literature search and our preliminary data showing osteogenic and stemness genes. We will validate the effect on gene expression by quantitative real time RT-qPCR in at least 3 donors. Specific focus will be given to genes involved in epigenetic regulation, stemness potency and bone differentiation genes. Further effects on gene targets will be validated by immunostaining and immunoblot analysis as we previously described. These studies will identify the signaling pathways and mechanisms affected by microgravity and define the effect of our unique scaffolds on bone differentiation in microgravity.

Data dissemination: All data generated by this grant will be made publicly available to the scientific community in accordance the NSF guidelines. This will be accomplished through submission of data to NCBI archives and public repositories such as the GEO and SRA.

Statistical Consideration: Statistical analysis, including the assessment of study feasibility and estimation of sample sizes to meet study objectives will be done in consultation with and supervised by an expert biostatistician. All experiments will be analyzed using one-way ANOVAs. The significance will be followed by a Tukey's HSD test (STATISTICA 12). A value of p<0.05 will be considered to indicate statistical significance.

### Example 3: Tissue-On-Demand: Cryobiofabrication and Long-Term Biobanking of Live-Cell Loaded 3D Tissue-Like Structures

3D-bioprinting is a promising tool to address the continual demand of tissues and organs that are desperately needed in regenerative medicine. A combination of biomaterials and stem cells have improved our ability to generate numerous cell types, yet current culture methods allow for only limited diffusion of oxygen, CO₂, and essential nutrients. As a result, 3-dimensional (3D) organoid are limited to structures that are less than 0.5 millimeter in diameter, which do not allow for generation of human-size in vitro generated organs. Major progress has been made using 3D-bioprinting techniques to incorporate cells and stem cells into “bioinks”. However, interlayer mixing, and the need for slow printing speed, reduce cell viability and prevent creating complex-shaped multilayered tissues, which are essential for creating functional organs. As the structural stability of cell-laden bioprinted materials depends on crosslinking, which is a slow process, cell stress during and after printing is inevitable due to the extended time they must spend in non-physiological conditions. Therefore, we have developed a novel and rapid biofabrication procedure, to generate physiologically relevant sized, cells-loaded 3D structures by casting or 3D bioprinting at high-cryogenic temperatures. Our data indicate that stem cells embedded in our CryoBioink can efficiently survive after thawing and can be cryopreserved for extended periods. We further infused multipotent stem-cell (MSCs)-laden CryoBioink into 3D-printed scaffolds and show that MSCs that they efficiently survive and differentiate after storage at −80° C., proliferate and differentiated to bone like cells in 3D printed devices after thawing. Our novel approach paves the way for generating off-the-shelf tissue-engineered medical devices for regenerative medicine and may have tremendous benefits for individuals with tissue pathology, whether from disease, tumor, or trauma.

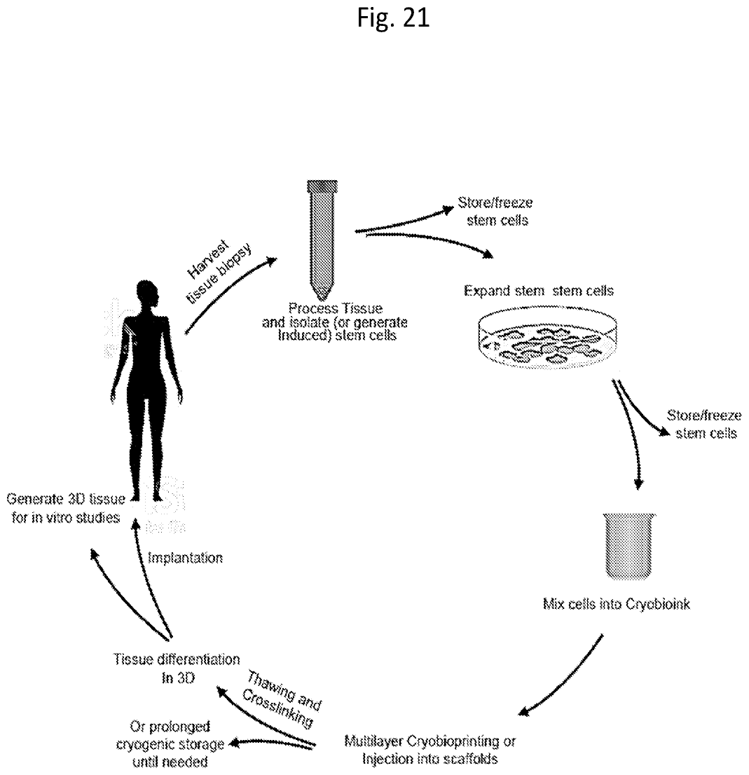

Here we describe an advanced novel approach for 3D CryoBioprinting, which enables the precise placement of layers of cells with biological growth factors in high temperature cryogenic (−80° C.) conditions, using a specialized low viscosity bioink (CryoBioink), to create tissue-mimetic complex architecture (FIG. 21). The low viscosity of the CryoBioink allows the creation of cell-loaded frozen 3D structures at low extrusion pressure which results in high cell viability. These frozen tissue-like structures can then be stored at cryogenic conditions for an extended period and thaw before application.

As a proof-of-concept, here we utilized our novel CryoBioink in high cryogenic temperature biofabrication (using casting or 3D bioprinting) technique to create 3D bone-like tissue for tissue engineering applications using an alginate-collagen-based CryoBioink, loaded with mesenchymal stem cells (MSCs). Our results show the survival, attachment, and differentiation of the MSCs in the designed scaffolds. Due to the unique composition of our CryoBioink, MSCs survive the high-temperature cryogenic fabrication and long-term storage at cryogenic condition. Moreover, MSCs efficiently differentiated into osteoblasts following thawing in osteogenic media.

Thus, our uniquely designed CryoBioink-based structure created by Cryofabrication methods can provide an off-the-shelf, ready-to-use solution in clinical applications. Furthermore, this method may be tremendously useful to create other biomimetic structures for tissue repair, reconstruction, and drug screening.

### Results

To meet the demands of tissue replacement, we first designed a novel cell-laden bioink with low viscosity and storage modulus, that allows for rapid 3D printing and prevent cell stress during printing. We further developed our bioink with cryoprotective conditions to support cell viability during printing at high cryogenic temperatures. Freezing the bioink during printing (Cryoprinting) allows for layer-by-layer fabrication of human-size multilayered-3D structures with high accuracy. During printing, each layer freezes in direct contact with the stage or with the previous layer, with minimal damage to the frozen cells during printing.

### Biofabrication of Stable, Complex Structures by 3D-Cryoprinting

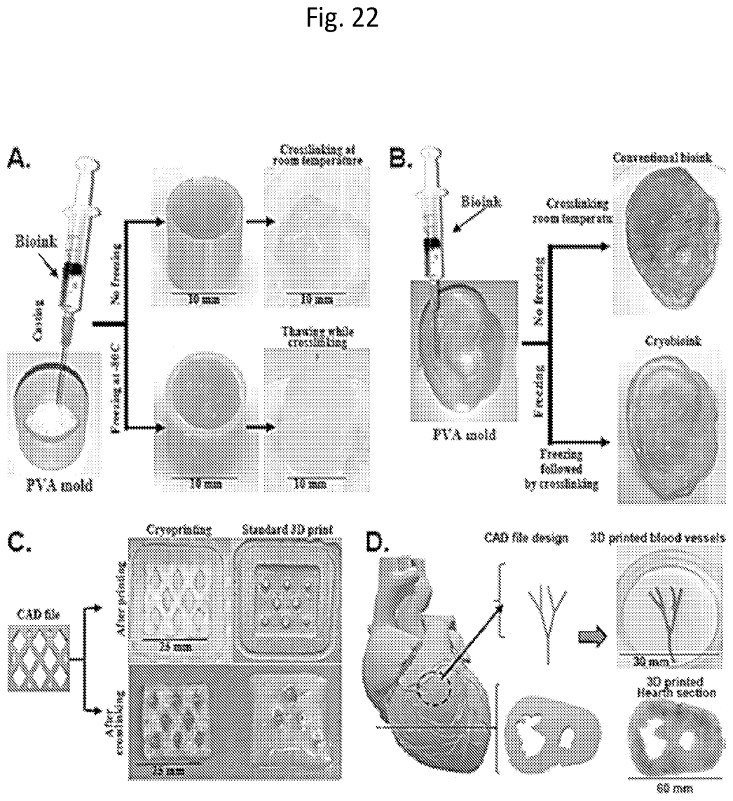

We have designed a stable, low-viscosity bioink, capable of supporting cell viability during printing and following prolonged freezing, with a base of alginate and collagen. To assess the stability of our bioink and avoid breaking the structure after casting, we 3D printed water-soluble Polyvinyl alcohol (PVA) cylinder molds. Next, our CryoBioink was casted into the PVA molds (n=6) at low cryogenic temperatures (−80° C.). Lastly, to assess the effect of freezing on the structure stability, fully frozen CryoBioink was either stored at −80° C. or taken straight to thawing in crosslinking medium containing 0.1M CaCl₂) at room temperature for 10 minutes. As controls, another set of PVA mold cylinders were casted with bioink with similar composition, without cryoprotective agents, at room temperature (22.5° C.) (n=6), and were crosslinked after casting. As expected, surface irregularities and deformed structure were consistently observed all the controls prepared at room temperature (FIG. 22A, B top panel). Conversely, fabrication at cryogenic temperature using our CryoBioink leads to a well-defined cylindrical structure (FIG. 22A, B bottom panel).

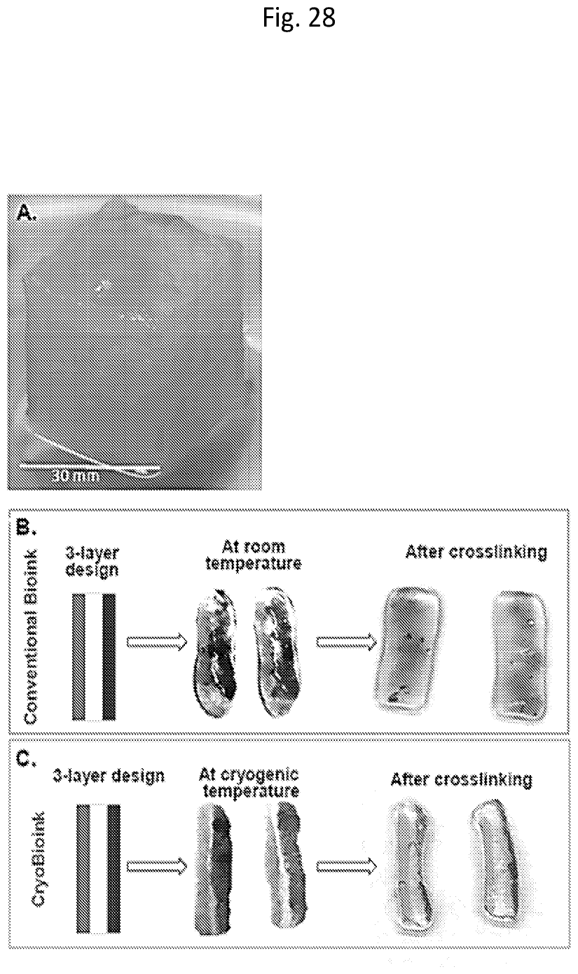

Next, to further assess our CryoBioink efficacy for generation of larger and complex structures, we 3D printed PVA molds of full-size human ears using (FIG. 22B). PVA molds (n=3 per group) were casted with control bioink at room temperatures or with CryoBioink in low cryogenic temperatures until fully frozen and then cross linked at room temperatures. As seen in FIG. 22B (Top), flat and deformed structures were generated from the control bioink, while our CryoBioink maintained solid structure with high dimensional accuracy and mechanical strength and without surface anomalies or collapse (FIG. 22B; bottom). We were able to further achieve with our method a cubical structure of a 30×30×30 cm by casting in PVA mold in cryogenic conditions followed by crosslinking in culture medium for 30 minutes (FIG. 28), indicating the robustness of our method to yield stable structures in the thickness and accuracy needed to print human size organs.

The advantage of cryofabrication using CryoBioink over the conventional 3D printing at room temperature, was further established by cryobioprinting (bioprinting at high temperature cryogenic condition at −80 C) as shown in FIG. 22 C, D. First, we used a CAD file to 3D print a 2.5×2.5 Cm square with diamond shape pattern (n=3 per group) (FIG. 22C; Left). As expected, using low viscosity Alginate: Collagen bioink for 3D bioprinting at room temperature leads to a structure collapse as is being printed (FIG. 22C; top right), and to even further structural deformation during and after crosslinking (FIG. 22C; bottom right), and the resultant 3D shape was flatter, arger and no longer similar to the 3D model. Conversely, using our CryoBioink printed at cryogenic conditions, leads to the correct size and thickness expected from the CAD design with a well-defined porosity and structure.

Next, to further validate the efficacy of the designed bioink in creating tissue-like, more complex structures using cryobioprinting method, we created a vascular network (1 mm thick and 25 mm long) (FIG. 22D; top). Then we sought to demonstrate the ability of our cryprinting approach to print large, complex anatomical architectures. As a proof of concept, we have cryoprinted a section of a human heart at 10 mm/s print rate using a CAD file, based on a digital design (FIG. 22D; bottom). Following freezing at −80 C and crosslinking for 10 minutes, a structural complexity was achieved using our cryobioprintring method, indicating a detailed anatomical structure, with mechanical stability and robustness can be achieved using 3D printing at cryogenic conditions, allowing rapid and increased accuracy.

### Cryobioprinting of Cell-Laden Bioink Allows for Generation of Multilayers 3D-Structures

One of the major challenges in designing 3D printed organs, is the ability to accurately place many different cell types in multilayered, complex anatomical architectures. To further test our CryoBioink in creating multilayers and assess for interlayer mixing, we prepared CryoBioink (without cells) and then divided it to three parts to dye it either with a red color (add dye name), blue or left it transparent. Then, using a syringe we hand printed three-layered structure in cryogenic conditions. Our results indicate that the 3 layers quickly diffuse at room temperature, and further diffuse during crosslinking, leading to a resultant structure that is deformed, and the layer no longer show clear separation (FIG. 28B). Yet, as a result of printing at high cryogenic temperatures (−80 C), each layer freezes during printing, which allows the layers to be placed side by side without interlayer mixing (FIG. 28C), and therefore, 3 defined layers are formed following thawing in crosslinking solution. This indicate, that cryoprinting has superior efficiency in printing multilayered structures, compared to printing at room temperature.

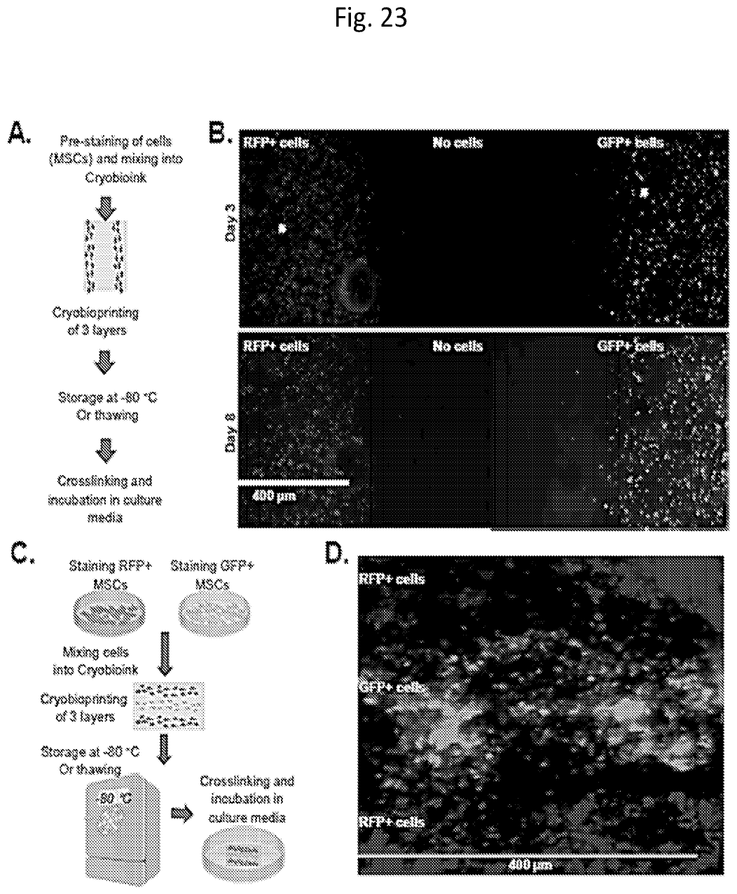

Then, to evaluate cell-laden CryoBioink for cell interlayer mixing at the microscopic level, we prepared our CryoBioink with either cells (Tu167) expressing red fluorescence protein (RFP+), or with green fluorescence protein (GFP+), while a third CryoBioink with the same composition remained without cells. We then printed side-by-side the RFP+ cells, an a-cellular layer and a GFP+ cells layer in cryogenic conditions (FIG. 23A). Our results indicate that our novel bioink can be used to create complex, well-defined multilayered, multicellular 3D architecture of the incorporated cells based on CAD file. Moreover, no mixing of layers at the interface was observed (FIG. 23B).

To further assess our CryoBioink in creating a multilayer 3D structure from stem cells, we next prestained multipotent stromal stem cells (MSCs) with either red fluorescence (Cell Tracker Red CMTPX) dye, or green fluorescence dye (Green CMFDA). We then used our cell-laden CryoBioink to create a 3 layered 3D structures in cryogenic conditions (FIG. 23C). Cross sections of the 3D structure demonstrate the 3 distinct layeres with high accuracy and without interlayer mixing (FIG. 23D). Therefore, this procedure opens the opportunity to create complex tissue-mimicking structures.

### CryoBioink Printed Cells Maintain High Viability in Prolonged Culture and Storage

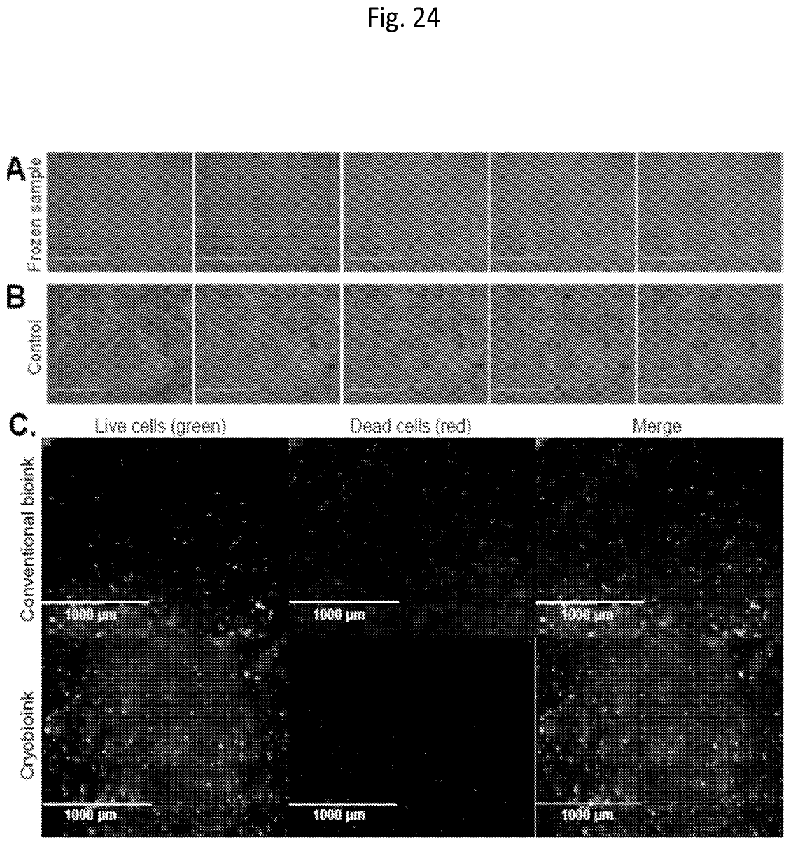

After validating the designed CryoBioink in creating well-defined 3D structures in cryogenic condition, we examined the effect of cryopreservation in our Cryobioink on multipotent stem cell. Unstained MSCs were mixed into our CryoBioInk, casted into PVA molds, and then flash frozen and stored at −80° C. for one week. For cell viability evaluation, the frozen scaffolds were then immersed in culture media at 37° C. containing crosslinker (CaCl₂)), washed and incubated in media in a at 37° C., 5% CO₂ humidified incubator. The PVA mold was then dissolved, and the media was changed after 2 hours and again after 24 hours to remove any PVA left. For cell maintenance, the medium was replaced every three days. For the control samples, scaffolds were prepared at room temperature, crosslinked, and directly placed in culture media after the crosslinking, and henceforth treated the same as above. After 8 days in culture, viable cells were visualized under a fluorescence microscope using the fluorescence Cell Tracker Red CMTPX dye which allows us to visualize only viable cells. Our data show a homogenous distribution of viable MSCs throughout the CryoBioInk matrix (FIG. 24A). Images were captured at different depths and their vertical stacking confirmed the presence of cells and their homogenous distribution in three-dimensional (3D) space. Interestingly, the red fluorescence as well as the cell morphology in the CryoBioink and the cell number was superior to that of the control samples and confirmed high cell viability, attachment and proliferation in the designed CryoBioInk scaffold, while the controls cells indicated 50% viable cells survived throughout the layers (FIG. 24B). Our data indicate that CryoBioInk preserves the viability of the cells in 3 dimensions in cryogenic storage.

### Designing of CryoBioink of Low Viscosity and Storage Modulus

As the shear stress created during the extrusion process can cause cell death, therefore, the rheological properties of bioink were studied using a rheometer. The amplitude sweep at constant frequency showed a significantly low value of storage and loss modulus (FIG. 8). The amplitude sweep provides information about the viscoelastic behavior of bioink, the value to strain at which bioink change from a more solid-like phase to more liquid-like phase.

Although high viscous bioink with high storage modulus is not required in the current method of bioprinting (bioprinting at high temperature cryogenic), it is important to know about the flow initiation and yield stress which give an insight about the printability of the designed bioink. A shear stress ramp is used to determine the yield stress of bioink. The plot between shear stress and viscosity provides the value of stress at which bioink starts flowing during extrusion (FIG. 8). We observed a decrease in the viscosity of bioink with the increase in the shear stress. At ˜1 Pa shear stress, the viscosity of bioink was 10 Pa·s, which decreases linearly until ˜5 Pa, and after that, a steep decrease in the viscosity was noted due to the yielding of bioink, which confirmed the printability of the designed bioink. Our bioink demonstrated a significantly lower value of yield stress (˜5 Pa) than the existing bioinks (70-200 Pa) (Paxton et al., Biofabrication, (2017), 9:044107). Furthermore, our bioink also showed a shear-thinning behavior, characterized by a decrease in viscosity with an increase in the shear rate (FIG. 8).

### CryoBioink Composition and Microstructural Analysis

Since the composition and porosity (including pore size and surface topography) of the scaffolds can significantly affect the cell-materials interaction and, eventually, the biocompatibility, therefore, compositional and microstructural analysis of the scaffolds was carried out using FT-IR and cryo-SEM, respectively.

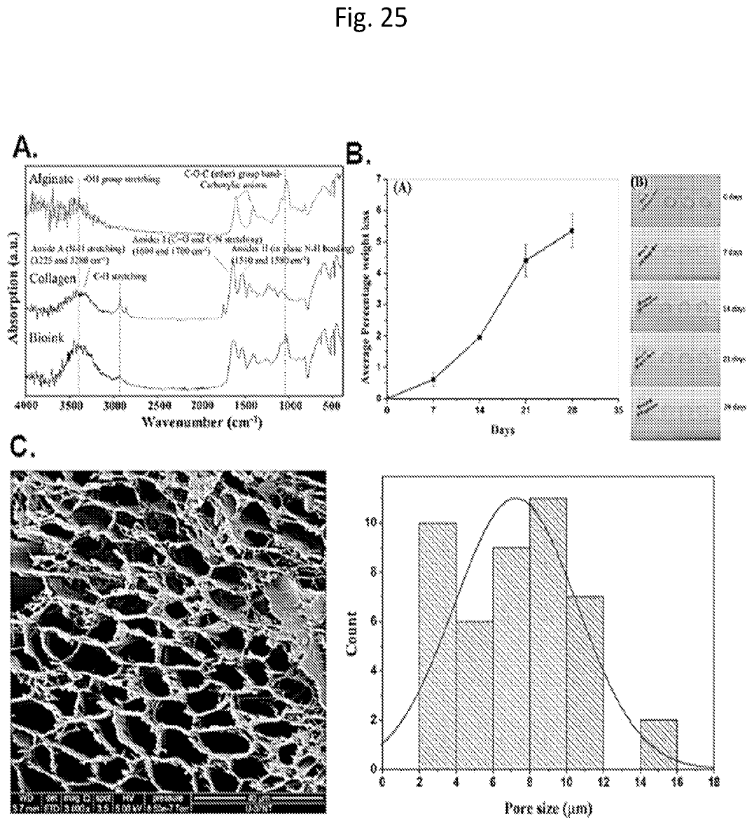

The results of FT-IR analysis of the 50Alg-50Col bioink were compared with pure alginate and collagen powder (FIG. 25). The absorption peaks at 3450 cm⁻¹, 1618 cm⁻¹, 1440 cm⁻¹, and 1050 cm⁻¹ corresponding to—OH group, —COO—, —COO—, and C—O, respectively, confirmed the presence of alginate. The presence of absorption peaks corresponding to Amide A (N—H stretching, 3225 cm⁻¹, and 3280 cm⁻¹), Amide I (C═O and C—N stretching, 1600 cm⁻¹ and 1700 cm⁻¹), and Amides II (in-plane N—H bending, 1510 cm⁻¹ and 1580 cm⁻¹) confirmed the presence of collagen. The intensity ratio of absorption peaks at 1235 cm⁻¹ and 1450 cm⁻¹ close to 1 is an indicator of the integrity of the triple-helical structure of collagen. We found this ratio equals 1.015 and 1.146 in collagen prepared by the pepsin digestion at acidic pH and collagen in the prepared bioink, respectively.

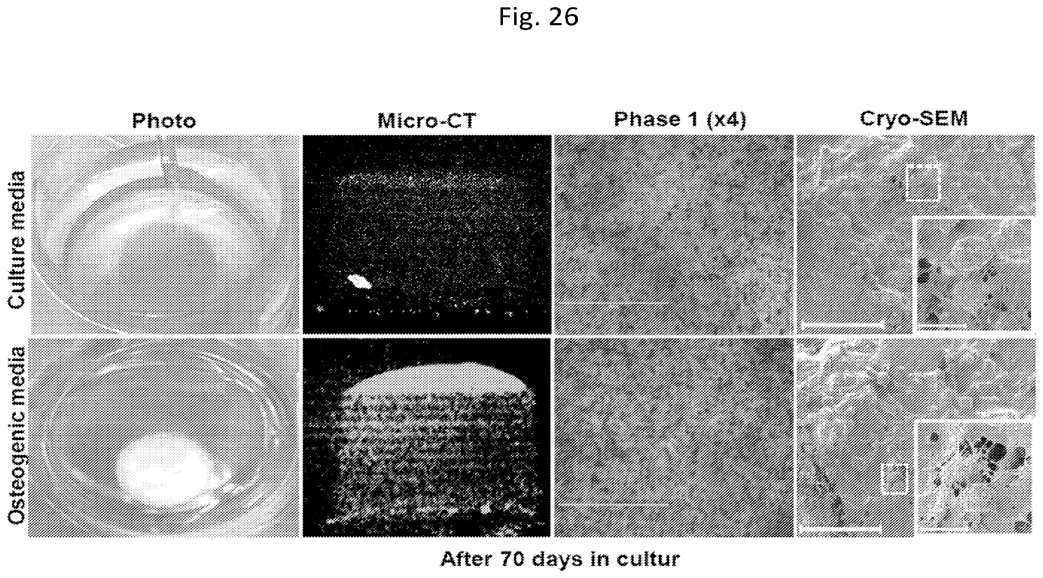

The microstructural analysis using cryo-SEM confirmed the porous structure of CryoBioink scaffold with pore size distribution from 2-16 μm (FIG. 26). The mean pore size was 6-8 μm.

### Mechanical Properties of the Cryobioprinted Structures

Compression testing of CryoBioink scaffolds at 37° C. in submersion condition (in 1×PBS with Ca and Mg) showed a compressive strength of 2.520±0.235 kPa. The stress-strain curve showed multiple yield points and an increase in the stress value after fracture point (FIG. 25A). The calculated value of yield strength and bulk elastic modulus of scaffolds were 1.720±0.143 kPa and 0.069±0.015 kPa, respectively.

We next assessed the dissolution behavior of the cryobioprinted structures. Our results of 28 days dissolution study showed a time-dependent loss in weight of scaffolds, which confirms the biodegradability of the designed bioink (FIG. 25B). Furthermore, no change in pH was noted during the course of the study. Also, no dimensional change in the scaffolds due to swelling was observed, which confirms the structural stability of the scaffolds (FIG. 25B).

### Cryobioprinted Structure to Promote Osteogenic Differentiation of Stem Cells

The light microscopy of the scaffolds revealed the tissue-like structure with high density of cell in three-dimensional space in both culture media and osteogenic differentiation media (FIG. 26).

After 70 days of culture, CryoSEM revealed the presence of cells attached to the bioink matrix without any sign of dead cells (FIG. 26). The higher magnification images confirmed the typical osteoblasts morphology with spreading of filipodia and integration with substrate. Moreover, the cells in osteogenic media appears smaller than the cells in culture media suggesting differentiation to osteoblasts. (FIG. 26).

Furthermore, the microCT confirmed the presence of Ca—PO₄ crystals (white in color) in scaffolds kept in both cell culture media and osteogenic differentiation media (FIG. 26). However, as compared to culture media, the density of Ca—PO₄ was higher in osteogenic media. Importantly, deposition of the bone mineral was higher in circumference region than the center.

Our H&E staining of cryosections shows the tissue-like structure with homogeneous distribution of cells (cytoplasm is stained in pink and nucleus in purple) throughout the scaffolds. We noted higher number of cells with smaller size in osteogenic media compared to cells in culture media. Previous study has confirmed smaller size of osteoblasts compared to undifferentiated cells [Ref: https://doi.org/10.1002/jbm.a.35567].

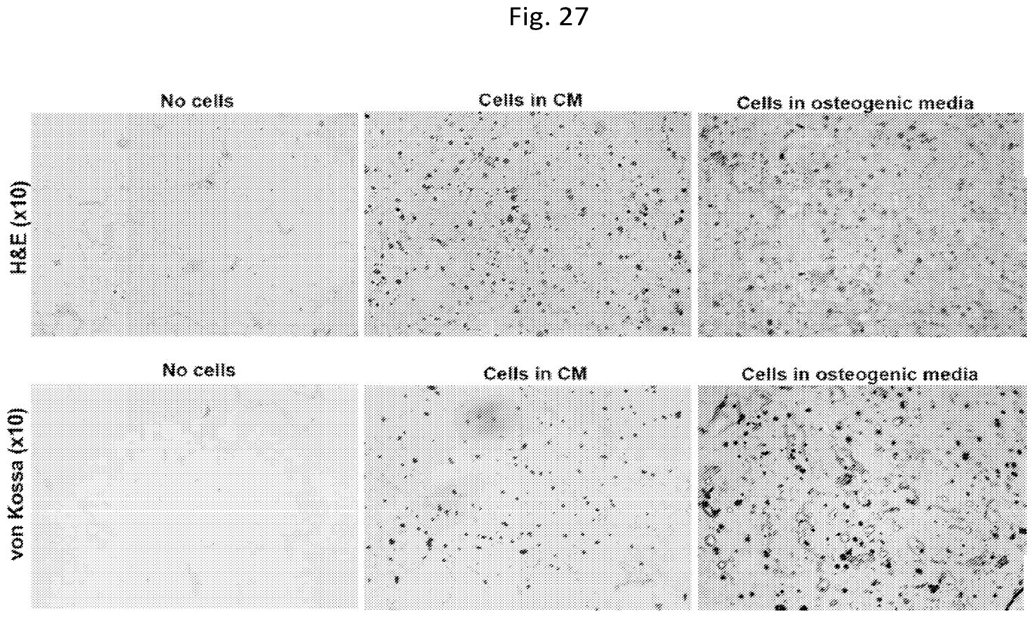

The von Kossa staining of samples confirmed the mineralization (due to osteoblasts activity) of scaffolds kept in both culture media and osteogenic differentiation media, confirms the osteogenic differentiation of MSCs (FIG. 27). However, the volume of the deposited mineral was higher in the case of osteogenic media than culture media (FIG. 27).

### Discussion

Various bioinks have been designed to create self-standing structures either by bioprinting or templet casting methods. In this list, alginate based bioinks are found very attractive due to their ability to effortless chemical crosslinking using divalent metal ions, such as Ca²⁺, Fe²⁺, etc. to make a mechanical stable 3D structure.

Designing of tissue analog 3D structures requires optimization of many interrelated parameters of bioinks to ensure survival and functionality of cells during bioprinting. For instance, bioink properties, such as viscosity and storage modulus directly affect the shape fidelity of the printed structure. The high shape fidelity is define by the shape retention ability of the bioink, depends on the storage modulus and loss modulus (components of the dynamic modulus) of a bioink. A higher storage modulus (G′) represents more solid-like property, while a higher loss modulus (G″) means more liquid-like property. The storage modulus is simply a measure of the energy required to distort a hydrogel (bioink) structure. This means higher storage modulus improves the mechanical strength of the printed structure by enhancing the ability of the hydrogel to store deformation energy and recover in an elastic manner. The higher storage modulus can be achieved by increasing the viscosity of bioink. Therefore, high viscosity bioinks are generally used to create structurally stable construct for tissue engineering purposes. However, a high extrusion pressure is applied to extrude such high viscosity bioink. A high extrusion pressure leads to the generation of high shear stress on the cells, costing the viability of cells. Also, such parameters result in slow bioprinting, which force cells to stay in non-physiological environment for the longer period, which can further affect the quality of the created structures. Moreover, the requirement of crosslinking of individual layer in bioprinting further increases the printing time. Apart from these, difficult to create complex structures due to interlayer mixing and incapacity to make larger 3D structures than few millimeters, restrict the designing of clinically useful tissue structures. Therefore, to address these challenges, we developed a novel alginate and collagen-based low viscosity cell-loaded CryoBioink. The designed alginate-collagen (Alg-Col) CryoBioink characterized by low viscosity (˜10 Pa·s) and storage modulus (1.5×10⁻⁵ mPa). A low viscosity bioink with a lower yield point and shear thinning behavior minimizes the possibility of shear stress-induced cell death during bioprinting. Also, it allows rapid bioprinting (high speed printing) due to flow initiation at low shear stress.

The decrease in viscosity and thus reduction in storage modulus leads to poor shape fidelity. Therefore, to address this challenge, we further developed a new bioprinting method, involves bioprinting at high temperature cryogenic condition. As we know that one of the major challenges in designing 3D printed organs is the accurate spatial placement of many cell types in a complex multilayer structure. To this end, interlayer mixing is difficult to prevent in layers printed adjacent to each other. In this newly developed method, during fabrication of 3D structure, each layer freezes instantaneously in direct contact with the undercooled stage or previously deposited layer without mixing layers at the interface. The printing at such a low temperature can cause significant cell death. Therefore, to solve this problem, we added a cryoprotectant to the bioink to prevent cell death due to freezing.

The frozen structures of cell-loaded bioink with cryoprotectant in it can be stored at −80° C. or in liquid nitrogen for a longer period. Apart from ease in printing complex structures, the freezing condition led us in designing of mechanical stable structure with dimensional accuracy after thawing in calcium chloride (crosslinker)-based culture media. It is important to note that in conventional methods of biofabrication it is difficult to achieve dimensional accuracy and structural complexity. Additionally, requirement of crosslinking of each layer and slow bioprinting in conventional method leads to cell death.

Alginate is used here as a base material in the bioink, facilitate easy extrusion of CryoBioink through a narrow needle and also enables efficient the crosslinking with calcium chloride (CaCl₂)). Alginate shows strong chelation in the presence of divalent cations such as Ca²⁺ and thus provides a feasible way for faster crosslinking at physiological conditions without causing cell death. Alginate is an anionic polysaccharide, and a linear copolymer of β-(1-4)-linked D-mannuronic acid and β-(1-4)-linked L-guluronic acids. Although alginate is a biocompatible material, it is bioinert in nature and thus does not promote cell adhesion. Therefore, to enhance cell adhesion we assessed a combination of alginate-collagen. Collagen in the CryoBioink acts as a biocompatible extracellular matrix-like substrate for the cell attachment and proliferation.

The physical properties of bioink was enhanced by initiating crosslinking of collagen to alginate, mediated by carbodiimide (EDC/NHS). The EDC was used as a primary crosslinker, which links the carboxyl-acid group of alginate, with the primary amines of collagen. Since EDC-mediated crosslinking is only effective at acidic pH, NHS was used to improve the efficiency of crosslinking at pH 7.4. After scaffolds casting or printing, to enhance structural stability, CaCl₂) was used as a secondary crosslinker, to facilitates linkage of a-L-guluronic acid (G-blocks) of one alginate chain with the G-blocks of adjacent chain through Ca²⁺. Important to mention that the use of EDC/NHS may reduce the cell binding sites on collagen. The cell proliferation and differentiation of stem cells in our scaffolds can be associated with extracellular matrix deposited by cells during the long-term culture, which provides an ideal substrate for cell attachment and migration. Therefore, for the cryobioprinting, we also designed a CryoBioink without EDC/NHS by simply mixing alginate and collagen in a cryoprotectant (10% DMSO and 90% FBS) at 4° C.

The melting of ice crystals in the frozen CryoBioink samples create interconnected pore channels with size (6-8 μm) in the range of blood capillaries, are expected to help in efficient transportation of oxygen and nutrient to the cells growing inside the scaffold. Therefore, CryoBioink and cryobiofabrication method found advantageous over the conventional bioinks and biofabrication in the designing of clinically relevant sized big scaffolds. Fluorescence microscopy of big scaffolds confirmed the presence of live cells in the center of scaffold, which emphasize the benefits of pores channels in cryobiofabricated scaffolds. Although it is not in the scope of this study, a controlled cooling and cryoprotectant percentage can be a useful tool to create different pore shape and size. Also, directional colling can be used to create aligned pores in the cryofabricated scaffolds. The aligned pores can be used to create muscle-like structure or for the directional growth of nerve.

Structural resilience is required for the safe handling of scaffolds and for the implantation. In this context, the compression testing of cylindrical scaffolds of CryoBioink at 37° C. in in 1×PBS (with Ca and Mg) showed a compressive strength of ˜2.520 kPa which increased before fracture. This increase in the stress value after fracture is a result of densification can be related to the closure of pores at higher compression. The bulk elastic modulus of scaffold was in the range of soft tissue with a value of ˜0.069 kPa. The value of yield strength (yield point) was also very low, ˜1.720±0.143 kPa, which signifies the small elastic region and thus a narrow range in which scaffolds can be compressed. At the yield point, the linear (elastic) region of the graph terminates, and after this, the material behaves plastically, and elastic recovery is not possible after this point.

Important to mention that the composition of the bioink in this study has been designed to only show the efficacy of this novel cryobiofabrication method by freezing the to create a high fidelity structure using a low viscosity CryoBioink without causing cell death and interlayer mixing. However, depending upon applications, the strength of scaffolds can easily be tailored by changing the composition of the CryoBioink as well as crosslinking parameters. The ease of alteration in the mechanical and physical properties makes the designed bioink suitable for various tissue engineering applications ranging from skin, heart, lung, kidney, liver, cartilage etc.

The designed bioink can be used for loading and delivery of drugs and growth factors at the defect site for the faster regeneration of damaged tissues. In this context, dissolution behavior is a critical parameter required for the controlled delivery of drugs of growth factors. FIG. 25B showed a steady dissolution profile with no abrupt increase in the dissolution of scaffolds during the course of 28 days dissolution study. Such a controlled dissolution of the scaffolds prevents the burst release of drugs and thus can prevent the pharmacologically dangerous effects (Costa et al., European journal of pharmaceutical sciences, (2001), 13:123-133). We observed no noticeable change in the dimension of scaffolds during the dissolution period, while a weight loss was noted. This confirms the volumetric weight loss during the dissolution study.

Although the designed bioink and fabrication method are suitable in creating any tissue types (heart, lung, kidney, liver, cartilage etc.), in the current project, for the proof of principle, we, studied the osteogenic differentiation of stem cells in the bioink. Considering bone as mechanically strong tissue and bioinks are mechanically weak, we created a hybrid structure of 3D printed PLA scaffold with varying porosity, infilled with CryoBioink, followed by freezing at −80° C. and then crosslinking with 0.05 M CaCl₂). In the hybrid structure, PLA scaffold provides the mechanical stability while alginate-collagen-based our CryoBioink provides an ideal environment for the bone forming cells for faster bone growth (Kumar et al., Journal of Tissue Engineering and Regenerative Medicine, (2018), 12:1133-1144.). Important to mention that the PLA scaffolds can be replaced with high strength titanium or titanium alloy scaffolds. Using this method, we further created a human mandibular defect size stem cells-loaded hybrid scaffold.

Since the focus of current work is the CryoBioink and it is the main bioactive component of the designed hybrid structure, we studied the viability and osteogenic differentiation of stem cells. For the osteogenic differentiation study, human stem cells-loaded cylindrical scaffolds of CryoBioink were incubated in the osteogenic differentiation media for 70 days. Micro-CT showed the deposition CaPO₄ mineral in the scaffold. The micro-CT data was supported by the von-Kossa staining calcium nodules deposited by osteoblasts cells. This confirms survival of stem cells in long-term culture condition and differentiation into osteogenic lineage, which is further confirmed by the Cryo-SEM. The Cryo-SEM clearly showed the presence of osteoblasts-like cells in the bioink matrix. Immunostaining and H&E staining of samples shows the high cell viability and homogenous cell distribution with tissue-like structure.

### Conclusion

We have developed a novel low viscosity (a low storage modulus) CryoBioink for the 3D bioprinting at high temperature cryogenic condition without causing shear-induced cell death. This is the first work which reports the 3D biofabrication (bioprinting and casting) of high-fidelity multilayered structures for various tissue engineering applications without the need of instant crosslinking and high viscosity bioink. The created cell-loaded 3D structures can be stored at −80° C. or lower temperature and can be revived easily later time without losing cell functionality and structural integrity.

The new CryoBioink and Cryofabrication present a major advancement in creating of tissue-mimicking complex structures with spatial resolution and without the mixing of adjacent layers in a multilayered structure. The future development of this technology may allow the generation of off-the-shelf clinical size living grafts for reconstructive surgeries.

### Methods

### Synthesis of Novel CryoBioink for 3D Cryobiofabrication

For the bioink, first stock solution of 2 wt. % alginate was prepared by dissolving 2 g of alginate in 10 ml sterile deionized (DI) water at room temperature for stirring at 300 rpm for overnight. To make 3 wt. % collagen, first 1% collagen was prepared and then volume was reduced to one third by centrifuging it for 10 min at 4500 rpm. To make 1% collagen, first 15 mg pepsin was dissolved in 15 ml 10 mM HCl at room temperature, followed by adding 150 mg collagen and kept on magnetic stirrer for 48 h for the collagen gel formation. After the gel formation, the pH was adjusted to 7.4 using 10M NaOH. The prepared alginate and collagen gels were stored at 4° C. until use.

For 10 ml alginate-collagen (Alg-Col) CryoBioink preparation, first 2.5 ml 2% alginate was mixed in 2.5 ml freezing media (FBS with 10% DMSO) to make 1% alginate and stored at 4° C. Also, 2.5 ml 3% collagen was mixed in 2.5 ml freezing media to make 1.5% collagen and stored at 4° C. Next, 12.5 mg EDC was mixed in 5 ml 1 wt. % alginate for 15 min at room temperature and slow stirring condition, followed by adding 7.5 mg NHS and mixing for another 15 min. Then, 5 ml 1.5% collagen was added in this and mixed for 1-2 min to make Alg-Col CryoBioink. Next, cells were added in this bioink and centrifuged at 500 rpm for 1 min to remove trapped air from the CryoBioink. This cells-loaded CryoBioink was filled in a sterile 10 ml syringe and used for creating 3D scaffolds either by casting in a PVA mold or by 3D cryobioprinting. The designed scaffolds were stored at −80° C. until use. This CryoBioink with EDC/NHS was also used for the rheological study and in the designing of scaffolds by casting.

To enhance the cell attachment, we also created a CryoBioink without EDC/NHS. For this, first 30 ml 1% collagen was centrifuge at 4500 rpm for 10 min. After discarding supernatant, collagen was resuspended in the freezing media to a final volume of 10 ml. Next, 0.2 g alginate was added to this solution and kept for stirring at 300 rpm at 4° C. for the overnight.

It is important to mention that the focus of this work is to design a fabrication method and bioink to create complex structures using low viscosity bioink in cryogenic condition to maintain high cell viability and shape fidelity. This work provides flexible method for the designing of CryoBioink depending on applications. Therefore, different CryoBioinks can be created by mixing cryoprotectant with biopolymers in an optimal ratio.

### Analysis of the Phase in the Designed Bioink

Fourier transform-infrared spectroscopy (FT-IR, model: Nicolet 6700, Thermo Fisher Scientific, USA) was used for the study of bioink composition by determining functional groups of alginate and collagen. For this, freeze-dried bioink samples were used to record the FT-IR data in ATR mode from 4000 to 400 cm⁻¹. The obtained data was compared with pure alginate and collagen.

### Investigation of Microstructure of the 3D Scaffold

A cryo-scanning electron microscope (cryo-SEM, model: Quanta 200, FEI, USA), equipped with cryo-transfer unit (model: ALTO2100, Gatan, USA) was used for the microstructural analysis in the cryofabricated samples. To determine the microstructure, designed scaffolds without cells were fixed for 16 h in 10% formaldehyde at 4° C. Thereafter, a thin section of the scaffolds was cut and adhered to an aluminum stub using a cryoembedding solution, followed by freezing in slush nitrogen (created from liquid nitrogen to avoid Leidenfrost effect). Prior to observation, the surface of frozen samples was fractured and sublimated to expose the inner microstructure. Thereafter, samples were gold coated (required to minimize the image distortion caused by the charging effect) and observed under a cryo-scanning electron microscope in secondary electron mode, operated at an accelerating voltage of 5 kV and a working distance of 6 mm. A low temperature of −130° C. was maintained inside the SEM throughout the imaging process.

### Rheological Properties of Designed Bioink

A dynamic stress rheometer (model: HR30, TA Discovery, USA) with a cone and plate geometry (Cone angle: 1 degree and truncation gap: 26 μM,) was used to measure the storage and loss modulus at room temperature. After cleaning of plate with DI water, 300 μL bioink was added on the plate for the testing. First, amplitude sweep (Oscillation strain sweep: 10⁻² to 10²) at 1 Hz frequency was carried out to determine the storage modulus and loss modulus values. These data were used to find the viscoelastic properties of bioink and recovery of bioink after the transition from a more solid-like phase to a more liquid-like phase during extrusion. Furthermore, shear rate vs. shear stress and shear stress vs. viscosity were measured to determine the properties of bioink under the effect of shear stress.

### Fabrication of Three-Dimensional (3D) Structures

3D structures were created either by 3D bioprinting or by casting of bioink in water-soluble polyvinyl alcohol (PVA) mold or under high temperature cryogenic condition, maintained by dry ice (−78.5° C.). An inhouse developed bioprinter with positive displacement-based syringe extruder was used for the 3D bioprinting using computer aided design (CAD). A ½″ blunt stainless-steel needle of 25 G (Inner diameter of 250 μm) was used for cryobioprinting. To optimize bioprinting parameters, CryoBioink (without cells) was extruded manually in a well plate, kept on dry ice to create multilayered layers structures. Bioprinting in this condition allows immediate freezing of printed layers and thus providing adequate mechanical strength for successive layers in the printed structures. To further validate the efficacy of our novel bioink in creating scaffolds using an automated fabrication system, 3D mesh structure with diamond shaped pores was created at a printing speed of 10 mm/s in high temperature cryogenic condition. Additionally, tissue-mimetic human heart slice was bioprinted in cryogenic condition. The printed frozen scaffolds were stored at −80° C. After 24 h, frozen scaffolds were thawed for 10 min in a prewarmed (at 37° C.) culture media supplemented with the crosslinker (0.05 M CaCl₂)). To compare, similar structures were created at ˜37° C. using same bioink, followed by crosslinking for 10 min in prewarmed (at 37° C.) 0.05 M CaCl₂).

For casting, we created 3D printed molds of water-soluble polyvinyl alcohol (PVA), followed by injecting CryoBioink into these molds (n=6). PVA molds were created using a fused deposition modeling (FDM) printer (S5, Ultimaker) with 0.4 mm heated nozzle. To assess the beneficial effect of freezing in creating well defined structure as compared to conventional methods (where freezing is not an integral part of biofabrication), PVA molds with CryoBioink were stored at −80° C. for freezing (for 24 h) prior to crosslinking by thawing these frozen scaffolds in culture medium containing 0.05 M CaCl₂) at room temperature for 10 minutes. For the comparison, we created 3D scaffolds by conventional method, which involves casting of bioink (without cryoprotectant) in a PVA mold at room temperature (22.5° C.) (n=6), followed by direct crosslinking in culture medium containing 0.05 M CaCl₂) at room temperature for 10 minutes. No freezing involves in this method.

To further assess our CryoBioink efficacy for generating larger clinically relevant complex structures, we 3D printed PVA molds of full-size human ears using FDM printer. PVA molds (n=3 per group) were casted with control bioink (without cryoprotectant) at room temperatures or with CryoBioink in high cryogenic temperatures and then cross linked at room temperatures.

To further test our CryoBioink in creating multilayers and assess for interlayer mixing, we prepared CryoBioink (without cells) and then divided it to three parts to dye it either with a red color (alizarin red), blue (hematoxylin) or left it transparent (without dye). Then, using a syringe we hand printed three-layered structure at room temperature.

Then, to evaluate cell-laden CryoBioink for cell interlayer mixing at the microscopic level, we prepared our CryoBioink with either cells (Tu167) expressing red fluorescence protein (RFP+, Cell Tracker Red CMTPX), or with green fluorescence protein (GFP+, Cell Tracker Green CMFDA), while a third CryoBioink with the same composition remained without cells. We then printed side-by-side the RFP+ cells, an acellular layer and a GFP+ cells layer in cryogenic conditions. To further assess our CryoBioink in creating a multilayer 3D structure from stem cells, we next prestained multipotent stromal stem cells (MSCs) with either red fluorescence (Cell Tracker Red CMTPX) dye, or green fluorescence dye (Cell Tracker Green CMFDA). We then used our cell-laden CryoBioink to create a 3 layered 3D structures in cryogenic conditions.

### Effect of CaCl₂) Concentration and Crosslinking Time on Cell Viability

The optimal concentration of CaCl₂) was determined by incubating MSCs (2.5×10⁴ cells/ml) in culture media supplemented with different concentration of CaCl₂) (0.05M, 0.1M, 0.2M, 0.5M, 0.8M, and 1M) for various duration (10 min, 30 min, 1 h, and 5 h), followed by LIVE/DEAD™ cell staining (Invitrogen), marking live in green and dead cells in red.

Thawing of Cells-Loaded 3D Scaffolds after Storage −80° C.

After validating the designed CryoBioink in creating well-defined 3D structures in high temperature cryogenic condition, we examined the effect of cryopreservation in our CryoBioink on multipotent stem cell. Prestained MSCs with Cell Tracker Red CMTPX dye were mixed into our CryoBioink, casted into PVA molds, and then flash frozen and stored at −80° C. for one week. For cell tracking and distribution, the frozen scaffolds were then immersed in culture media at 37° C. containing crosslinker (0.05M CaCl₂)) for 10 min, followed by washing with fresh culture media. Then the samples were incubated in the culture media at 37° C. and 5% CO₂ humidity. For complete removal of the dissolved PVA mold, media was changed after 2 hours and again after 24 hours. For cell maintenance, the medium was replaced every three days. For the control samples, scaffolds were prepared at room temperature and directly placed in the culture media after the crosslinking, and henceforth treated the same as above. After 8 days in culture, proliferating cells (viable cells) were visualized under a fluorescence microscope with the help of inherited cell-tracker dye by the live cells.

To see the protective effect of cryoprotectant (DMSO) on cells (MSCs, D11P17-Z4)-loaded in the bioink exposed to the freezing temperature, 3D structures were created using bioinks with and without DMSO. For this, structures were designed by hand printing in an ultra-low attachment 6-well plate, kept on dry ice before and during printing. The designed frozen scaffolds were stored at −80° C. To study the cell viability, after 24 h, frozen scaffolds were thawed for 10 min in a culture media containing 0.05 M CaCl₂) (prewarmed at 37° C.). After 2 days, a thin section of cells-loaded structure was stained with LIVE/DEAD™ cell imaging kit to quantify cell viability.

### Mechanical Properties

To measure the compressive strength of the cryofabricated structures, we designed cylindrical scaffolds of 9 mm diameter and 6 mm height. We followed inequality criterion for maximum nominal compressive strain to determine the adequate dimension for the compression testing of hydrogel scaffolds, as per ISO 604:2002 standard:

$${ε}_{c} \leq 0 \cdot 4 \frac{{diameter}^{2}}{{length}^{2}}$$

Note that the theoretical strain values lie between 0 to 1. Or in percentage 0 to 100%.

Scaffolds for the testing were fabricated by casting in a PVA mold, followed by storage at −80° C. Next day, scaffolds were thawed in 0.05M CaCl₂), prepared in DI waster followed by washing twice with 1×PBS (with Ca, Mg). Furthermore, 1×PBS was changed after 30 min, 2 h and 24 to completely remove the PVA mold. At least five samples were tested to obtain a statistically relevant data.

A dynamic mechanical analyzer (DMA, model: DMA850, TA Discovery, USA) was used to measure the compression properties at 37° C. in submersion condition in 1×PBS (with Ca, Mg). Testing was carried out at rate control-strain ramp mode at a strain rate of 1%/min and 0 kN preload.

### Stability of the Designed Scaffolds in Aqueous Media

To determine the stability and integrity of the designed scaffolds, a dissolution study (with Ca, Mg) was conducted for 7, 14, 21, and 28 days in 1×PBS. The crosslinked (with 0.05M CaCl₂)) cylindrical-shaped scaffolds (of dimension 9 mm×6 mm) were washed with 1×PBS to ensure the removal of PVA mold and excess CaCl₂). At least three samples were used in each category. After taking initial weight measurements, scaffolds were kept in a CO₂ incubator during the entire duration of the dissolution study. After 7, 14, 21, and 28 days of incubation, the solution from each well was carefully removed, and the final weight was measured.

Furthermore, swelling behavior of scaffolds was studied in 1×PBS (with Ca, Mg) at 37° C. The result of this study is presented as a supplementary data. For this, crosslinked samples (with 0.05M CaCl₂)) were freeze-dried, and weight was measured (Wd). Afterward, samples were immersed in culture media. Swollen samples were removed from the culture media and weight was measured (Wt). The process was repeated every 5 min for the first 30 min and then every 30 min until a swelling equilibrium was reached. Degree of swelling (swelling ratio), swelling rate, equilibrium swelling ratio, and percentage equilibrium liquid content were measured using the following equations [Ref: Alok's papers]:

$$Swelling ratio = \frac{{W}_{t} - {W}_{d}}{{W}_{d}} = degree of swelling \qquad ( 1 )$$

$$Swelling rate = weight change per unit time = \frac{{W}_{t + Δ t} - {W}_{t}}{Δ t} \qquad ( 2 )$$

$$Equilibrium swelling ratio = \frac{{W}_{equ} - {W}_{d}}{{W}_{d}} \qquad ( 3 )$$

$$Percentage equilibrium water content = \frac{{W}_{equ} - {W}_{d}}{{W}_{equ}} \times 100 \qquad ( 4 )$$

Where, Wₜ, W_{d}, W_{t+Δt}, and W_{equ} are the weight of swollen samples at time t, the weight of dried samples, weight of the swollen samples at time interval Δt, and weight of the swollen samples at equilibrium state, respectively.

### Osteogenic Differentiation of MSCs Grown Inside the 3D Scaffolds

For the osteogenic differentiation, alginate-collagen bioink was loaded with MSC cells (D18P15) and casted in a cylindrical PVA mold to make an scaffold of 9 mm diameter and 6 mm height. 400 μl bioink was added per scaffold with total 5.8×10⁵ cells/sample (cell density: 2.9×10⁶ cells/ml). After casting, scaffolds were stored at −80° C. for at least 24 hours, followed by thawing in a culture media (pre warmed to 37° C.) containing 0.05 M CaCl₂) for 10 min. Next, crosslinking media was replaced with fresh culture media and incubated for 30 minutes. The media was changed again after 2 hours and the after 24 hours. On the third day, the culture media of 12 scaffolds were changed with osteogenic differentiation media, while the rest remained in the culture media to be served as control. Media was changed twice a week throughout the study and samples were kept at 37° C. in a 5% CO₂ incubator at 95% humidity. During the study, scaffolds were kept for 73 days in culture media and for 70 days in osteogenic media. Upon completion of the study, scaffolds were gently washed twice with 1×PBS (without Ca and Mg), and half of the scaffolds were directly stored at −80° C. (with and without fixation with 10% formaldehyde at 4° C. for 16 h) for the H&E staining, von Kossa staining, and fluorescence microscopy for the study of osteoblasts markers. Fixed scaffolds were washed twice and stored in 1×PBS containing Ca and Mg at 6° C. until use for micro-CT and Cryo-SEM. In order to exclude the effect of scaffold material in the deposition on bone-like mineral on the scaffold surface, scaffolds without cells were incubated in similar conditions in culture media and osteogenic differentiation media.

### Micro-CT (Micro Computed Tomography)

To investigate the osteogenic activity by determining bone-like mineral deposition, scaffolds fixed in 10% formaldehyde were analyzed using micro-CT. For the imaging, scaffold was kept in a polystyrene holder, submersed in 1×PBS (with Ca, Mg). Data was collected at room temperature at 55 kV and 188 μA using 0.5 mm aluminum filter and 1° step size.

### Cryo-SEM (Cryo-Scanning Electron Microscope)

To study the cell-loaded scaffolds, after osteogenic differentiation, scaffolds fixed in 10% formaldehyde were sliced and adhered to an aluminum stub using cryoembedding solution. As mentioned in section 5.3, these thin sections were frozen in slush nitrogen and after sublimation of desired area and gold coating, samples were observed under a cryo-SEM, operated at an accelerating voltage of 5 kV and a working distance of 6 mm.

### H&E and Von Kossa Staining

To support the Cryo-SEM and micro-CT data, scaffolds (without fixation) were cryo-embedded and 14 mm thick cryosections were produced using cryo-microtome by slicing parallel to the vertical axis of cylindrical scaffolds. The sections were transferred on a glass slide and were stored at −80° C. until use.

For H&E and von Kossa staining, frozen sections were first baked at 40° C. for 20 min, followed by cooling to room temperature for 5 min. Next, these slides were transferred in a coupling jar and sectioned were fixed in 10% formaldehyde for 30 min. After fixation, slides were washed with 1×PBS for 2 min, and then with DI water for 2 min and 1 min.

For H&E staining, after baking and fixation, followed by washing, sections were stained in hematoxylin (cat. No. GHS216, Sigma Aldrich, USA) for 3 min then washed with 3 changes of tap water each 1 min (until blue color stops coming off slides) and then wash once with DI water. Slides were counterstained with Eosin (cat. No. HT110232, Sigma Aldrich, USA) for 2 min and then rinse by dipping 5 times in tap water. After removing the excess water by tapping slides on tissue paper, slides were air dried room temperature and then coverslip using permanent mount medium before imaging using light microscope.

For von Kossa staining, sections were kept in 5% silver nitrate solution (prepared in DI water) (cat. No. of silver nitrate) in a clear glass jar at room temperature and placed under ultraviolet light for 3 h under UV light in a biosafety cabinet. Next, slides were washed with 2 changes of DI water. To remove the un-reacted silver, slides were incubated in 5% sodium thiosulfate (prepared in DI water) or 5 min at room temperature. Slides were rinse three times in DI water and counterstained with nuclear fast red for 5 min. Slides were rinse for 2 min in running tap water, followed by 2 changes of DI water. Slides were dehydrated quickly with absolute alcohol, followed by coverslip using permanent mounting medium before imaging using light microscope. Calcium salts looks black-brown with nuclei and cytoplasm visible red and pink, respectively.

### Supplementary Data:

Considering effect of swelling on the dimensional/structural stability, we noted a significantly high swelling rate (˜48 mg/min) in the first 5 minutes, and then it decreased to ˜3 mg/min in the next 5 min when a freeze-dried cylindrical-shaped scaffold (diameter ×length: 10 mm×5 mm) submerged in 1×PBS (with Ca, Mg) at 37° C. (FIG. 10). After 10 min, we did not notice any change in the swelling rate. The measured value of equilibrium swelling ratio and percentage equilibrium water content were 29.531±0.481 and 96.723±0.052, respectively.

1. A method of making a frozen, three-dimensional, cell-laden bioink scaffold by additive manufacturing methods or casting, comprising:
i) providing an aqueous solution comprising an effective amount of a first biocompatible polymer;
ii) adding an effective amount of a cryoprotectant to the aqueous solution;
iii) optionally adding an effective amount of an agent to the aqueous solution or subjecting the aqueous solution to a condition that promotes crosslinking of the first biocompatible polymer;
iv) adding cells to the aqueous solution to make a cell-laden bioink aqueous solution;
v) casting the cell-laden bioink aqueous solution in a three-dimensional mold at a subzero temperature, bioprinting the cell-laden bioink aqueous solution at a subzero temperature, or infusing the cell-laden bioink aqueous solution into a solid scaffold at a subzero temperature to produce a frozen, three-dimensional, cell-laden bioink scaffold.

2. The method of claim 1, further comprising adding to the aqueous solution an effective amount of a second biocompatible polymer, or combining the aqueous solution comprising an effective amount of a first biocompatible polymer with a second aqueous solution comprising an effective amount of a second biocompatible polymer.

3. The method of claim 1, wherein the first biocompatible polymer comprises an alginate.

4. The method of claim 2, wherein the second biocompatible polymer comprises gelatin or collagen

5. (canceled)

6. The method of claim 2, wherein the weight ratio of the first biocompatible polymer to the second biocompatible polymer is from about 1:10 to about 10:1.

7. (canceled)

8. The method of claim 3, wherein alginate is present in the cell-laden bioink solution in an amount of from about 0.1% by weight to about 10% by weight.

9. (canceled)

10. The method of claim 4, wherein gelatin is present in the cell-laden bioink solution in an amount of about 0.1% by weight to about 10% by weight.

11. (canceled)

12. The method of claim 6, wherein collagen is present in the cell-laden bioink solution in an amount of about 0.15% by weight to about 10% by weight.

13. (canceled)

14. (canceled)

15. The method of claim 1, wherein the cryoprotectant is selected from DMSO, polyvinylpyrrolidone (PVP), sucrose, glycerol, polyethylene glycol (PEG), ethylene glycol (EG), Ficoll, polyvinyl alcohol, polyglycerol and combinations thereof.

16. (canceled)

17. (canceled)

18. The method of claim 1, wherein the agent that promotes crosslinking is a combination of N-(3-dimethylaminopropyl)-n′-ethylcarbodiimide hydrochloride (EDC) and N-hydroxysuccinimide (NHS), wherein EDC and NHS are added to the aqueous solution and the solution is incubated for a period of time to effect crosslinking.

19.-23. (canceled)

24. The method of claim 1, wherein the subzero temperature is maintained by placing the mold on dry ice or bioprinting on a substrate that is placed on dry ice.

25. (canceled)

26. (canceled)

27. The method of claim 1, wherein the cells comprise a heterogeneous or homogeneous population of cells and are selected from genetically engineered cells, differentiated cells, tissue specific stem cells, muscle stem cells, gut stem cells, intestinal stem cells, multipotent stem cells, embryonic stem cells, hematopoietic stem cells, cancer cells, progenitor cells, precursor cells, keratinocytes, melanocytes, neuronal cells, hepatic cells, epithelial cells, cardiomyocytes, cardiac progenitor cells, cardiac stem cells, muscle cells, fibroblasts, osteoblasts, endothelial cells, mesenchymal stem cells, induced pluripotent stem cells, and combinations thereof.

28. The method of claim 1, wherein the cell-laden bioink solution further comprises one or more bioactive molecules.

29. (canceled)

30. The method of claim 1, wherein the bioactive molecule is selected from a growth factor, cytokine, hormone, drug, immunosuppressant, antibiotic, biologic, antibody, chemotherapeutic agent, and combinations thereof.

31. The method of claim 1, wherein the cell-laden bioink solution further comprises one or more additional components, wherein the one or more additional components comprise polylactic acid (PLA), or extracellular matrix component selected from laminin, collagen, poly D (or L)-lysine, fibronectin, elastin, vitronectins, and combinations thereof.

32. (canceled)

33. (canceled)

34. The method of claim 1, wherein the frozen, cell-laden bioink scaffold is capable of being cryopreserved and stored at −80° C. for an extended period of time without significant damage to the scaffold or the cells within the scaffold.

35. The method of claim 1, further comprising thawing the frozen, three-dimensional, cell-laden bioink scaffold, wherein the frozen, three-dimensional, cell-laden bioink scaffold is optionally thawed in the presence of an effective amount of a crosslinker, wherein the three-dimensional, cell-laden bioink scaffold maintains structural integrity after thawing, wherein the three-dimensional, cell-laden bioink scaffold exhibits little or no interlayer mixing during casting or bioprinting and after thawing.

36.-40. (canceled)

41. The method of claim 1, wherein the thawed, three-dimensional, cell-laden bioink scaffold has a compressive strength of between about 2-3 kPa at 37° C. in submersion conditions, has a yield strength of about 1.5-2.0 kPa, and has a bulk elastic modulus of about 0.05-0.09 kPa, and is capable of supporting cellular proliferation, cellular differentiation, cellular migration, and/or tissue organization.

42.-44. (canceled)

45. A three-dimensional, cell-laden bioink scaffold produced according to the method of claim 1.

46. A method of treating a disease or condition in a subject, comprising engrafting the three-dimensional, cell-laden bioink scaffold according to claim 45 into the subject.
